(19) United States
(12) Patent Application Publication
(10) Pub. No.: US 2026/0193657 A1
(43) Pub. Date: Jul. 9, 2026

(54) COMPOSITIONS AND METHODS FOR CONTROLLING ARTHROPOD PARASITE AND PEST INFESTATION

(71) Applicant: GREENLIGHT BIOSCIENCES, INC., Lexington, MA (US)

(72) Inventors: Alex INBERG, Ballwin, MO (US); Mahak KAPOOR, Chesterfield, MO (US)

(21) Appl. No.: 19/426,137

(22) Filed: Dec. 19, 2025

Related U.S. Application Data
(62) Division of application No. 17/161,641, filed on Jan. 28, 2021.

(51) Int. Cl. C12N 15/113; A01K 51/00; A01N 57/16

(52) U.S. Cl. CPC C12N 15/113; A01K 51/00; A01N 57/16; C12N 2310/14

(57) ABSTRACT

This application provides and discloses anti-parasitic, anti-pest or insecticidal nucleic acid molecules and their calmodulin target genes for the control of arthropod parasites and pests. In particular, this application provides and discloses insecticidal nucleic acid molecules that target Varroa calmodulin gene sequences. This application further provides methods and compositions for the control and treatment of parasites and pests in Apis mellifera (honey bee) hives

### REFERENCE TO ELECTRONIC SEQUENCE LISTING

The application contains a Sequence Listing which has been submitted electronically in .XML format and is hereby incorporated by reference in its entirety. Said .XML copy, created on Dec. 18, 2025, is named “16206-012US3.xml” and is 134,404 bytes in size. The sequence listing contained in this .XML file is part of the specification and is hereby incorporated by reference herein in its entirety.

### FIELD OF THE DISCLOSURE

Methods and compositions for controlling parasite and pest infestations of arthropods are provided. Also provided are methods and compositions for controlling Varroa mite infestation in bees.

### BACKGROUND

Arthropods of various species are increasingly cultured on a commercial scale. Insects and their grubs are nutritious and are eaten both raw and cooked in many cultures. Crustaceans such as crabs, lobsters, crayfish, shrimp and prawns are farmed on a large commercial scale and are an important part of the human diet. In addition to the culture of arthropod species for food, arthropods are also cultured as part of pest management strategies, including for the biological control of other arthropods, for example the culture parasitic wasps for the control of roaches and fire ants. Arthropods may also serve as the source of raw materials such as dyes, drugs, medicines, and antibiotics. Growing with the increasing importance of arthropod culture, are various pests and parasites that destroy the arthropod colonies or greatly reduce the yields of products obtained from arthropod culture. Accordingly, there is an increasing need for methods to control arthropod pests and parasites.

Among the most important species of cultured arthropods is the honey bee. Honey bees, Apis mellifera, are required for the effective pollination of crops and are therefore critical to world agriculture. Honey bees also produce economically important products, including honey and bees wax. Honey bees are susceptible to a number of parasites and pathogens, including the ectoparasitic mite, Varroa destructor.

Varroa (Varroa destructor) mites are the number one parasite of managed honey bees (Apis mellifera) and the biggest global threat to commercial beekeeping (Rosenkranz et al. 2010). An adult mite typically enters the worker and drone brood cells before they are capped, primed by honeybee brood pheromone. The mite submerges into the brood food that the bees put inside the cell in anticipation of capping, most probably to avoid being recognized and removed by nurse bees. Following capping of the brood cells by the nurse bees, the mite adheres to the larva and starts to ingest bee larval hemolymph. This process primes oogenesis in the mites, and is followed several days later in laying of male and female eggs. Eventually, the adult Varroa exit the cell and cling onto the emerging bees. Varroa directly damages the honeybees in multiple ways, most notably by draining resources, adversely affecting the innate honey bee immune system, and by being a very effective vector of viruses (Di Prisco et al. 2011), some of which are known to replicate in the mite, thus dramatically increasing the viral load.

A safe, efficacious and long-lasting solution to the Varroa problem is an ongoing challenge that has yet to be met. Currently, beekeepers use a plethora of methods to control Varroa levels that include various chemical miticides, most of which have lost efficacy and are toxic and/or leave residues in wax and honey. Other methods include application of oxalic or formic acid, monoterpenes (thymol) and a variety of other management practices, with highly variable outcomes, including toxicity to the treated colonies. Breeding of bees for resistance to Varroa, such as selection for Hygienic behavior which results in the removal of infested brood, has provided a limited practical success.

Colony Collapse Disorder (CCD) of honeybees is threatening to annihilate U.S. and world agriculture. Indeed, in the recent outbreak of CCD in the U. S in the winter of 2006-2007, an estimated 25% or more of the 2.4 million honeybee hives were lost because of CCD. An estimated 23% of beekeeping operations in the United States suffered from CCD over the winter of 2006-2007, affecting an average of 45% of the beekeepers operations. In the winter of 2007-2008, the CCD action group of the USDA-ARS estimated that a total of 36% of all hives from commercial operations were destroyed by CCD.

CCD is characterized by the rapid loss from a colony of its adult bee population, with dead adult bees usually found at a distance from the colony. At the final stages of collapse, a queen is attended only by a few newly emerged adult bees. Collapsed colonies often have considerable capped brood and food reserves. The phenomenon of CCD was first reported in 2006; however, beekeepers noted unique colony declines consistent with CCD as early as 2004. Various factors such as mites and infectious agents, weather patterns, electromagnetic (cellular antennas) radiation, pesticides, poor nutrition and stress have been postulated as causes. To date, control of CCD has focused on Varroa mite control, sanitation and removal of affected hives, treating for opportunistic infections (such as Nosema) and improved nutrition. No effective preventative measures have been developed to date.

Varroa mites parasitize pupae and adult bees and reproduce in the pupal brood cells. The mites use their mouths to puncture the exoskeleton and feed on the bee's hemolymph. These wound sites in the exoskeleton harbor bacterial infections, such as Melissococcus pluton, which causes European foulbrood. In addition, to their parasitic effects, Varroa mites are suspected of acting as vectors for a number of honey bee pathogens, including deformed wing virus (DWV), Kashmir bee virus (KBV), acute bee paralysis virus (ABPV) and black queen cell virus (BQCV), and may weaken the immune systems of their hosts, leaving them vulnerable to infections. If left untreated Varroa infestations typically result in colony-level mortality.

Current methods of treating Varroa infestations are proving to be ineffective as the mites develop resistance to existing miticides. In addition, the use of such miticides may introduce injurious chemicals into honey that is intended for human consumption.

### SUMMARY OF THE INVENTION

The present disclosure provides for, and includes, selective insecticide compositions comprising an anti-parasitic, anti-pest or insecticidal nucleic acid molecule having a sequence that is essentially complementary or essentially identical to a region of a calmodulin gene sequence or an RNA transcribed therefrom. In some aspects, the composition further comprises an excipient.

Several embodiments relate to an anti-parasitic, anti-pest or insecticidal nucleic acid molecule that is essentially complementary or essentially identical to a region of a Varroa calmodulin gene sequence, or an RNA transcribed therefrom and does not comprise a sequence of 19 or more contiguous nucleotides that are 100% identical or complementary to a gene sequence of a non-target organism. In some embodiments, the anti-parasitic, anti-pest or insecticidal nucleic acid molecule comprises deletion of one or more nucleic acids in comparison to a reference Varroa calmodulin gene sequence selected from SEQ ID NOs: 1-4, 6, 23, 26-35, 69-89 and 93 such that the anti-parasitic, anti-pest or insecticidal nucleic acid molecule does not comprise a sequence of 19 or more contiguous nucleotides that are 100% identical or complementary to a gene sequence of a non-target organism. In some embodiments, the non-target organism gene sequence is an ortholog of the Varroa calmodulin gene sequence. In some embodiments, the non-target organism gene sequence is an homolog of the Varroa calmodulin gene sequence. In some embodiments, the non-target organism gene sequence is unrelated to the Varroa calmodulin gene sequence. In one aspect, the anti-parasitic, anti-pest or insecticidal nucleic acid molecule has at least one nucleotide deleted in said region in comparison to the targeted calmodulin gene sequence. In some embodiments, the anti-parasitic, anti-pest or insecticidal nucleic acid molecule has at least two, at least three, or at least four nucleotides deleted in said region in comparison to the targeted calmodulin gene sequence. In some embodiments, the at least two, at least three, or at least four deleted nucleotides are contiguous in the targeted calmodulin gene sequence. In other embodiments, the at least two, at least three, or at least four deleted nucleotides are not contiguous in the targeted calmodulin gene sequence. In some embodiments, the anti-parasitic, anti-pest or insecticidal nucleic acid comprises a sequence essentially identical or essentially complementary to a sequence selected from SEQ ID NOs: 90-92 and 94.

In one aspect, the nucleic acid molecule in the selective insecticide composition is a dsRNA. In some aspects, the dsRNA is an siRNA.

In one aspect, the anti-parasitic, anti-pest or insecticidal nucleic acid molecule has at least 80%, 85%, 90%, 95%, 96%, 97%, 98%, 99% or 100% sequence identity to a sequence selected from SEQ ID NOs: 1-4, 6, 23, 26-35, 69-89 and 93. In another aspect, the anti-parasitic, anti-pest or insecticidal nucleic acid molecule has at least 80%, 85%, 90%, 95%, 96%, 97%, 98%, 99%, or 100% sequence identity to a sequence selected from SEQ ID NOs: 90-92 and 94. In some aspects, the anti-parasitic, anti-pest or insecticidal nucleic acid molecule comprises at least 18 contiguous nucleotides of a sequence selected from SEQ ID NOs: 1-4, 6, 23, 26-35, and 69-93. In some aspects, the anti-parasitic, anti-pest or insecticidal nucleic acid does not comprise a sequence of 19 or more contiguous nucleotides that are 100% identical or complementary to a gene sequence of a non-target organism. In some aspects, the anti-parasitic, anti-pest or insecticidal nucleic acid molecule consists of a sequence selected from SEQ ID NOs: 1-4, 6, 23, 26-35, and 69-93.

In one aspect, the selective insecticide composition further comprises one or more anti-parasitic, anti-pest or insecticidal nucleic acid molecules that are essentially complementary or essentially identical to a first region of a calmodulin gene sequence. In some aspects, the one or more nucleic acid molecules comprise a second nucleic acid sequence complementary to a second region of a calmodulin gene sequence. In some embodiments, the anti-parasitic, anti-pest or insecticidal nucleic acid does not comprise a sequence of 19 or more contiguous nucleotides that are 100% identical or complementary to a gene sequence of a non-target organism. In some embodiments, the anti-parasitic, anti-pest or insecticidal nucleic acid molecule has at least one, at least two, at least three, or at least four nucleotide deletions in said first region and/or second region in comparison to a target calmodulin gene sequence. In some embodiments, the target calmodulin gene sequence is essentially identical or essentially complementary to a sequence selected from SEQ ID NOs: 1-4, 6, 23, 26-35, 69-89 and 93, or a fragment thereof. In some embodiments, the at least two, at least three, or at least four deleted nucleotides are contiguous in the target calmodulin gene sequence. In other embodiments, the at least two, at least three, or at least four deleted nucleotides are not contiguous in the target calmodulin gene sequence. In some embodiments, the anti-parasitic, anti-pest or insecticidal nucleic acid comprises a sequence essentially identical or essentially complementary to a sequence selected from SEQ ID NOs: 90-92.

In one aspect, the selective insecticide composition is bee-ingestible, bee-absorbable, mite-ingestible, or mite-absorbable.

In one aspect, the excipient is selected from the group consisting of protein, pollen, carbohydrate, polymer, liquid solvent, sugar syrup, sugar solid, and semi-solid feed. In some aspects, the liquid solvent is selected from the group consisting of sucrose solution and corn syrup solution. In some aspects, the protein is selected from the group consisting of pollen and soy protein. In another aspect, the excipient is a solid selected from sugar, a sugar substitute, or a sugar supplement. In some aspects, the sugar solid comprises sugar microparticles impregnated with dsRNA nucleic acid.

In one aspect, the instant application discloses bee-ingestible compositions comprising a bee feed and a nucleic acid molecule having a sequence that is essentially identical or essentially complementary to one or more regions of a calmodulin gene sequence, or an RNA transcribed therefrom. In some embodiments, the nucleic acid molecule is essentially complementary or essentially identical to a region of a Varroa calmodulin gene sequence, or an RNA transcribed therefrom, and does not comprise a sequence of 19 or more contiguous nucleotides that are 100% identical or complementary to a gene sequence of a non-target organism. In some embodiments, the nucleic acid molecule has at least one, at least two, at least three, or at least four nucleotides deleted in said one or more regions in comparison to a target Varroa calmodulin gene sequence. In some embodiments, the target Varroa calmodulin gene sequence is essentially complementary or essentially identical to a sequence selected from SEQ ID NOs: 1-4, 6, 23, 26-35, 69-89 and 93, or a fragment thereof. In some embodiments, the at least two, at least three, or at least four deleted nucleotides are contiguous in the targeted calmodulin gene sequence. In other embodiments, the at least two, at least three, or at least four deleted nucleotides are not contiguous in the targeted calmodulin gene sequence. In some embodiments, the nucleic acid molecule comprises a sequence essentially identical or essentially complementary to a sequence selected from SEQ ID NOs: 90-92 and 94. In some aspects, the bee feed comprises a bee food selected from the group consisting of corn syrup, a pollen substitute, pollen, a pollen patty, and a fondant. In some aspects, the bee feed further comprises one or more of a mineral salt, an essential oil, Brewers Yeast, yeast extract, trehalose, tryptone, dry milk, lecithin, and Vitamin C. Examples of essential oils include, but are not limited to, wintergreen oil, spearmint oil, peppermint oil, lemongrass oil and tea tree oil.

In another aspect, the instant application discloses a nucleic acid construct encoding an anti-parasitic, anti-pest or insecticidal nucleic acid sequence that is essentially identical or complementary to a region of a calmodulin gene sequence, or an RNA transcribed therefrom, operably linked to a promoter sequence functional in a host cell and capable of producing a dsRNA when introduced into said host cell. In some embodiments, the anti-parasitic, anti-pest or insecticidal nucleic acid is essentially complementary or essentially identical to a region of a targeted calmodulin gene sequence, or an RNA transcribed therefrom and does not comprise a sequence of 19 or more contiguous nucleotides that are 100% identical or complementary to a gene sequence of a non-target organism. In some embodiments, the anti-parasitic, anti-pest or insecticidal nucleic acid sequence comprises a deletion of at least one, at least two, at least three, or at least four nucleotides in comparison to a targeted calmodulin gene sequence. In some embodiments, the targeted calmodulin gene sequence is essentially identical or essentially complementary to a sequence selected from SEQ ID NOs: 1-4, 6, 23, 26-35, 69-89 and 93, or a fragment thereof. In some embodiments, the at least two, at least three, or at least four deleted nucleotides are contiguous in the targeted calmodulin gene sequence. In other embodiments, the at least two, at least three, or at least four deleted nucleotides are not contiguous in the targeted calmodulin gene sequence. In some embodiments, the anti-parasitic, anti-pest or insecticidal nucleic acid comprises a sequence essentially identical or essentially complementary to a sequence selected from SEQ ID NOs: 90-92 and 94. In some aspects, the nucleic acid construct further comprises at least one regulatory element selected from the group consisting of translation leader sequences, introns, enhancers, stem-loop structures, repressor binding sequences, termination sequences, pausing sequences, and polyadenylation recognition sequences. In some aspects, the host cell is a bacterial or yeast cell.

In another aspect, the instant application discloses a method of providing a composition to a honeybee, comprising providing the bee an effective amount of a composition comprising an anti-parasitic, anti-pest or insecticidal nucleic acid that is essentially identical or essentially complementary to one or more regions of a calmodulin gene sequence, or an RNA transcribed therefrom, whereby the nucleic acid is present in honeybee tissue. In some embodiments, the anti-parasitic, anti-pest or insecticidal nucleic acid is essentially complementary or essentially identical to a region of a targeted calmodulin gene sequence, or an RNA transcribed therefrom and does not comprise a sequence of 19 or more contiguous nucleotides that are 100% identical or complementary to a gene sequence of a non-target organism. In some embodiments, the non-target organism is a honeybee. In some embodiments, the anti-parasitic, anti-pest or insecticidal nucleic acid comprises deletion of at least one, at least two, at least three, or at least four nucleotides in said one or more regions in comparison to a targeted calmodulin gene sequence. In some embodiments, the targeted calmodulin gene sequence is essentially identical or essentially complementary to a sequence selected from SEQ ID NOs: 1-4, 6, 23, 26-35, 69-89 and 93, or a fragment thereof. In some embodiments, the at least two, at least three, or at least four deleted nucleotides are contiguous in the targeted calmodulin gene sequence. In other embodiments, the at least two, at least three, or at least four deleted nucleotides are not contiguous in the targeted calmodulin gene sequence. In some embodiments, the anti-parasitic, anti-pest or insecticidal nucleic acid comprises a sequence essentially identical or essentially complementary to a sequence selected from SEQ ID NOs: 90-92 and 94. In another aspect, the instant application discloses a method of treating or preventing disease in a honeybee colony, comprising providing an effective amount of a composition comprising an anti-parasitic, anti-pest or insecticidal nucleic acid that is essentially identical or essentially complementary to one or more regions of a calmodulin gene sequence to a honeybee whereby the nucleic acid is present in honeybee tissue. In some aspects, the calmodulin gene sequence is a Varroa destructor calmodulin gene sequence. In some embodiments, the anti-parasitic, anti-pest or insecticidal nucleic acid is essentially complementary or essentially identical to a region of a Varroa calmodulin gene sequence, or an RNA transcribed therefrom and does not comprise a sequence of 19 or more contiguous nucleotides that are 100% identical or complementary to a gene sequence of a non-target organism. In some embodiments, the anti-parasitic, anti-pest or insecticidal nucleic acid comprises deletion of at least one, at least two, at least three, or at least four nucleotides in one or more regions in comparison to a targeted calmodulin gene sequence. In some embodiments, the targeted calmodulin gene sequence is essentially complementary or essentially identical to a nucleic acid sequence selected from SEQ ID NOs: 1-4, 6, 23, 26-35, 69-89 and 93, or a fragment thereof. In some embodiments, the at least two, at least three, or at least four deleted nucleotides are contiguous in the targeted calmodulin gene sequence. In other embodiments, the at least two, at least three, or at least four deleted nucleotides are not contiguous in the targeted calmodulin gene sequence.

In another aspect, the instant application discloses a method of reducing parasitation of a bee by Varroa destructor, comprising providing the bee an effective amount of an anti-parasitic, anti-pest or insecticidal nucleic acid composition, wherein the nucleic acid is essentially identical or essentially complementary to one or more regions of a Varroa destructor calmodulin gene sequence, or an RNA transcribed therefrom, thereby reducing the parasitation of the bee by Varroa destructor. In some embodiments, the anti-parasitic, anti-pest or insecticidal nucleic acid comprises a sequence essentially identical or essentially complementary to a sequence selected from SEQ ID NOs: 90-92 and 94.

In another aspect, the instant application discloses a method of reducing the parasite load of a honeybee hive, comprising providing said hive an effective amount of an anti-parasitic, anti-pest or insecticidal nucleic acid that is essentially identical or essentially complementary to one or more regions of a parasite calmodulin gene sequence, or an RNA transcribed therefrom, whereby the parasite load of said hive is reduced. In some embodiments, the anti-parasitic, anti-pest or insecticidal nucleic acid does not comprise a sequence of 19 or more contiguous nucleotides that are 100% identical or complementary to a gene sequence of a non-target organism. In some embodiments, the anti-parasitic, anti-pest or insecticidal nucleic acid molecule comprises deletion of at least one, at least two, at least three, or at least four nucleotides in one or more regions in comparison to a targeted parasite calmodulin gene sequence. In some embodiments, the targeted parasite calmodulin gene sequence is essentially complementary or essentially identical to a nucleic acid sequence selected from SEQ ID NOs: 1-4, 6, 23, 26-35, 69-89 and 93. In some embodiments, the at least two, at least three, or at least four deleted nucleotides are contiguous in the targeted calmodulin gene sequence. In other embodiments, the at least two, at least three, or at least four deleted nucleotides are not contiguous in the targeted calmodulin gene sequence. In some embodiments, the anti-parasitic, anti-pest or insecticidal nucleic acid comprises a sequence essentially identical or essentially complementary to a sequence selected from SEQ ID NOs: 90-92 and 94.

In another aspect, the instant application discloses a method of selectively treating an arthropod species for parasites, comprising delivering an effective amount of an anti-parasitic, anti-pest or insecticidal nucleic acid that is essentially identical or essentially complementary to one or more regions of a parasite calmodulin gene sequence, or an RNA transcribed therefrom, to an arthropod species. In some embodiments, the anti-parasitic, anti-pest or insecticidal nucleic acid does not comprise a sequence of 19 or more contiguous nucleotides that are 100% identical or complementary to a gene sequence of a non-target organism. In some embodiments, the anti-parasitic, anti-pest or insecticidal nucleic acid has at least one, at least two, at least three, or at least four nucleotides deleted in one or more regions in comparison to a targeted arthropod calmodulin gene sequence. In some embodiments, the targeted arthropod calmodulin gene sequence is essentially complementary or essential identical to a sequence selected from SEQ ID NOs: 1-4, 6, 23, 26-35, 69-89 and 93, or a fragment thereof. In some embodiments, the at least two, at least three, or at least four deleted nucleotides are contiguous in the targeted calmodulin gene sequence. In other embodiments, the at least two, at least three, or at least four deleted nucleotides are not contiguous in the targeted calmodulin gene sequence. In some embodiments, the anti-parasitic, anti-pest or insecticidal nucleic acid comprises a sequence essentially identical or essentially complementary to a sequence selected from SEQ ID NOs: 90-92 and 94.

In another aspect, the instant application provides for, and discloses a method of treating or preventing Colony Collapse Disorder in a honeybee colony, comprising providing an effective amount of a composition to a honeybee colony comprising an anti-parasitic, anti-pest or insecticidal nucleic acid molecule having a sequence that is essentially identical to or essentially complementary to one or more regions of a Varroa destructor calmodulin gene sequence whereby the level of Varroa destructor infestation is reduced or prevented. In some embodiments, the anti-parasitic, anti-pest or insecticidal nucleic acid does not comprise a sequence of 19 or more contiguous nucleotides that are 100% identical or complementary to a gene sequence of a non-target organism. In some embodiments, the anti-parasitic, anti-pest or insecticidal nucleic acid has at least one, at least two, at least three, or at least four nucleotides deleted in one or more regions in comparison to a targeted calmodulin gene sequence. In some embodiments, the targeted arthropod calmodulin gene sequence is essentially complementary or essential identical to a sequence selected from SEQ ID NOs: 1-4, 6, 23, 26-35, 69-89 and 93, or a fragment thereof. In some embodiments, the at least two, at least three, or at least four deleted nucleotides are contiguous in the targeted calmodulin gene sequence. In other embodiments, the at least two, at least three, or at least four deleted nucleotides are not contiguous in the targeted calmodulin gene sequence.

### BRIEF DESCRIPTION OF THE DRAWINGS

FIG. 1 presents a phylogenetic tree for Calmodulin (CAM) genes from different species. The number immediately preceding the species name corresponds to a Sequence Identification Number (SEQ ID NO).

FIG. 2 presents the survival rate of mites exposed to a nucleic acid comprising a sequence identical or complementary to SEQ ID NO: 3 (CAM373) in a direct feeding bioassay at 3 day post treatment relative to a non treated control (CNTR) or a non-specific sequence (SCRAM, SEQ ID NO: 5).

FIG. 3 Panel A presents a gene expression analysis at five day post treatment with a nucleic acid comprising a sequence identical or complementary to SEQ ID NO: 3 (CAM373) or a nucleic acid comprising a sequence identical or complementary to SEQ ID NO: 4 (CAM186) relative to controls. Panel B shows the survival rate of mites exposed to nucleic acids comprising a sequence identical or complementary to SEQ ID NOS: 3 (CAM373) or 4 (CAM186) relative to controls.

FIG. 4 presents a mite load/100 bees of treated hives relative to untreated controls over a distinct time period.

FIG. 5 presents the % survival of mites treated with nucleic acids comprising a sequence identical or complementary to SEQ ID NO: 3, SEQ ID NO: 88 or SEQ ID NO: 89 relative to untreated (NTC) at Day 5 (D %) or Day 6 (D6) post-treatment.

FIG. 6 presents the % survival of mites treated with a nucleic acid comprising a sequence identical or complementary to SEQ ID NO: 3 or a mixture of nucleic acids comprising a sequence identical or complementary to SEQ ID NO: 88 or SEQ ID NO: 89 relative to untreated (NTC) at Day 5 (5), Day 6 (6) and Day 7 (7).

FIG. 7 presents the Varroa mite load/100 bees of treated hives relative to untreated controls over a 17 week time period. The leftmost bars represent hives treated with the non-specific sequence (SCRAM, SEQ ID NO: 5), the middle bars are hives left untreated, and the rightmost bar are hives treated with a nucleic acid comprising a sequence identical or complementary to SEQ ID NO: 3 (CAM 373).

FIG. 8 presents the % survival of the mites treated with a nucleic acid comprising a sequence identical or complementary to SEQ ID NO: 3, a nucleic acid comprising a sequence identical or complementary to SEQ ID NO: 90 or a nucleic acid comprising a sequence identical or complementary to SEQ ID NO: 92 relative to untreated (NTC) at Day 5 (D5) or Day 6 (D6).

FIG. 9 presents the % survival of the mites treated with a nucleic acid comprising a sequence identical or complementary to SEQ ID NO: 3, a nucleic acid comprising a sequence identical or complementary to SEQ ID NO: 91 or a mixture of nucleic acids comprising a sequence identical or complementary to SEQ ID NO: 90 or SEQ ID NO: 92 relative to untreated (NTC) at Day 4 (D4).

FIG. 10 presents the Calmodulin gene expression analysis as measured by QuantiGene™ (QG) of treatments for SEQ ID NO: 3, SEQ ID NO: 91 and a mixture of SEQ ID NO: 90 and SEQ ID NO: 92 relative to untreated (NTC) at Day 4.

FIG. 11 presents the Varroa mite load/100 bees of treated hives relative to untreated controls over a 19 week time period. The leftmost bars represent untreated hives, the middle bars are hives treated with a nucleic acid comprising a sequence identical or complementary to SEQ ID NO: 94 and the rightmost bar are hives treated with a nucleic acid comprising a sequence identical or complementary to SEQ ID NO: 91.

### DETAILED DESCRIPTION

Unless defined otherwise, technical and scientific terms as used herein have the same meaning as commonly understood by one of ordinary skill in the art. One skilled in the art will recognize many methods can be used in the practice of the present disclosure. Indeed, the present disclosure is in no way limited to the methods and materials described. Any references cited herein are incorporated by reference in their entireties. For purposes of the present disclosure, the following terms are defined below.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 1 is expressed in a DNA sequence format (e.g., reciting T for thymine), but it can refer to either a DNA sequence that corresponds to a mature Varroa destructor calmodulin nucleic acid sequence, or the RNA sequence of a mature Varroa destructor calmodulin molecule nucleic acid sequence. Similarly, though SEQ ID NO: 3 is expressed in a RNA sequence format (e.g., reciting U for uracil), depending on the actual type of molecule being described, SEQ ID NO: 3 can refer to either the sense or antisense strand of a dsRNA, or the sequence of a DNA molecule that corresponds to the RNA sequence shown. In any event, both DNA and RNA molecules having the sequences disclosed with any substitutes are envisioned.

As used herein the term “about” refers to +10%.

As used herein, the singular form “a”, “an” and “the” include plural references unless the context clearly dictates otherwise. For example, the term “a compound” or “at least one compound” may include a plurality of compounds, including mixtures thereof.

As used herein, “essentially identical” or “essentially complementary” refers to a nucleic acid (or at least one strand of a double-stranded nucleic acid or portion thereof, or a portion of a single strand nucleic acid) that hybridizes under physiological conditions to the endogenous gene, an RNA transcribed therefrom, or a fragment thereof, to effect regulation or suppression of the endogenous gene. For example, in some aspects, a nucleic acid has 100 percent sequence identity or at least about 83, 84, 85, 86, 87, 88, 89, 90, 91, 92, 93, 94, 95, 96, 97, 98, or 99 percent sequence identity when compared to a region of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more contiguous nucleotides in the target gene or RNA transcribed from the target gene. In some aspects, a nucleic acid has 100 percent sequence complementarity or at least about 83, 84, 85, 86, 87, 88, 89, 90, 91, 92, 93, 94, 95, 96, 97, 98, or 99 percent sequence complementarity when compared to a region of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more contiguous nucleotides in the target gene or RNA transcribed from the target gene. In some aspects, a nucleic acid has 100 percent sequence identity with or complementarity to one allele or one family member of a given target gene (coding or non-coding sequence of a gene). In some aspects, a nucleic acid has at least about 83, 84, 85, 86, 87, 88, 89, 90, 91, 92, 93, 94, 95, 96, 97, 98, or 99 percent sequence identity with or complementarity to multiple alleles or family members of a given target gene. In some aspects, a nucleic acid has 100 percent sequence identity with or complementarity to multiple alleles or family members of a given target gene.

In some aspects, the nucleic acid is essentially identical or essentially complementary to at least about 10, 15, 20, 25, 30, 35, 40, 45, 50, 55, 60, 65, 70 or more contiguous nucleotides of an endogenous calmodulin gene of a targeted pest, or an RNA transcribed therefrom. In some aspects, the nucleic acid does not comprise a sequence of 19 or more contiguous nucleotides that are 100% identical or complementary to a gene sequence of a non-target organism. In some aspects, the nucleic acid comprises at least one nucleotide deletion in comparison to a targeted calmodulin gene sequence selected from SEQ ID NOs: 1-4, 6, 23, 26-35, 69-89 and 93. In an aspect, the nucleic acid sequence has at least one nucleotide deleted compared an endogenous reference calmodulin gene sequence of a targeted pest, or an RNA transcribed therefrom such that the nucleic acid does not comprise a sequence of 19 or more contiguous nucleotides that are 100% identical or complementary to a gene sequence of a non-target organism. The nucleic acid may be a single-stranded DNA, a single-stranded RNA, a double-stranded RNA, a double-stranded DNA, or a double-stranded DNA/RNA hybrid. In some aspects, the targeted calmodulin gene sequence is a Varroa destructor calmodulin gene sequence. In an aspect, the targeted calmodulin gene sequence is a calmodulin gene sequence selected from SEQ ID NO: 1. In an aspect, the targeted calmodulin gene sequence is a calmodulin gene sequence selected from SEQ ID NO: 2. In an aspect, the targeted calmodulin gene sequence is a calmodulin gene sequence selected from SEQ ID NO: 3. In an aspect, the targeted calmodulin gene sequence is a calmodulin gene sequence selected from SEQ ID NO: 4. In an aspect, the targeted calmodulin gene sequence is a calmodulin gene sequence selected from SEQ ID NO: 69. In an aspect, the targeted calmodulin gene sequence is a calmodulin gene sequence selected from SEQ ID NO: 70. In an aspect, the targeted calmodulin gene sequence is a calmodulin gene sequence selected from SEQ ID NOs: 71-87. In an aspect, the targeted calmodulin gene sequence is a calmodulin gene sequence selected from SEQ ID NO: 88. In an aspect, the targeted calmodulin gene sequence is a calmodulin gene sequence selected from SEQ ID NO: 89. In an aspect, the targeted calmodulin gene sequence is a calmodulin gene sequence selected from SEQ ID NO: 93. In an aspect, the nucleic acid has at least one nucleotide deleted compared to an endogenous calmodulin gene sequence of a targeted pest, or an RNA transcribed therefrom. In an aspect, the nucleic acid has at least two, at least three, at least four, at five, at least six, at least seven, at least eight, at least nine, or at least ten nucleotides deleted compared to an endogenous calmodulin gene sequence of a targeted pest, or an RNA transcribed therefrom. In some aspects, the at least two, at least three, at least four, at five, at least six, at least seven, at least eight, at least nine, or at least ten deleted nucleotides are contiguous in targeted endogenous calmodulin gene sequences. In other aspects, the at least two, at least three, at least four, at five, at least six, at least seven, at least eight, at least nine, or at least ten deleted nucleotides are not contiguous in the targeted endogenous calmodulin gene sequences. In one aspect, the targeted calmodulin gene sequence is essentially complementary or essentially identical to a sequence selected from SEQ ID NOs: 1-4, 6, 23, 26-35, 69-89 and 93. In one aspect, the nucleic acid comprises a sequence selected from SEQ ID NOs: 90-92 and 94.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition. In an aspect according to the present disclosure, a composition may be used to treat an organism or colony of organisms for the effects of parasitation. In an aspect, a nucleic acid composition may be used to treat a host organism or colony for parasites. In an aspect, the host organism is a bee and the parasite is the mite, Varroa destructor.

As used herein, the phrase “RNA silencing” refers to a group of regulatory mechanisms (e.g. RNA interference (RNAi), transcriptional gene silencing (TGS), post-transcriptional gene silencing (PTGS), quelling, co-suppression, and translational repression) mediated by RNA molecules which result in the inhibition or “silencing” of the expression of a corresponding protein-coding gene or bee pathogen RNA sequence. RNA silencing has been observed in many types of organisms, including plants, animals, and fungi. In aspects according the present disclosure, nucleic acid compositions provide for RNA silencing. In certain aspects, the nucleic acid compositions provide for RNA silencing and mortality in a parasite.

As used herein, the term “RNA silencing agent” refers to a nucleic acid which is substantially homologous or complementary to a polynucleotide sequence of a target gene or an RNA expressed from the target gene or a fragment thereof and is capable of inhibiting or “silencing” the expression of a target gene. RNA silencing agents are generally described in relation to the target sequence against which the RNA silencing agent is directed. The target sequence can include, but is not limited to, a promoter region, 5′ untranslated regions, transcript encoding regions that can include intronic regions, 3′ untranslated regions, or combinations of these regions. In some embodiments, the target sequence can include coding or non-coding sequence or both. In some embodiments, the target sequence is identical to or complementary to a messenger RNA, e.g., in embodiments where the target sequence is a cDNA. In some embodiments, the target sequence is a native calmodulin gene sequence that is endogenously expressed in a target pest. In some embodiments, the target sequence is a calmodulin gene sequence selected from SEQ ID NOs: 1-4, 6, 23, 26-35, 69-89 and 93, or a part thereof. In some embodiments, the reference calmodulin gene sequence comprises at least 18 contiguous nucleotides, at least 19 contiguous nucleotides, at least 20 contiguous nucleotides, at least 21 contiguous nucleotides, at least 22 contiguous nucleotides or more of a sequence selected from SEQ ID NOs: 1-4, 6, 23, 26-35, 69-89 and 93.

In certain aspects, the RNA silencing agent is capable of preventing complete processing (e.g., the full translation and/or expression) of an mRNA molecule through a post-transcriptional silencing mechanism. RNA silencing agents can be single- or double-stranded RNA or single- or double-stranded DNA or double-stranded DNA/RNA hybrids or modified analogues thereof. In some aspects, the RNA silencing agents are selected from the group consisting of (a) a single-stranded RNA molecule (ssRNA), (b) a ssRNA molecule that self-hybridizes to form a double-stranded RNA molecule, (c) a double-stranded RNA molecule (dsRNA), (d) a single-stranded DNA molecule (ssDNA), (e) a ssDNA molecule that self-hybridizes to form a double-stranded DNA molecule, and (f) a single-stranded DNA molecule including a modified Pol III gene that is transcribed to an RNA molecule, (g) a double-stranded DNA molecule (dsDNA), (h) a double-stranded DNA molecule including a modified Pol III promoter that is transcribed to an RNA molecule, (i) a double-stranded, hybridized RNA/DNA molecule, or combinations thereof. In some aspects these polynucleotides include chemically modified nucleotides or non-canonical nucleotides. In some aspects, the RNA silencing agents are noncoding RNA molecules, for example RNA duplexes comprising paired strands, as well as precursor RNAs from which such small non-coding RNAs can be generated. In some aspects, the RNA silencing agents are dsRNAs such as siRNAs, miRNAs and shRNAs. In one aspect, the RNA silencing agent is capable of inducing RNA interference. In another aspect, the RNA silencing agent is capable of mediating translational repression. In an aspect, the RNA silencing agent is capable of inhibiting the expression of a calmodulin gene. In another aspect, the RNA silencing agent is capable of being used in methods to inhibit the expression of a target gene and thereby kill a target organism. In certain aspects, the target gene is a calmodulin gene and the target organism is Varroa destructor.

RNA interference refers to the process of sequence-specific post-transcriptional gene silencing in animals mediated by small RNAs. The corresponding process in plants is commonly referred to as post-transcriptional gene silencing or RNA silencing and is also referred to as quelling in fungi. While not being limited to any particular theory, the process of post-transcriptional gene silencing is thought to be an evolutionarily-conserved cellular defense mechanism used to prevent the expression of foreign genes and is commonly shared by diverse flora and phyla.

Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA. In aspects according to the present disclosure, a nucleic acid composition results in RNA interference in a target organism. In certain aspects, the nucleic acid composition results in RNA interference in Varroa destructor when present in the host organism, the bee. According to aspects of the present disclosure, a selective insecticide may cause RNA interference in the targeted organism, while having no RNA interference activity in non-target organisms.

As used herein, “small RNA” refers to any RNA molecule that is at least 15 base pairs in length, generally 15-30 nucleotides long, preferably 20-24 nucleotides long. In aspects according to the present disclosure, a “small RNA” is greater than 50 base pairs in length. In an aspect, the small RNA is greater than 50 base pairs in length but less than about 500 base pairs. In an aspect, the small RNA is greater than 100 base pairs in length but less than about 500 base pairs. In an aspect, the small RNA is greater than 200 base pairs in length but less than about 500 base pairs.

A small RNA can be either double-stranded or single-stranded. Small RNA includes, without limitation, miRNA (microRNA), ta-siRNA (trans activating siRNA), siRNA, activating RNA (RNAa), nat-siRNA (natural anti-sense siRNA), hc-siRNA (heterochromatic siRNA), cis-acting siRNA, ImiRNA (long miRNA), IsiRNA (long siRNA) and easiRNA (epigenetically activated siRNA) and their respective precursors. In some embodiments, siRNA molecules of the disclosure are miRNA molecules, ta-siRNA molecules and RNAa molecules and their respective precursors. A small RNA may be processed in vivo by an organism to an active form. According to aspects of the present disclosure, a selective insecticide may be a small RNA.

In aspects according to the present disclosure, a small RNA is provided directly in a composition. In other aspects, a small RNA is produced by in vivo by an organism from either a DNA or an RNA precursor. In some aspects, the small RNA is produced as a product of a transgene in an organism, for example a yeast or bacterial cell. In certain aspects, a small RNA produced as a product of a transgene is produced as a precursor that is processed in vivo after ingestion or absorption by an organism. In other aspects, a small RNA produced as a product of a transgene is produced as a precursor that is processed in vivo after ingestion or absorption by an organism.

In some aspects, the RNA silencing agent may be an artificial microRNA. As used herein, an “artificial microRNA” (amiRNA) is a type of miRNA which is derived by replacing native miRNA duplexes from a natural miRNA precursor. Generally, an artificial miRNA is a non-naturally-existing miRNA molecule produced from a pre-miRNA molecule scaffold engineered by exchanging a miRNA sequence of a naturally-existing pre-miRNA molecule for a sequence of interest which corresponds to the sequence of an artificial miRNA. In aspects according to the present disclosure a nucleic acid composition may be an amiRNA composition.

Various studies demonstrate that long dsRNAs can be used to silence gene expression without inducing the stress response or causing significant off-target effects—see for example (Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134). The present disclosure provides for, and includes, methods and compositions having long dsRNAs.

As used herein, with respect to a nucleic acid sequence, nucleic acid molecule, or a gene, the term “natural” or “native” means that the respective sequence or molecule is present in a wild-type organism, that has not been genetically modified or manipulated by man. A small RNA molecule naturally targeting a target gene means a small RNA molecule present in a wild-type organism, the cell has not been genetically modified or manipulated by man which is targeting a target gene naturally occurring in the respective organism.

As used herein, the terms “homology” and “identity” when used in relation to nucleic acids, describe the degree of similarity between two or more nucleotide sequences. The percentage of “sequence identity” between two sequences is determined by comparing two optimally aligned sequences over a comparison window, such that the portion of the sequence in the comparison window may comprise additions or deletions (gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. A sequence that is identical at every position in comparison to a reference sequence is said to be identical to the reference sequence and vice-versa. An alignment of two or more sequences may be performed using any suitable computer program. For example, a widely used and accepted computer program for performing sequence alignments is CLUSTALW v1.6 (Thompson, et al. Nucl. Acids Res., 22:4673-4680, 1994).

As used herein, the terms “exogenous polynucleotide” and “exogenous nucleic acid molecule” relative to an organisms refer to a heterologous nucleic acid sequence which is not naturally expressed within that organism. An exogenous nucleic acid molecule may be introduced into an organism in a stable or transient manner. An exogenous nucleic acid molecule may comprise a nucleic acid sequence which is identical or partially homologous to an endogenous nucleic acid sequence of the organism or a pest or pathogen of that organism. In certain aspects, an “exogenous polynucleotide” and “exogenous nucleic acid molecule” may refer to a parasite nucleic acid sequence expressed or present in a host, either transiently or stably. The present disclosure provides for, and includes, compositions comprising exogenous polynucleotides and exogenous nucleic acid molecules and methods for introducing them into a target organism. In some aspects, the present disclosure provides for, and includes, compositions comprising exogenous polynucleotides and exogenous nucleic acid molecules and methods for introducing them into a non-target organism that is a host to the target organism.

As used herein, a “control organism” means an organism that does not contain the recombinant DNA, small RNA, or other nucleic acid (e.g., protein, miRNA, small RNA-resistant target mRNA, dsRNA, target mimic) that provides for control of a pest or parasite. Control organisms are generally from same species and of the same developmental stage which is grown under the same growth conditions as the treated organism. Similarly, a “control colony” means a colony of organisms that do not contain the recombinant DNA, small RNA, or other nucleic acid (e.g., protein, miRNA, small RNA-resistant target mRNA, target mimic) that provides for control of a pest or parasite. Control colonies of organisms are generally from same species and of the same developmental stage which are grown under the same growth conditions as the treated colony of organisms. As a non-limiting example, a control organism could be a bee provided with a composition that does not contain a nucleic acid of the present disclosure. In another non-limiting example, a control organism could be a bee provided with a composition that contains a nucleic acid that does not act a an RNA silencer in either a bee or a parasite, such as SEQ ID NO: 5.

As used herein, the terms “improving,” “improved,” “increasing,” and “increased” refer to at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or greater increase in an organism or colony population, in increased productivity of an organism or colony (e.g., increased honey productions), increase growth rate of an organism or colony, or increased reproductive rate as compared to a control organism or colony. The present disclosure provides for methods of improving the health of an organism or colony by providing a selective insecticidal composition.

As used herein, “a reduction” of the level of an agent such as a protein or mRNA means that the level is reduced relative to an organism or colony lacking a nucleic acid capable of reducing the agent. Also as used herein, “a reduction” in reference to parasitation or parasite load, means that the level is reduced relative to an organism or colony lacking a nucleic acid, such as a dsRNA molecule, capable of reducing the viability, fecundity or number of the parasite. The present disclosure provides for, and includes, methods and compositions for reducing the level of a protein or mRNA and reducing the level or number of parasites.

As used herein, the term “at least a partial reduction” of the level of an agent, such as a protein or mRNA, means that the level is reduced at least 25% relative to an organism or colony lacking a nucleic acid, such as a dsRNA molecule, capable of reducing the agent. Also as used herein, “at least a partial reduction” in reference to parasitation or parasite load, means that the level is reduced at least 25% relative to an organism or colony lacking a nucleic acid, such as a dsRNA molecule, capable of reducing the viability, fecundity or number of the parasite. The present disclosure provides for, and includes, methods and compositions for at least partially reducing the level of a protein or mRNA and at least partially reducing the level or number of parasites.

As used herein, “a substantial reduction” of the level of an agent such as a protein or mRNA means that the level is reduced relative to an organism or colony lacking a nucleic acid, such as a dsRNA molecule, capable of reducing the agent, where the reduction of the level of the agent is at least 75%. Also as used herein, “a substantial reduction” in reference to parasitation or parasite load, means that the level is reduced at least 75% relative to an organism or colony lacking a nucleic acid, such as a dsRNA molecule, capable of reducing the viability, fecundity or number of the parasite. The present disclosure provides for, and includes, methods and compositions for substantially reducing the level of a protein or mRNA and substantially reducing the level or number of parasites.

As used herein, “an effective elimination” of an agent such as a protein or mRNA is relative to an organism or colony lacking a dsRNA molecule capable of reducing the agent, where the reduction of the level of the agent is greater than 95%. An agent, such as a dsRNA molecule, is preferably capable of providing at least a partial reduction, more preferably a substantial reduction, or most preferably effective elimination of another agent such as a protein or mRNA, or a parasite, wherein the agent leaves the level of a second agent, or host organism, essentially unaffected, substantially unaffected, or partially unaffected. Also as used herein, “an effective elimination” in reference to parasitation or parasite load, means that the level is reduced at least 95% relative to an organism or colony lacking a nucleic acid, such as a dsRNA molecule, capable of reducing the viability, fecundity or number of the parasite. The present disclosure provides for, and includes, methods and compositions for the effective elimination of a protein or mRNA and effectively eliminating parasites.

As used herein, the terms “suppress,” “repress,” and “downregulate” when referring to the expression or activity of a nucleic acid molecule in an organism are used equivalently herein and mean that the level of expression or activity of the nucleic acid molecule in a cell of an organism after applying a method of the present disclosure is lower than its expression or activity in the cell of an organism before applying the method, or compared to a control organism lacking a nucleic acid molecule of the disclosure. The present disclosure provides for, and includes, methods and compositions for suppressing, repressing and down-regulating the level of a protein or mRNA and suppressing, repressing and down-regulating the level or number of parasites.

The terms “suppressed,” “repressed” and “downregulated” as used herein are synonymous and mean herein lower, preferably significantly lower, expression or activity of a targeted nucleic acid molecule. Also as used herein, “suppressed,” “repressed” and “downregulated” in reference to parasitation or parasite load, means that the level of parasitation or parasite load is lower, preferably significantly lower, relative to an organism or colony lacking a nucleic acid, such as a dsRNA molecule, capable of reducing the viability, fecundity or number of the parasite. The present disclosure provides for, and includes, methods and compositions for suppressing, repressing and down-regulating the expression or activity of a protein or mRNA and suppressing, repressing and down-regulating the activity of parasites.

As used herein, a “suppression,” “repression,” or “downregulation” of the level or activity of an agent such as a protein, mRNA, or RNA means that the level or activity is reduced relative to a substantially identical cell, organism or colony grown under substantially identical conditions, lacking a nucleic acid molecule of the disclosure, for example, lacking the region complementary to a dsRNA or siRNA, the recombinant construct or recombinant vector of the disclosure. As used herein, “suppression,” “repression,” or “downregulation” of the level or activity of an agent, such as, for example, a preRNA, mRNA, rRNA, tRNA, snoRNA, snRNA expressed by the target gene, and/or of the protein product encoded by it, means that the amount is reduced by 10% or more, for example, 20% or more, preferably 30% or more, more preferably 50% or more, even more preferably 70% or more, most preferably 80% or more, for example, 90%, relative to a cell, organism or colony lacking a recombinant nucleic acid molecule of the disclosure. The present disclosure provides for, and includes, methods and compositions for suppression, repression and downregulation of an agent such as a protein, mRNA, RNA, or parasite compared to an untreated organism or colony.

As used herein, the term “arthropod” refers to both adult and pupa of invertebrate animals having an exoskeleton (external skeleton), a segmented body, and jointed appendages. Arthropods are members of the phylum Arthropoda and includes the insects, arachnids, and crustaceans. Arthropods according to the present disclosure, include but are not limited to Apis mellifera, Apis cerana, Trigona minima, Halictidae, Bombus sp., fleas, flies, lice, ticks, mites, and beneficial insects. The present disclosure provides for, and includes, methods and compositions for treating arthropods as either a host or as a parasite or pest.

In an aspect, an arthropod may be an insect. In certain aspects, an insect may be a bee. As used herein, the term “bee” refers to both an adult bee and pupal cells thereof. According to one aspect, the bee is in a hive. An adult bee is defined as any of several winged, hairy-bodied, usually stinging insects of the superfamily Apoidea in the order Hymenoptera, including both solitary and social species and characterized by sucking and chewing mouthparts for gathering nectar and pollen. Examples of bee species include, but are not limited to, Apis, Bombus, Trigona, Osmia and the like. In one aspect, bees include, but are not limited to bumblebees (Bombus terrestris), honeybees (Apis mellifera) (including foragers and hive bees) and Apis cerana. The present disclosure provides for, and includes, methods and compositions for treating bees as a host for parasites, such as Varroa mites.

According to one aspect, a bee is part of a colony. The term “colony” refers to a population of bees comprising dozens to typically several tens of thousands of bees that cooperate in nest building, food collection, and brood rearing. A colony normally has a single queen, the remainder of the bees being either “workers” (females) or “drones” (males). The social structure of the colony is maintained by the queen and workers and depends on an effective system of communication. Division of labor within the worker caste primarily depends on the age of the bee but varies with the needs of the colony. Reproduction and colony strength depend on the queen, the quantity of food stores, and the size of the worker force. Honeybees can also be subdivided into the categories of “hive bees”, usually for the first part of a workers lifetime, during which the “hive bee” performs tasks within the hive, and “forager bee”, during the latter part of the bee's lifetime, during which the “forager” locates and collects pollen and nectar from outside the hive, and brings the nectar or pollen into the hive for consumption and storage. The present disclosure provides for, and includes, methods and compositions for treating insect colonies.

As used herein, the term “pest” refers to both adult and immature forms of an organism that is invasive or prolific, detrimental, troublesome, noxious, destructive, a nuisance to either plants or animals, or ecosystems. A parasite is a type of pest. It is possible for an organism to be a pest in one setting but beneficial, domesticated, or acceptable in another.

As used herein, the term “parasite” refers to both adult and immature forms of organisms that directly benefit at the expense of another, host, organism, for example by feeding on the blood or fluids of the host, living intracellularly in a host organism cell, or living within a body of a host organism. Parasites include organisms that are animals, fungi, bacterial or plants and are identified by their negative or detrimental interaction with a host. In some aspects, a parasite as used herein may in turn serve as a host to a second parasite. In some aspects, a parasite and host may be of the same type of organism (e.g., an arthropod host and an arthropod parasite). Parasites include, but are not limited to, Acari (ticks, mites), Hippoboscoidea (flies), Ichneumonoidea (parasitic wasps), Oestridae (bot flies), Phthiraptera (lice), Siphonaptera (fleas), Tantulocarida, Pea crab, and Sacculina. As used herein, a pest may include both parasitic and non-parasitic life stages. The present disclosure provides for, and includes, methods and compositions for treating parasites. In an aspect, the parasite may be Varroa destructor. As provided for, and included, in the present disclosure, parasites and/or pests include Varroa destructor, Ixodes scapularis, Solenopsis invicta, Tetranychus urticae, Aedes aegypti, Culex quinquefasciatus, Acyrthosiphon pisum, and Pediculus humanus. In aspects according to the present disclosure, selective insecticides may be selective for Varroa destructor, Ixodes scapularis, Solenopsis invicta, Tetranychus urticae, Aedes aegypti, Culex quinquefasciatus, Acyrthosiphon pisum, and Pediculus humanus and inactive, or significantly less active, against a non-target organism, such as the host organism.

As used herein, the term “excipient” refers to any inactive substance in a formulation having an active ingredient such as an anti-parasitic, anti-pest or insecticidal nucleic acid, including without limitation dsRNA, small RNAs, miRNAs and antisense RNAs. In some embodiments, an excipient includes substances that may provide additional functionality to a composition that is distinct to the anti-parasitic, anti-pest, or insecticidal nucleic acids. Excipient functions include, but are not limited to “bulking agents,” “fillers,” “diluents,” and “carriers.” Bulking up allows convenient and accurate dispensation of compositions of the present disclosure. Excipients can also serve to facilitate ingestion of the compositions by organisms and include various carbohydrates, proteins, fatty acids, pollens, and pollen substitutes. Excipients can also serve to facilitate absorption of compositions by organisms an include, for example, both aqueous and non-aqueous solutions of active ingredients. Non-limiting examples of excipients include corn syrup, sugar syrup, sugar solid, sugar semi-solids, pollen, soy protein, pollen and protein mixtures. Excipients may further comprise attractants, buffers and nutrient supplements. Compositions of the present disclosure may be coated with, encapsulated in, dissolved in, mixed with, or otherwise combined with an excipient. As used herein, the term excipient may refer to a mixture of inactive substances.

This application provides and discloses anti-parasitic, anti-pest or insecticidal nucleic acid molecules that are substantially homologous or complementary to a polynucleotide sequence of a calmodulin target gene or an RNA expressed from the calmodulin target gene or a fragment thereof and functions to suppress the expression of the calmodulin target gene or produce a knock-down phenotype. In some aspects, the anti-parasitic, anti-pest or insecticidal nucleic acid does not comprise a sequence of 19 or more contiguous nucleotides that are 100% identical or complementary to a gene sequence of a non-target organism. In some aspects, the anti-parasitic, anti-pest or insecticidal nucleic acid molecule comprises deletion of one or more nucleic acids in comparison to a calmodulin target gene such that the anti-parasitic, anti-pest or insecticidal nucleic acid molecule does not comprise a sequence of 19 or more contiguous nucleotides that are 100% identical or complementary to a gene sequence of a non-target organism. The anti-parasitic, anti-pest or insecticidal nucleic acid molecules are capable of inhibiting or “silencing” the expression of a calmodulin target gene in a target species. In some embodiments, the anti-parasitic, anti-pest or insecticidal nucleic acid molecules do not affect expression of a calmodulin gene in a non-target organism, yet are capable of inhibiting or “silencing” the expression of a calmodulin target gene in a target species. The anti-parasitic, anti-pest or insecticidal nucleic acid molecules are generally described in relation to their “target sequence.” In some embodiments, the target sequence is selected from SEQ ID NOs. 1, 2 and 6-77. In some embodiments, the anti-parasitic, anti-pest or insecticidal nucleic acid molecules comprises at least one, at least two, at least three, or at least four nucleotides deleted in comparison to the target sequence. In some embodiments, the at least one, at least two, at least three, or at least four deleted nucleotides are contiguous in the target sequence. In other embodiments, the at least one, at least two, at least three, or at least four deleted nucleotides are not contiguous in the target sequence. The anti-parasitic, anti-pest or insecticidal nucleic acid molecules may be single-stranded DNA (ssDNA), single-stranded RNA (ssRNA), double-stranded RNA (dsRNA), double-stranded DNA (dsDNA), or double-stranded DNA/RNA hybrids. The nucleic acid molecules may comprise naturally-occurring nucleotides, modified nucleotides, nucleotide analogues or any combination thereof. In some embodiments, a anti-parasitic, anti-pest or insecticidal nucleic acid molecule may be incorporated within a larger polynucleotide, for example in a pri-miRNA molecule. In some embodiments, a anti-parasitic, anti-pest or insecticidal nucleic acid molecule may be processed into a small interfering RNA (siRNA). In some embodiments, nucleic acid molecules are provided or disclosed that are selectively anti-parasitical or miticidal, and methods of modulating expression or activity of their target genes to reduce or eliminate parasites from a colony or population. In some embodiments, nucleic acid molecules are provided or disclosed that are selectively anti-parasitical or miticidal, modulating expression or activity of the Varroa calmodulin gene to reduce or eliminate Varroa parasites from a colony or population of bees without affecting calmodulin gene in non-target organisms.

In aspects according to the present disclosure, a anti-parasitic, anti-pest or insecticidal nucleic acid molecule comprises a nucleotide sequence having at least 80%, 85%, 88%, 90%, 92%, 95%, 96%, 97%, 98%, 99% or 100% sequence identity to a sequence or a portion of a sequence selected from the group consisting of SEQ ID NOs: 1 to 94. In certain aspects, the nucleic acid molecule is selected from the group consisting of ssDNA, ssRNA, dsRNA, dsDNA, or DNA/RNA hybrids. Several embodiments relate to a dsRNA comprising a nucleotide sequence having at least 80%, 85%, 88%, 90%, 92%, 95%, 96%, 97%, 98%, or 99% sequence identity to a sequence or a portion of a sequence selected from the group consisting of SEQ ID NOs: 1 to 94. Several embodiments relate to a dsRNA comprising a nucleotide sequence having at least 100% sequence identity to a sequence or a portion of a sequence selected from the group consisting of SEQ ID NOs: 1 to 94. In another aspect, a DNA encoding at least one nucleic acid, such as a ssRNA or dsRNA, comprises a nucleotide sequence or a portion of a nucleotide sequence selected from the group consisting of SEQ ID NOs: 1 to 94, or having at least 80%, 85%, 88%, 90%, 92%, 95%, 96%, 97%, 98%, or 99% sequence identity to SEQ ID NOs: 1 to 94 or a portion thereof is provided. In yet another aspect, a recombinant DNA encoding at least one nucleic acid, such as a ssRNA or dsRNA, comprises a nucleotide sequence or a portion of a nucleotide sequence selected from the group consisting of SEQ ID NOs: 1 to 94, a heterologous promoter and a transcription terminator sequence are provided. In another aspect, the present disclosure provides a recombinant DNA encoding at least one nucleic acid, such as a ssRNA or dsRNA, that comprises a nucleotide sequence having at least 80%, 85%, 88%, 90%, 92%, 95%, 96%, 97%, 98%, 99% or 100% sequence identity to a sequence or a portion of a sequence selected from the group consisting of SEQ ID NOs: 1 to 94, and further comprising a heterologous promoter and a transcription terminator.

In aspects according to the present disclosure, a composition comprises an anti-parasitic, anti-pest or insecticidal nucleic acid molecule having 100 percent sequence identity to a region of 10 to 17 or more contiguous nucleotides in the target gene or RNA transcribed from the target gene. In an aspect, a composition comprises an anti-parasitic, anti-pest or insecticidal nucleic acid molecule having 100 percent sequence identity to a region of 18 to 25, or more contiguous nucleotides in the target gene or RNA transcribed from the target gene. In an aspect, a composition comprises an anti-parasitic, anti-pest or insecticidal nucleic acid molecule having 100 percent sequence identity to a region of 20 to 30, or more contiguous nucleotides in the target gene or RNA transcribed from the target gene. In an aspect, a composition comprises an anti-parasitic, anti-pest or insecticidal nucleic acid molecule having 100 percent sequence identity to a region of 25 to 35, or more contiguous nucleotides in the target gene or RNA transcribed from the target gene. In an aspect, a composition comprises an anti-parasitic, anti-pest or insecticidal nucleic acid molecule having 100 percent sequence identity to a region of 30 to 40, or more contiguous nucleotides in the target gene or RNA transcribed from the target gene. In an aspect, a composition comprises an anti-parasitic, anti-pest or insecticidal nucleic acid molecule having 100 percent sequence identity to a region of 40 to 50, or more contiguous nucleotides in the target gene or RNA transcribed from the target gene. In an aspect, a composition comprises an anti-parasitic, anti-pest or insecticidal nucleic acid molecule having 100 percent sequence identity to a region of 50 to 60, or more contiguous nucleotides in the target gene or RNA transcribed from the target gene. In an aspect, a composition comprises an anti-parasitic, anti-pest or insecticidal nucleic acid molecule having 100 percent sequence identity to a region of 45 to 60, or more contiguous nucleotides in the target gene or RNA transcribed from the target gene. In an aspect, a composition comprises an anti-parasitic, anti-pest or insecticidal nucleic acid molecule having 100 percent sequence identity to a region up to 60 contiguous nucleotides in the target gene or RNA transcribed from the target gene. In an aspect, a composition comprises an anti-parasitic, anti-pest or insecticidal nucleic acid molecule having 100 percent sequence identity to a region up to 50 contiguous nucleotides in the target gene or RNA transcribed from the target gene. In an aspect, a composition comprises an anti-parasitic, anti-pest or insecticidal nucleic acid molecule having 100 percent sequence identity to a region up to 40 contiguous nucleotides in the target gene or RNA transcribed from the target gene. In an aspect, a composition comprises an anti-parasitic, anti-pest or insecticidal nucleic acid molecule having 100 percent sequence identity to a region of at least 25 contiguous nucleotides in the target gene or RNA transcribed from the target gene. In an aspect, a composition comprises an anti-parasitic, anti-pest or insecticidal nucleic acid molecule having 100 percent sequence identity to a region of at least 35 contiguous nucleotides in the target gene or RNA transcribed from the target gene. In an aspect, a composition comprises an anti-parasitic, anti-pest or insecticidal nucleic acid molecule having 100 percent sequence identity to a region of at least 40 contiguous nucleotides in the target gene or RNA transcribed from the target gene. In an aspect, a composition comprises an anti-parasitic, anti-pest or insecticidal nucleic acid molecule having 100 percent sequence identity to a region of at least 50 contiguous nucleotides in the target gene or RNA transcribed from the target gene. In an aspect, a composition comprises an anti-parasitic, anti-pest or insecticidal nucleic acid molecule having 100 percent sequence identity to a region of at least 60 contiguous nucleotides in the target gene or RNA transcribed from the target gene. In an aspect, a target gene may be a gene comprising SEQ ID NO: 1 to 89 or 93. In an aspect, a target gene may be a gene comprising SEQ ID NO: 1. In an aspect, a target gene may be a gene comprising SEQ ID NO: 2. In an aspect, a target gene may be a gene comprising SEQ ID NO: 3. In an aspect, a target gene may be a gene comprising SEQ ID NO: 4. In an aspect, a target gene may be a gene comprising SEQ ID NO: 69. In an aspect, a target gene may be a gene comprising SEQ ID NO: 70. In an aspect, a target gene may be a gene comprising SEQ ID NO: 88. In an aspect, a target gene may be a gene comprising SEQ ID NO: 89. In an aspect, a target gene may be a gene comprising a sequence selected from SEQ ID NOs: 71-87. In an aspect, a target gene may be a gene comprising a sequence selected from SEQ ID NOs: 6-68.

In aspects according to the present disclosure, a composition comprises an anti-parasitic, anti-pest or insecticidal nucleic acid molecule having 99 percent sequence identity to a region of 10 to 17 or more contiguous nucleotides in the target gene or RNA transcribed from the target gene. In an aspect, a composition comprises an anti-parasitic, anti-pest or insecticidal nucleic acid molecule having 99 percent sequence identity to a region of 18 to 25, or more contiguous nucleotides in the target gene or RNA transcribed from the target gene. In an aspect, a composition comprises an anti-parasitic, anti-pest or insecticidal nucleic acid molecule having 99 percent sequence identity to a region of 20 to 30, or more contiguous nucleotides in the target gene or RNA transcribed from the target gene. In an aspect, a composition comprises an anti-parasitic, anti-pest or insecticidal nucleic acid molecule having 99 percent sequence identity to a region of 25 to 35, or more contiguous nucleotides in the target gene or RNA transcribed from the target gene. In an aspect, a composition comprises an anti-parasitic, anti-pest or insecticidal nucleic acid molecule having 99 percent sequence identity to a region of 30 to 40, or more contiguous nucleotides in the target gene or RNA transcribed from the target gene. In an aspect, a composition comprises an anti-parasitic, anti-pest or insecticidal nucleic acid molecule having 99 percent sequence identity to a region of 40 to 50, or more contiguous nucleotides in the target gene or RNA transcribed from the target gene. In an aspect, an anti-parasitic, anti-pest or insecticidal composition comprises a nucleic acid molecule having 99 percent sequence identity to a region of 50 to 60, or more contiguous nucleotides in the target gene or RNA transcribed from the target gene. In an aspect, a composition comprises an anti-parasitic, anti-pest or insecticidal nucleic acid molecule having 99 percent sequence identity to a region of 45 to 60, or more contiguous nucleotides in the target gene or RNA transcribed from the target gene. In an aspect, a composition comprises an anti-parasitic, anti-pest or insecticidal nucleic acid molecule having 99 percent sequence identity to a region up to 60 contiguous nucleotides in the target gene or RNA transcribed from the target gene. In an aspect, a composition comprises an anti-parasitic, anti-pest or insecticidal nucleic acid molecule having 99 percent sequence identity to a region up to 50 contiguous nucleotides in the target gene or RNA transcribed from the target gene. In an aspect, a composition comprises an anti-parasitic, anti-pest or insecticidal nucleic acid molecule having 99 percent sequence identity to a region up to 40 contiguous nucleotides in the target gene or RNA transcribed from the target gene. In an aspect, a composition comprises an anti-parasitic, anti-pest or insecticidal nucleic acid molecule having 99 percent sequence identity to a region of at least 25 contiguous nucleotides in the target gene or RNA transcribed from the target gene. In an aspect, a composition comprises an anti-parasitic, anti-pest or insecticidal nucleic acid molecule having 99 percent sequence identity to a region of at least 35 contiguous nucleotides in the target gene or RNA transcribed from the target gene. In an aspect, a composition comprises an anti-parasitic, anti-pest or insecticidal nucleic acid molecule having 99 percent sequence identity to a region of at least 40 contiguous nucleotides in the target gene or RNA transcribed from the target gene. In an aspect, a composition comprises an anti-parasitic, anti-pest or insecticidal nucleic acid molecule having 99 percent sequence identity to a region of at least 50 contiguous nucleotides in the target gene or RNA transcribed from the target gene. In an aspect, a composition comprises an anti-parasitic, anti-pest or insecticidal nucleic acid molecule having 99 percent sequence identity to a region of at least 60 contiguous nucleotides in the target gene or RNA transcribed from the target gene. In some aspects, an anti-parasitic, anti-pest or insecticidal nucleic acid has at least 98 percent sequence identity to a region of the target gene. In an aspect, an anti-parasitic, anti-pest or insecticidal nucleic acid has at least 97 percent sequence identity to a region of the target gene. In some aspects, an anti-parasitic, anti-pest or insecticidal nucleic acid has at least 96 percent sequence identity to a region of the target gene. In an aspect, an anti-parasitic, anti-pest or insecticidal nucleic acid has at least 95 percent sequence identity to a region of the target gene. In some aspects, an anti-parasitic, anti-pest or insecticidal nucleic acid has at least 94 percent sequence identity to a region of the target gene.

In an aspect, an anti-parasitic, anti-pest or insecticidal nucleic acid has at least 93 percent sequence identity to a region of the target gene. In some aspects, an anti-parasitic, anti-pest or insecticidal nucleic acid has at least 92 percent sequence identity to a region of the target gene. In an aspect, an anti-parasitic, anti-pest or insecticidal nucleic acid has at least 91 percent sequence identity to a region of the target gene. In an aspect, an anti-parasitic, anti-pest or insecticidal nucleic acid has at least about 83, 84, 85, 86, 87, 88, 89, 90 percent identity to a region of the target gene as provided above. In an aspect, a target gene may be a gene comprising SEQ ID NO: 1 to 89 or 93. In an aspect, a target gene may be a gene comprising SEQ ID NO: 1. In an aspect, a target gene may be a gene comprising SEQ ID NO: 2. In an aspect, a target gene may be a gene comprising SEQ ID NO: 3. In an aspect, a target gene may be a gene comprising SEQ ID NO: 4. In an aspect, a target gene may be a gene comprising SEQ ID NO: 69. In an aspect, a target gene may be a gene comprising SEQ ID NO: 70. In an aspect, a target gene may be a gene comprising SEQ ID NO: 88. In an aspect, a target gene may be a gene comprising SEQ ID NO: 89. In an aspect, a target gene may be a gene comprising sequence selected from SEQ ID NOs: 71-87. In an aspect, a target gene may be a gene comprising a sequence selected from SEQ ID NOs: 6-68.

In aspects according to the present disclosure, a composition comprises an anti-parasitic, anti-pest or insecticidal nucleic acid molecule having 100 percent sequence identity to a region of 10 to 17 or more contiguous nucleotides in to one allele or one family member of a given target gene (coding or non-coding sequence of a gene). In an aspect, a composition comprises an anti-parasitic, anti-pest or insecticidal nucleic acid molecule having 100 percent sequence identity to a region of 18 to 25, or more contiguous nucleotides to one allele or one family member of a given target gene). In an aspect, a composition comprises an anti-parasitic, anti-pest or insecticidal nucleic acid molecule having 100 percent sequence identity to a region of 20 to 30, or more contiguous nucleotides to one allele or one family member of a given target gene. In an aspect, a composition comprises an anti-parasitic, anti-pest or insecticidal nucleic acid molecule having 100 percent sequence identity to a region of 25 to 35, or more contiguous nucleotides to one allele or one family member of a given target gene. In an aspect, a composition comprises an anti-parasitic, anti-pest or insecticidal nucleic acid molecule having 100 percent sequence identity to a region of 30 to 40, or more contiguous nucleotides to one allele or one family member of a given target gene. In an aspect, a composition comprises an anti-parasitic, anti-pest or insecticidal nucleic acid molecule having 100 percent sequence identity to a region of 40 to 50, or more contiguous nucleotides to one allele or one family member of a given target gene. In an aspect, a composition comprises an anti-parasitic, anti-pest or insecticidal nucleic acid molecule having 100 percent sequence identity to a region of 50 to 60, or more contiguous nucleotides to one allele or one family member of a given target gene. In an aspect, a composition comprises an anti-parasitic, anti-pest or insecticidal nucleic acid molecule having 100 percent sequence identity to a region of 45 to 60, or more contiguous nucleotides to one allele or one family member of a given target gene. In an aspect, a composition comprises an anti-parasitic, anti-pest or insecticidal nucleic acid molecule having 100 percent sequence identity to a region up to 60 contiguous nucleotides to one allele or one family member of a given target gene. In an aspect, a composition comprises an anti-parasitic, anti-pest or insecticidal nucleic acid molecule having 100 percent sequence identity to a region up to 50 contiguous nucleotides to one allele or one family member of a given target gene. In an aspect, a composition comprises an anti-parasitic, anti-pest or insecticidal nucleic acid molecule having 100 percent sequence identity to a region up to 40 contiguous nucleotides to one allele or one family member of a given target gene. In an aspect, a composition comprises an anti-parasitic, anti-pest or insecticidal nucleic acid molecule having 100 percent sequence identity to a region of at least 25 contiguous nucleotides to one allele or one family member of a given target gene.

In an aspect, a composition comprises an anti-parasitic, anti-pest or insecticidal nucleic acid molecule having 100 percent sequence identity to a region of at least 35 contiguous nucleotides to one allele or one family member of a given target gene. In an aspect, a composition comprises an anti-parasitic, anti-pest or insecticidal nucleic acid molecule having 100 percent sequence identity to a region of at least 40 contiguous nucleotides to one allele or one family member of a given target gene. In an aspect, a composition comprises an anti-parasitic, anti-pest or insecticidal nucleic acid molecule having 100 percent sequence identity to a region of at least 50 contiguous nucleotides to one allele or one family member of a given target gene. In an aspect, a composition comprises an anti-parasitic, anti-pest or insecticidal nucleic acid molecule having 100 percent sequence identity to a region of at least 60 contiguous nucleotides to one allele or one family member of a given target gene. In an aspect, a target gene may be a gene comprising SEQ ID NO: 1 to 89. In an aspect, a target gene may be a gene comprising SEQ ID NO: 1. In an aspect, a target gene may be a gene comprising SEQ ID NO: 2. In an aspect, a target gene may be a gene comprising SEQ ID NO: 3. In an aspect, a target gene may be a gene comprising SEQ ID NO: 4. In an aspect, a target gene may be a gene comprising SEQ ID NO: 69. In an aspect, a target gene may be a gene comprising SEQ ID NO: 70. In an aspect, a target gene may be a gene comprising SEQ ID NO: 88. In an aspect, a target gene may be a gene comprising SEQ ID NO: 89. In an aspect, a target gene may be a gene comprising a sequence selected from SEQ ID NOs: 71-87. In an aspect, a target gene may be a gene comprising a sequence selected from SEQ ID NOs: 6-68. In an aspect, a target gene may be a gene comprising SEQ ID NO: 93.

In aspects according to the present disclosure, a composition comprises an anti-parasitic, anti-pest or insecticidal nucleic acid molecule having 99 percent sequence identity to a region of 10 to 17 or more contiguous nucleotides to one allele or one family member of a given target gene. In an aspect, a composition comprises an anti-parasitic, anti-pest or insecticidal nucleic acid molecule having 99 percent sequence identity to a region of 18 to 25, or more contiguous nucleotides to one allele or one family member of a given target gene. In an aspect, a composition comprises an anti-parasitic, anti-pest or insecticidal nucleic acid molecule having 99 percent sequence identity to a region of 20 to 30, or more contiguous nucleotides to one allele or one family member of a given target gene. In an aspect, a composition comprises an anti-parasitic, anti-pest or insecticidal nucleic acid molecule having 99 percent sequence identity to a region of 25 to 35, or more contiguous nucleotides to one allele or one family member of a given target gene. In an aspect, a composition comprises an anti-parasitic, anti-pest or insecticidal nucleic acid molecule having 99 percent sequence identity to a region of 30 to 40, or more contiguous nucleotides to one allele or one family member of a given target gene. In an aspect, a composition comprises an anti-parasitic, anti-pest or insecticidal nucleic acid molecule having 99 percent sequence identity to a region of 40 to 50, or more contiguous nucleotides to one allele or one family member of a given target gene. In an aspect, a composition comprises an anti-parasitic, anti-pest or insecticidal nucleic acid molecule having 99 percent sequence identity to a region of 50 to 60, or more contiguous nucleotides to one allele or one family member of a given target gene. In an aspect, a composition comprises an anti-parasitic, anti-pest or insecticidal nucleic acid molecule having 99 percent sequence identity to a region of 45 to 60, or more contiguous nucleotides to one allele or one family member of a given target gene. In an aspect, a composition comprises an anti-parasitic, anti-pest or insecticidal nucleic acid molecule having 99 percent sequence identity to a region up to 60 contiguous nucleotides to one allele or one family member of a given target gene. In an aspect, a composition comprises an anti-parasitic, anti-pest or insecticidal nucleic acid molecule having 99 percent sequence identity to a region up to 50 contiguous nucleotides to one allele or one family member of a given target gene. In an aspect, a composition comprises an anti-parasitic, anti-pest or insecticidal nucleic acid molecule having 99 percent sequence identity to a region up to 40 contiguous nucleotides to one allele or one family member of a given target gene. In an aspect, a composition comprises an anti-parasitic, anti-pest or insecticidal nucleic acid molecule having 99 percent sequence identity to a region of at least 25 contiguous nucleotides to one allele or one family member of a given target gene. In an aspect, a composition comprises an anti-parasitic, anti-pest or insecticidal nucleic acid molecule having 99 percent sequence identity to a region of at least 35 contiguous nucleotides to one allele or one family member of a given target gene. In an aspect, a composition comprises an anti-parasitic, anti-pest or insecticidal nucleic acid molecule having 99 percent sequence identity to a region of at least 40 contiguous nucleotides to one allele or one family member of a given target gene. In an aspect, a composition comprises an anti-parasitic, anti-pest or insecticidal nucleic acid molecule having 99 percent sequence identity to a region of at least 50 contiguous nucleotides to one allele or one family member of a given target gene. In an aspect, a composition comprises an anti-parasitic, anti-pest or insecticidal nucleic acid molecule having 99 percent sequence identity to a region of at least 60 contiguous nucleotides to one allele or one family member of a given target gene. In some aspects, an anti-parasitic, anti-pest or insecticidal nucleic acid has at least 98 percent sequence identity to a region of the target gene. In an aspect, an anti-parasitic, anti-pest or insecticidal nucleic acid has at least 97 percent sequence identity to a region of the target gene. In some aspects, an anti-parasitic, anti-pest or insecticidal nucleic acid has at least 96 percent sequence identity to a region of the target gene. In an aspect, an anti-parasitic, anti-pest or insecticidal nucleic acid has at least 95 percent sequence identity to a region of the target gene. In some aspects, an anti-parasitic, anti-pest or insecticidal nucleic acid has at least 94 percent sequence identity to a region of the target gene. In an aspect, an anti-parasitic, anti-pest or insecticidal nucleic acid has at least 93 percent sequence identity to a region of the target gene. In some aspects, an anti-parasitic, anti-pest or insecticidal nucleic acid has at least 92 percent sequence identity to a region of the target gene. In an aspect, an anti-parasitic, anti-pest or insecticidal nucleic acid has at least 91 percent sequence identity to a region of the target gene. In an aspect, an anti-parasitic, anti-pest or insecticidal nucleic acid has at least about 83, 84, 85, 86, 87, 88, 89, 90 percent identity to a region of the target gene as provided above. In an aspect, a target gene may be a gene comprising SEQ ID NO: 1 to 89. In an aspect, a target gene may be a gene comprising SEQ ID NO: 1. In an aspect, a target gene may be a gene comprising SEQ ID NO: 2. In an aspect, a target gene may be a gene comprising SEQ ID NO: 3. In an aspect, a target gene may be a gene comprising SEQ ID NO: 4. In an aspect, a target gene may be a gene comprising SEQ ID NO: 69. In an aspect, a target gene may be a gene comprising SEQ ID NO: 70. In an aspect, a target gene may be a gene comprising SEQ ID NO: 88. In an aspect, a target gene may be a gene comprising SEQ ID NO: 89. In an aspect, a target gene may be a gene comprising a sequence selected from SEQ ID NOs: 71-87. In an aspect, a target gene may be a gene comprising a sequence selected from SEQ ID NOs: 6-68. In an aspect, a target gene may be a gene comprising SEQ ID NO: 93.

In aspects according to the present disclosure, a composition comprises an anti-parasitic, anti-pest or insecticidal nucleic acid molecule having 100 percent sequence identity to a region of 10 to 17 or more contiguous nucleotides of identity with or complementarity to multiple alleles or family members of a given target gene. In an aspect, a composition comprises an anti-parasitic, anti-pest or insecticidal nucleic acid molecule having 100 percent sequence identity to a region of 18 to 25, or more contiguous nucleotides to one allele or one family member of a given target gene. In an aspect, a composition comprises an anti-parasitic, anti-pest or insecticidal nucleic acid molecule having 100 percent sequence identity to a region of 20 to 30, or more contiguous nucleotides of identity with or complementarity to multiple alleles or family members of a given target gene. In an aspect, a composition comprises an anti-parasitic, anti-pest or insecticidal nucleic acid molecule having 100 percent sequence identity to a region of 25 to 35, or more contiguous nucleotides of identity with or complementarity to multiple alleles or family members of a given target gene. In an aspect, a composition comprises an anti-parasitic, anti-pest or insecticidal nucleic acid molecule having 100 percent sequence identity to a region of 30 to 40, or more contiguous nucleotides of identity with or complementarity to multiple alleles or family members of a given target gene. In an aspect, a composition comprises an anti-parasitic, anti-pest or insecticidal nucleic acid molecule having 100 percent sequence identity to a region of 40 to 50, or more contiguous nucleotides of identity with or complementarity to multiple alleles or family members of a given target gene. In an aspect, a composition comprises an anti-parasitic, anti-pest or insecticidal nucleic acid molecule having 100 percent sequence identity to a region of 50 to 60, or more contiguous nucleotides of identity with or complementarity to multiple alleles or family members of a given target gene. In an aspect, a composition comprises an anti-parasitic, anti-pest or insecticidal nucleic acid molecule having 100 percent sequence identity to a region of 45 to 60, or more contiguous nucleotides of identity with or complementarity to multiple alleles or family members of a given target gene. In an aspect, a composition comprises an anti-parasitic, anti-pest or insecticidal nucleic acid molecule having 100 percent sequence identity to a region up to 60 contiguous nucleotides of identity with or complementarity to multiple alleles or family members of a given target gene. In an aspect, a composition comprises an anti-parasitic, anti-pest or insecticidal nucleic acid molecule having 100 percent sequence identity to a region up to 50 contiguous nucleotides of identity with or complementarity to multiple alleles or family members of a given target gene. In an aspect, a composition comprises an anti-parasitic, anti-pest or insecticidal nucleic acid molecule having 100 percent sequence identity to a region up to 40 contiguous nucleotides of identity with or complementarity to multiple alleles or family members of a given target gene. In an aspect, a composition comprises an anti-parasitic, anti-pest or insecticidal nucleic acid molecule having 100 percent sequence identity to a region of at least 25 contiguous nucleotides of identity with or complementarity to multiple alleles or family members of a given target gene. In an aspect, a composition comprises an anti-parasitic, anti-pest or insecticidal nucleic acid molecule having 100 percent sequence identity to a region of at least 35 contiguous nucleotides of identity with or complementarity to multiple alleles or family members of a given target gene. In an aspect, a composition comprises an anti-parasitic, anti-pest or insecticidal nucleic acid molecule having 100 percent sequence identity to a region of at least 40 contiguous nucleotides of identity with or complementarity to multiple alleles or family members of a given target gene. In an aspect, a composition comprises an anti-parasitic, anti-pest or insecticidal nucleic acid molecule having 100 percent sequence identity to a region of at least 50 contiguous nucleotides of identity with or complementarity to multiple alleles or family members of a given target gene. In an aspect, a composition comprises an anti-parasitic, anti-pest or insecticidal nucleic acid molecule having 100 percent sequence identity to a region of at least 60 contiguous nucleotides of identity with or complementarity to multiple alleles or family members of a given target gene. In an aspect, a target gene may be a gene comprising SEQ ID NO: 1 to 89. In an aspect, a target gene may be a gene comprising SEQ ID NO: 1. In an aspect, a target gene may be a gene comprising SEQ ID NO: 2. In an aspect, a target gene may be a gene comprising SEQ ID NO: 3. In an aspect, a target gene may be a gene comprising SEQ ID NO: 4. In an aspect, a target gene may be a gene comprising SEQ ID NO: 69. In an aspect, a target gene may be a gene comprising SEQ ID NO: 70. In an aspect, a target gene may be a gene comprising SEQ ID NO: 88. In an aspect, a target gene may be a gene comprising SEQ ID NO: 89. In an aspect, a target gene may be a gene comprising a sequence selected from SEQ ID NOs: 71-87. In an aspect, a target gene may be a gene comprising a sequence selected from SEQ ID NOs: 6-68. In an aspect, a target gene may be a gene comprising SEQ ID NO: 93.

In aspects according to the present disclosure, a composition comprises an anti-parasitic, anti-pest or insecticidal nucleic acid molecule having 99 percent sequence identity to a region of 10 to 17 or more contiguous nucleotides of identity with or complementarity to multiple alleles or family members of a given target gene. In an aspect, a composition comprises an anti-parasitic, anti-pest or insecticidal nucleic acid molecule having 99 percent sequence identity to a region of 18 to 25, or more contiguous nucleotides of identity with or complementarity to multiple alleles or family members of a given target gene. In an aspect, a composition comprises an anti-parasitic, anti-pest or insecticidal nucleic acid molecule having 99 percent sequence identity to a region of 20 to 30, or more contiguous nucleotides of identity with or complementarity to multiple alleles or family members of a given target gene. In an aspect, a composition comprises an anti-parasitic, anti-pest or insecticidal nucleic acid molecule having 99 percent sequence identity to a region of 25 to 35, or more contiguous nucleotides of identity with or complementarity to multiple alleles or family members of a given target gene. In an aspect, a composition comprises an anti-parasitic, anti-pest or insecticidal nucleic acid molecule having 99 percent sequence identity to a region of 30 to 40, or more contiguous nucleotides of identity with or complementarity to multiple alleles or family members of a given target gene. In an aspect, a composition comprises an anti-parasitic, anti-pest or insecticidal nucleic acid molecule having 99 percent sequence identity to a region of 40 to 50, or more contiguous nucleotides of identity with or complementarity to multiple alleles or family members of a given target gene. In an aspect, a composition comprises an anti-parasitic, anti-pest or insecticidal nucleic acid molecule having 99 percent sequence identity to a region of 50 to 60, or more contiguous nucleotides of identity with or complementarity to multiple alleles or family members of a given target gene. In an aspect, a composition comprises an anti-parasitic, anti-pest or insecticidal nucleic acid molecule having 99 percent sequence identity to a region of 45 to 60, or more contiguous nucleotides of identity with or complementarity to multiple alleles or family members of a given target gene. In an aspect, a composition comprises an anti-parasitic, anti-pest or insecticidal nucleic acid molecule having 99 percent sequence identity to a region up to 60 contiguous nucleotides of identity with or complementarity to multiple alleles or family members of a given target gene. In an aspect, a composition comprises an anti-parasitic, anti-pest or insecticidal nucleic acid molecule having 99 percent sequence identity to a region up to 50 contiguous nucleotides of identity with or complementarity to multiple alleles or family members of a given target gene. In an aspect, a composition comprises an anti-parasitic, anti-pest or insecticidal nucleic acid molecule having 99 percent sequence identity to a region up to 40 contiguous nucleotides of identity with or complementarity to multiple alleles or family members of a given target gene. In an aspect, a composition comprises an anti-parasitic, anti-pest or insecticidal nucleic acid molecule having 99 percent sequence identity to a region of at least 25 contiguous nucleotides of identity with or complementarity to multiple alleles or family members of a given target gene. In an aspect, a composition comprises an anti-parasitic, anti-pest or insecticidal nucleic acid molecule having 99 percent sequence identity to a region of at least 35 contiguous nucleotides of identity with or complementarity to multiple alleles or family members of a given target gene. In an aspect, a composition comprises an anti-parasitic, anti-pest or insecticidal nucleic acid molecule having 99 percent sequence identity to a region of at least 40 contiguous nucleotides of identity with or complementarity to multiple alleles or family members of a given target gene. In an aspect, a composition comprises an anti-parasitic, anti-pest or insecticidal nucleic acid molecule having 99 percent sequence identity to a region of at least 50 contiguous nucleotides of identity with or complementarity to multiple alleles or family members of a given target gene. In an aspect, a composition comprises an anti-parasitic, anti-pest or insecticidal nucleic acid molecule having 99 percent sequence identity to a region of at least 60 contiguous nucleotides of identity with or complementarity to multiple alleles or family members of a given target gene. In some aspects, an anti-parasitic, anti-pest or insecticidal nucleic acid has at least 98 percent sequence identity to a region of a target gene. In an aspect, an anti-parasitic, anti-pest or insecticidal nucleic acid has at least 97 percent sequence identity to a region of a target gene. In some aspects, an anti-parasitic, anti-pest or insecticidal nucleic acid has at least 96 percent sequence identity to a region of a target gene. In an aspect, an anti-parasitic, anti-pest or insecticidal nucleic acid has at least 95 percent sequence identity to a region of a target gene. In some aspects, an anti-parasitic, anti-pest or insecticidal nucleic acid has at least 94 percent sequence identity to a region of a target gene. In an aspect, an anti-parasitic, anti-pest or insecticidal nucleic acid has at least 93 percent sequence identity to a region of a target gene. In some aspects, an anti-parasitic, anti-pest or insecticidal nucleic acid has at least 92 percent sequence identity to a region of a target gene. In an aspect, an anti-parasitic, anti-pest or insecticidal nucleic acid has at least 91 percent sequence identity to a region of a target gene. In an aspect, an anti-parasitic, anti-pest or insecticidal nucleic acid has at least about 83, 84, 85, 86, 87, 88, 89, 90 percent identity to a region of a target gene as provided above. In an aspect, a target gene may be a gene comprising SEQ ID NO: 1 to 89. In an aspect, a target gene may be a gene comprising SEQ ID NO: 1. In an aspect, a target gene may be a gene comprising SEQ ID NO: 2. In an aspect, a target gene may be a gene comprising SEQ ID NO: 3. In an aspect, a target gene may be a gene comprising SEQ ID NO: 4. In an aspect, a target gene may be a gene comprising SEQ ID NO: 69. In an aspect, a target gene may be a gene comprising SEQ ID NO: 70. In an aspect, a target gene may be a gene comprising SEQ ID NO: 88. In an aspect, a target gene may be a gene comprising SEQ ID NO: 89. In an aspect, a target gene may be a gene comprising a sequence selected from SEQ ID NOs: 71-87. In an aspect, a target gene may be a gene comprising a sequence selected from SEQ ID NOs: 6-68. In an aspect, a target gene may be a gene comprising SEQ ID NO: 93.

In aspects according to the present disclosure, a composition comprises an anti-parasitic, anti-pest or insecticidal nucleic acid molecule having 100 percent sequence identity to a region of 10 to 17 or more contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 100 percent sequence identity to a region of 18 to 25, or more contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 100 percent sequence identity to a region of 20 to 30, or more contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 100 percent sequence identity to a region of 25 to 35, or more contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 100 percent sequence identity to a region of 30 to 40, or more contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 100 percent sequence identity to a region of 40 to 50, or more contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 100 percent sequence identity to a region of 50 to 60, or more contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 100 percent sequence identity to a region of 45 to 60, or more contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 100 percent sequence identity to a region up to 60 contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 100 percent sequence identity to a region up to 50 contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 100 percent sequence identity to a region up to 40 contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 100 percent sequence identity to a region of at least 25 contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 100 percent sequence identity to a region of at least 35 contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 100 percent sequence identity to a region of at least 40 contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 100 percent sequence identity to a region of at least 50 contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 100 percent sequence identity to a region of at least 60 contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a target gene may be a gene comprising SEQ ID NO: 1 to 89 or 93. In an aspect, a target gene may be a gene comprising SEQ ID NO: 1. In an aspect, a target gene may be a gene comprising SEQ ID NO: 2. In an aspect, a target gene may be a gene comprising SEQ ID NO: 3. In an aspect, a target gene may be a gene comprising SEQ ID NO: 4. In an aspect, a target gene may be a gene comprising SEQ ID NO: 69. In an aspect, a target gene may be a gene comprising SEQ ID NO: 70. In an aspect, a target gene may be a gene comprising SEQ ID NO: 88. In an aspect, a target gene may be a gene comprising SEQ ID NO: 89. In an aspect, a target gene may be a gene comprising a sequence selected from SEQ ID NOs: 71-87. In an aspect, a target gene may be a gene comprising a sequence selected from SEQ ID NOs: 6-68.

In aspects according to the present disclosure, a composition comprises an anti-parasitic, anti-pest or insecticidal nucleic acid molecule having 99 percent sequence identity to a region of 10 to 17 or more contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 99 percent sequence identity to a region of 18 to 25, or more contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 99 percent sequence identity to a region of 20 to 30, or more contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 99 percent sequence identity to a region of 25 to 35, or more contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 99 percent sequence identity to a region of 30 to 40, or more contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 99 percent sequence identity to a region of 40 to 50, or more contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, an anti-parasitic, anti-pest or insecticidal composition comprises a nucleic acid molecule having 99 percent sequence identity to a region of 50 to 60, or more contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 99 percent sequence identity to a region of 45 to 60, or more contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 99 percent sequence identity to a region up to 60 contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 99 percent sequence identity to a region up to 50 contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 99 percent sequence identity to a region up to 40 contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 99 percent sequence identity to a region of at least 25 contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 99 percent sequence identity to a region of at least 35 contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 99 percent sequence identity to a region of at least 40 contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 99 percent sequence identity to a region of at least 50 contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a composition comprises an anti-parasitic, anti-pest or insecticidal nucleic acid molecule having 99 percent sequence identity to a region of at least 60 contiguous nucleotides in the target gene or RNA transcribed from the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In some aspects, an anti-parasitic, anti-pest or insecticidal nucleic acid has at least 98 percent sequence identity to a region of the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, an anti-parasitic, anti-pest or insecticidal nucleic acid has at least 97 percent sequence identity to a region of the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In some aspects, an anti-parasitic, anti-pest or insecticidal nucleic acid has at least 96 percent sequence identity to a region of the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, an anti-parasitic, anti-pest or insecticidal nucleic acid has at least 95 percent sequence identity to a region of the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In some aspects, an anti-parasitic, anti-pest or insecticidal nucleic acid has at least 94 percent sequence identity to a region of the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, an anti-parasitic, anti-pest or insecticidal nucleic acid has at least 93 percent sequence identity to a region of the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In some aspects, an anti-parasitic, anti-pest or insecticidal nucleic acid has at least 92 percent sequence identity to a region of the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, an anti-parasitic, anti-pest or insecticidal nucleic acid has at least 91 percent sequence identity to a region of the target gene, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, an anti-parasitic, anti-pest or insecticidal nucleic acid has at least about 83, 84, 85, 86, 87, 88, 89, 90 percent identity to a region of the target gene as provided above, wherein the anti-parasitic, anti-pest or insecticidal nucleic acid molecule does not comprise a sequence that is 100% identical or complementary to 5, 6, 7, 8, 9, 10, 11, 12, 13, 14, 15, 16, 17, 18, 19, 20, 21, 22, 23, 24, 25 or more contiguous nucleotides of gene sequence of a non-target organism. In an aspect, a target gene may be a gene comprising SEQ ID NO: 1 to 89 or 93. In an aspect, a target gene may be a gene comprising SEQ ID NO: 1. In an aspect, a target gene may be a gene comprising SEQ ID NO: 2. In an aspect, a target gene may be a gene comprising SEQ ID NO: 3. In an aspect, a target gene may be a gene comprising SEQ ID NO: 4. In an aspect, a target gene may be a gene comprising SEQ ID NO: 69. In an aspect, a target gene may be a gene comprising SEQ ID NO: 70. In an aspect, a target gene may be a gene comprising SEQ ID NO: 88. In an aspect, a target gene may be a gene comprising SEQ ID NO: 89. In an aspect, a target gene may be a gene comprising sequence selected from SEQ ID NOs: 71-87.

In some embodiments, an anti-parasitic, anti-pest or insecticidal nucleic acid has at least one nucleotide deleted in comparison to a target gene sequence. In one aspect, the anti-parasitic, anti-pest or insecticidal nucleic acid does not contain a sequence of 5, 6, 7, 8, 9, 10, 11, 12, 13, 14, 15, 16, 17, 18, 19, 20, 21, 22, 23, 24, 25 or more consecutive nucleotides that is identical or complementary to a gene sequence in a non-targeting organism. In one embodiment, the non-targeting organism is a bee. In another embodiment, the non-targeting organism is a human. In another embodiment, the non-targeting organism is a monarch butterfly. In some embodiments, the anti-parasitic, anti-pest or insecticidal nucleic acid has at least one, at least two, at least three, at least four, at least five, at least six, at least seven, at least eight, at least nine, at least ten, at least eleven, at least twelve, at least thirteen, at least fourteen, at least fifteen, or more nucleotides deleted in comparison to a target sequence. In some embodiments, the at least two, at least three, at least four, at least five, or at least six nucleotides deleted are contiguous in the reference calmodulin gene sequence. In other embodiments, the at least one, at least two, at least three, at least four, at least five, at least six, at least seven, at least eight, at least nine, at least ten, at least eleven, at least twelve, at least thirteen, at least fourteen, at least fifteen, or more deleted nucleotides are not contiguous in the target sequence. In an aspect, the target sequence may be selected from SEQ ID NO: 1 to 89, 93 or a portion thereof. In an aspect, a target sequence may be a gene comprising SEQ ID NO: 1 or a portion thereof. In an aspect, a target sequence may be a gene comprising SEQ ID NO: 2 or a portion thereof. In an aspect, a target sequence may be a gene comprising SEQ ID NO: 3 or a portion thereof. In an aspect, a target sequence may be a gene comprising SEQ ID NO: 4 or a portion thereof. In an aspect, a target sequence may be a gene comprising SEQ ID NO: 69 or a portion thereof. In an aspect, a target sequence may be a gene comprising SEQ ID NO: 70 or a portion thereof. In an aspect, a target sequence may be a gene comprising SEQ ID NO: 88 or a portion thereof. In an aspect, a target sequence may be a gene comprising SEQ ID NO: 89 or a portion thereof. In an aspect, a target sequence may be a gene comprising a sequence selected from SEQ ID NOs: 71-87 or a portion thereof. In some embodiments, the anti-parasitic, anti-pest or insecticidal nucleic acid comprises a sequence that has at least about 80, 81, 82, 83, 84, 85, 86, 87, 88, 89, 90, 91, 92, 93, 94, 95, 96, 97, 98, 99 or 100 percent sequence identity to a sequence selected from SEQ ID NOs: 90-92 or 94, or a part thereof. In some embodiments, the anti-parasitic, anti-pest or insecticidal nucleic acid comprises the reverse complement of a sequence that has at least about 80, 81, 82, 83, 84, 85, 86, 87, 88, 89, 90, 91, 92, 93, 94, 95, 96, 97, 98, 99 or 100 percent sequence identity to a sequence selected from SEQ ID NOs: 90-92 or 94, or a part thereof. In some embodiments, the anti-parasitic, anti-pest or insecticidal nucleic acid comprises at least 18 contiguous nucleotides of a sequence selected from SEQ ID NOs: 90-92 or 94. In some embodiments, the anti-parasitic, anti-pest or insecticidal nucleic acid comprises a sequence selected from SEQ ID NOs: 90-92 or 94, or a part thereof. In some embodiments, the anti-parasitic, anti-pest or insecticidal nucleic acid comprises a sequence selected from SEQ ID NOs: 90-92 and 94, and the reverse complement of a sequence selected from SEQ ID NOs: 90-92 and 94.

This application provides and discloses compositions comprising an anti-parasitic, anti-pest or insecticidal nucleic acid molecule and an excipient substance. In an aspect, the excipient can be a combination of one or more inactive components. In some aspects, the excipient comprises a sugar. Examples of sugars include hexoses, disaccharides, trisaccharides and higher sugars. Excipient sugars include, for example, fructose, glucose, sucrose, trehalose, lactose, galactose, ribose. In other aspects the excipient comprises a sugar and a solvent. In other aspects, the excipient comprises a protein. In an aspect, the protein is a soy protein. In other aspects the excipient may be pollen. In aspects according to the present disclosure, the excipient may be a bee food. In some aspects, the excipient comprises Tryptone. In some aspects, the excipient comprises yeast extract. In some aspects, the excipient comprises an essential oil.

Bee feeding is common practice amongst bee-keepers, for providing both nutritional and other, for example, supplemental needs. Bees typically feed on honey and pollen, but have been known to ingest non-natural feeds as well. Bees can be fed various foodstuffs including, but not limited to Wheast (a dairy yeast grown on cottage cheese), soybean flour, yeast (e.g. brewer's yeast, torula yeast) and yeast products products-fed singly or in combination and soybean flour fed as a dry mix or moist cake inside the hive or as a dry mix in open feeders outside the hive. Also useful is sugar, or a sugar syrup. The addition of 10 to 12 percent pollen to a supplement fed to bees improves palatability. The addition of 25 to percent pollen improves the quality and quantity of essential nutrients that are required by bees for vital activity. Cane or beet sugar, isomerized corn syrup, and type-50 sugar syrup are satisfactory substitutes for honey in the natural diet of honey bees. The last two can be supplied only as a liquid to bees. Liquid feed can be supplied to bees inside the hive by, for example, any of the following methods: friction-top pail, combs within the brood chamber, division board feeder, boardman feeder, etc. Dry sugar may be fed by placing a pound or two on the inverted inner cover. A supply of water must be available to bees at all times. In one aspect, pan or trays in which floating supports-such as wood chips, cork, or plastic sponge—are present are envisaged. Detailed descriptions of supplemental feeds for bees can be found in, for example, USDA publication by Standifer, et al. 1977, entitled “Supplemental Feeding of Honey Bee Colonies” (USDA, Agriculture Information Bulletin No. 413).

In aspects according to the present disclosure, an anti-parasitic, anti-pest or insecticidal nucleic acid, for example a dsRNA, is absorbable. As used herein “absorbable,” refers to mechanisms the provide for the uptake of a nucleic acid that is not by ingestion. In an aspect, an anti-parasitic, anti-pest or insecticidal nucleic acid may be absorbed through the skin of an organism, or the exoskeleton of an arthropod. In an aspect, an absorbable nucleic acid is dissolved in an excipient. In other aspects, an absorbable nucleic acid is suspended in an excipient. Excipients for solvation or suspension may be aqueous or non-aqueous. In some aspects, the anti-parasitic, anti-pest or insecticidal nucleic acid is absorbed by a host organism and transferred to a parasitic organism by feeding. In other aspects, the anti-parasitic, anti-pest or insecticidal nucleic acid is absorbed by a host organism and transferred to a parasitic organism by absorption. In an aspect, an anti-parasitic, anti-pest or insecticidal nucleic acid of the present disclosure is absorbed directly by the parasite.

In aspects according to the present disclosure an anti-parasitic, anti-pest or insecticidal nucleic acid, for example a dsRNA, is combined with an excipient. In an aspect, the nucleic acid may be provided as a ratio of nucleic acid to excipient. In an aspect, the ratio may be one part nucleic acid to 4 parts excipient. In an aspect the ratio of nucleic acid to excipient may be 1:1, 1:2, 1:5, or 1:10. In other aspects, the ratio of nucleic acid to excipient may be 1:20, 1:25, 1:30, 1:40, or more. In an aspect, ratio of nucleic acid to excipient may be 1:50. In aspects according to the present disclosure, the ratio may be determined as a volume to volume (v/v) ratio, a weight:weight (w/w) ratio. In certain aspects, the ratio may be expressed as a weight:volume (w/v) ratio. In certain aspects, a nucleic acid and an excipient may be a dsRNA and an excipient.

In aspects according to the present disclosure, the composition may comprise a weight of an anti-parasitic, anti-pest or insecticidal nucleic acid combined with an excipient. In an aspect, the nucleic acid may comprise a percentage of the total weight of the composition. In an aspect, the nucleic acid may comprise about 0.1% by weight of the composition. In an aspect, the nucleic acid may comprise about 0.2% by weight of the composition. In an aspect, the nucleic acid may comprise about 0.3% by weight of the composition. In another aspect, the nucleic acid may comprise about 0.4% by weight of the composition. In an aspect, the nucleic acid may comprise up to 0.5% by weight of the composition. In an aspect, the nucleic acid may comprise up to 0.6% by weight of the composition. In an aspect, the nucleic acid may comprise up to 0.7% by weight of the composition. In an aspect, the nucleic acid may comprise up to 0.8% by weight of the composition. In another aspect, the nucleic acid may comprise up to 1.0% by weight of the composition. In other aspects, the nucleic acid may comprise up to 1.5% by weight of the composition. In yet other aspects, the nucleic acid may comprise up to 2.0% by weight, or 2.5% by weight of the composition. In certain aspects, a nucleic acid and an excipient may be a dsRNA and an excipient.

The present disclosure provides for, and includes, compositions having from 0.1% to 5% by weight of one or more anti-parasitic, anti-pest or insecticidal nucleic acids. In other aspects, a composition may comprise from 0.1 to 4%, 0.1 to 3%, 0.1 to 2%, 0.1 to 1%, 0.1 to 2%, 0.1 to 3%, or 0.1 to 4% by weight nucleic acid. In an aspect, a composition may comprise from 0.2% to 5% by weight nucleic acid. In other aspects, a composition may comprise from 0.2 to 4%, 0.2 to 3%, 0.2 to 2%, 0.2 to 1%, 0.2 to 2%, 0.2 to 3%, or 0.2 to 4% by weight nucleic acid. In other aspects, a composition may comprise up to 1%, up to 2%, up to 3%, up to 4%, or up to 5% nucleic acid. In other aspects, a composition may comprise up to 7.5%, up to 10%, or up to 15% nucleic acid. In certain aspects, a nucleic acid and an excipient may be a dsRNA and an excipient.

The present disclosure provides for, and includes, compositions having from 0.1 to 10 mg/ml of one ore more anti-parasitic, anti-pest or insecticidal nucleic acids. In other aspects, a composition may comprise from 0.1 to 1.0 mg/ml, 0.1 to 2.0 mg/ml, 0.1 to 2.5 mg/ml, 0.1 to 5 mg/ml, 0.1 to 10 mg/ml, 0.1 to 15 mg/ml, or 0.1 to 20 mg/ml nucleic acid. In certain aspects, a composition may comprise at least 0.1 μg/ml nucleic acid. In certain other aspects, a composition may comprise at least 1.0 μg/ml nucleic acid. In yet other aspects, a composition may comprise at least 10 μg/ml nucleic acid. In an aspect, a composition may comprise from 0.5 to 10 mg/ml nucleic acid. In other aspects, a composition may comprise from 0.5 to 1.0 mg/ml, 0.5 to 2.0 mg/ml, 0.5 to 2.5 mg/ml, 0.5 to 5 mg/ml, 0.5 to 10 mg/ml, 0.5 to 15 mg/ml, or 0.5 to 20 mg/ml nucleic acid. In an aspect, a composition may comprise from 1.0 to 10 mg/ml nucleic acid. In other aspects, a composition may comprise from 1.0 to 2.0 mg/ml, 1.0 to 2.5 mg/ml, 1.0 to 5 mg/ml, 1.0 to 10 mg/ml, 1.0 to 15 mg/ml, or 1.0 to 20 mg/ml nucleic acid. In certain aspects, the anti-parasitic, anti-pest or insecticidal nucleic acid in the composition comprises a dsRNA.

The present disclosure, provides for, and includes selective insecticide compositions and methods of using selective insecticide compositions.

As used herein, a “selective insecticide composition,” is a composition that is more effective for one or more arthropod species and is less effective for one or more different arthropod species. A selective insecticide composition includes compositions that kill adults or immature arthropods and includes compositions that are larvicides and ovicides. A selective insecticide may be a systemic insecticides incorporated by treated food, including the blood or hemolymph obtained from a host organisms. A selective insecticide may be a contact insecticides are toxic to certain insects brought into direct contact, and are non-toxic or minimally toxic to certain other insects. In some embodiments, a selective insecticide composition is anti-pest. In some embodiments, a selective insecticide composition is anti-parasitic. In some embodiments, a selective insecticide composition is a miticide. In some embodiments, a selective insecticide composition is toxic to a targeted parasitic or pest insect and non-toxic or minimally toxic to non-target organisms. Examples of non-target organisms include, but are not limited to beneficial insects, nematodes, birds, mammals, and plants. In some embodiments, a selective insecticide composition is toxic to a parasitic insect, for example Varroa mite, and non-toxic or minimally toxic to the host organism, for example bees. In some embodiments, a selective insecticide composition is toxic to one or more pest or parasitic insects selected from the group consisting of: Varroa destructor, Ixodes scapularis, Solenopsis invicta, Tetranychus urticae, Aedes aegypti, Culex quinquefasciatus, Acyrthosiphon pisum, and Pediculus humanus.

In certain aspects according to the present disclosure, a selective insecticide may be incorporated into a bacteria or yeast by genetic modification (for example, a transgenic bacteria or yeast engineered to express a nucleic acid of the present disclosure). A selective insecticide introduced by genetic modification of a bacteria or yeast may act directly on the pest organism, or indirectly by being ingested by a host of the pest organism.

In an aspect according to the present disclosure, a selective insecticide may be a more effective insecticide against one or more first insects than against one or more second insects. In an aspect, a selective insecticide may be toxic to a first insect and have no effect on a second insect. In an aspect, a selective insecticide may be toxic to a first insect and require significantly higher concentrations or amounts to have an effect on a second insect. In an aspect, a selective insecticide may be 2 times or more toxic to a first insect compared to a second insect. In an aspect, a selective insecticide may be 4 times or more toxic to a first insect compared to a second insect. In an aspect, a selective insecticide may be 5 times or more toxic to a first insect compared to a second insect. In an aspect, a selective insecticide may be 10 times or more toxic to a first insect compared to a second insect.

In an aspect, a selective insecticide may inhibit the growth, development or fecundity of a first insect and have no effect on a second insect. In an aspect, a selective insecticide may inhibit the growth, development or fecundity a first insect and require significantly higher concentrations or amounts to have a similar effect on a second insect. In an aspect, a selective insecticide may require 2 times or more of the active ingredient to inhibit the growth, development or fecundity of a second insect. In an aspect, a selective insecticide may require 4 times or more of the active ingredient to inhibit the growth, development or fecundity of a second insect. In an aspect, a selective insecticide may require 5 times or more of the active ingredient to inhibit the growth, development or fecundity of a second insect. In an aspect, a selective insecticide may require 10 times or more of the active ingredient to inhibit the growth, development or fecundity of a second insect.

Several embodiments relate to a method of altering an anti-parasitic, anti-pest or insecticidal nucleic acid molecule to reduce or eliminate regions of sequence complentarity or identity to a gene sequence of a non-target organism. In some embodiments, potential anti-parasitic, anti-pest or insecticidal nucleic acid sequences are compared to an appropriate genomic database of one or more non-target organisms (non-limited examples of non-target organisms include human, bee, monarch butterfly, mouse, rat etc.) using any sequence alignment software, such as the BLAST software available from the NCBI server (available on the internet at www.ncbi.nlm.nih.gov/BLAST/). In some embodiments, putative anti-parasitic, anti-pest or insecticidal nucleic acid molecules which exhibit significant homology to gene sequences of one or more non-target organisms are filtered out. In some embodiments, the nucleotide sequence of putative anti-parasitic, anti-pest or insecticidal nucleic acid molecules which exhibit regions of sequence complentarity or identity to a gene sequence of a non-target organism are changes in order to reduce, eliminate or disrupt such regions of sequence complentarity or identity. In some embodiments, one or more nucleotides are changed, added, or deleted compared to the target sequence to disrupt sequences having 5, 6, 7, 8, 9, 10, 11, 12, 13, 14, 15, 16, 17, 18, 19, 20, 21, 22, 23, 24, 25, 26, 27, 28, 29, 30 or more contiguous nucleotides that are identical or complementary to a gene sequence of a non-target organism. In some embodiments, 1, 2, 3, 4, 5, 6, 7, 8, 9, 10 or more nucleotides are changed, added, or deleted compared to the target sequence such that the anti-parasitic, anti-pest or insecticidal nucleic acid molecule does not comprise a sequence of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more contiguous nucleotides that are identical or complementary to a gene sequence of a non-target organism.

The present disclosure further includes, and provides for, methods of treating or preventing Colony Collapse Disorder in a honeybee colony, comprising providing an effective amount of a composition comprising a nucleic acid that is essentially identical or essentially complementary to a region of a Varroa destructor calmodulin gene sequence to a honeybee whereby the level of Varroa destructor infestation is reduced. In an aspect, the method comprises providing an effective amount of a composition comprising a nucleic acid that is essentially identical or essentially complementary to at least 19 contiguous nucleotides of SEQ ID NO: 1 and does not comprise a sequence of 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism. In an aspect, the method comprises providing an effective amount of a composition comprising a nucleic acid that is essentially identical or essentially complementary to at least 19 contiguous nucleotides of SEQ ID NO: 2 and does not comprise a sequence of 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism. In an aspect, the method comprises providing an effective amount of a composition comprising a nucleic acid that is essentially identical or essentially complementary to at least 19 contiguous nucleotides of SEQ ID NO: 69 and does not comprise a sequence of 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism. In an aspect, the method comprises providing an effective amount of a composition comprising a nucleic acid that is essentially identical or essentially complementary to at least 19 contiguous nucleotides of SEQ ID NO: 70 and does not comprise a sequence of 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism. In an aspect, the method comprises providing an effective amount of a composition comprising a nucleic acid according to SEQ ID NO: 3 and does not comprise a sequence of 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism. In an aspect, the method comprises providing an effective amount of a composition comprising a nucleic acid according to SEQ ID NO: 4 and does not comprise a sequence of 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism. In an aspect, the method comprises providing an effective amount of a composition comprising a nucleic acid according to SEQ ID NO: 88 and does not comprise a sequence of 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism. In an aspect, the method comprises providing an effective amount of a composition comprising a nucleic acid according to SEQ ID NO: 89 and does not comprise a sequence of 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism. In an aspect, the method comprises providing an effective amount of a composition comprising a nucleic acid according to SEQ ID NO: 90. In an aspect, the method comprises providing an effective amount of a composition comprising a nucleic acid according to SEQ ID NO: 91. In an aspect, the method comprises providing an effective amount of a composition comprising a nucleic acid according to SEQ ID NO: 92. In an aspect, the method comprises providing an effective amount of a composition comprising a nucleic acid according to SEQ ID NO: 93. In an aspect, the method comprises providing an effective amount of a composition comprising a nucleic acid according to SEQ ID NO: 94. In an aspect, the method comprises providing an effective amount of a composition comprising a nucleic acid according to SEQ ID NO: 90 and a nucleic acid that is the reverse complement of SEQ ID NO: 90. In an aspect, the method comprises providing an effective amount of a composition comprising a nucleic acid according to SEQ ID NO: 91 and a nucleic acid that is the reverse complement of SEQ ID NO: 91. In an aspect, the method comprises providing an effective amount of a composition comprising a nucleic acid according to SEQ ID NO: 92 and a nucleic acid that is the reverse complement of SEQ ID NO: 92. In an aspect, the method comprises providing an effective amount of a composition comprising a nucleic acid according to SEQ ID NO: 93 and a nucleic acid that is the reverse complement of SEQ ID NO: 93. In an aspect, the method comprises providing an effective amount of a composition comprising a nucleic acid according to SEQ ID NO: 94 and a nucleic acid that is the reverse complement of SEQ ID NO: 94. In an aspect, the method comprises providing an effective amount of a composition comprising two or more nucleic acids having a sequence selected from the group consisting of: SEQ ID NOs: 3, 4, 88, 89, 90-93. In an aspect, the method comprises providing an effective amount of a composition comprising a nucleic acid that is essentially identical or essentially complementary to at least 19 contiguous nucleotides of a sequence selected from SEQ ID NOs: 71-87 and does not comprise a sequence of 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism. In an aspect, the method comprises providing an effective amount of a composition comprising a nucleic acid that is essentially identical or essentially complementary to at least 23 contiguous nucleotides of a sequence selected from SEQ ID NOs: 71-87 and does not comprise a sequence of 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism. In an aspect, the method comprises providing an effective amount of a composition comprising a nucleic acid that is essentially identical or essentially complementary to at least 30 contiguous nucleotides of a sequence selected from SEQ ID NOs: 71-87 and does not comprise a sequence of 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism. In an aspect, the method comprises providing an effective amount of a composition comprising a nucleic acid that is essentially identical or essentially complementary to at least 40 contiguous nucleotides of a sequence selected from SEQ ID NOs: 71-87 and does not comprise a sequence of 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism. In an aspect, the method comprises providing an effective amount of a composition comprising a nucleic acid that is essentially identical or essentially complementary to at least 50 contiguous nucleotides of a sequence selected from SEQ ID NOs: 71-87 and does not comprise a sequence of 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism. In an aspect, the method comprises providing an effective amount of a composition comprising a nucleic acid that is essentially identical or essentially complementary to at least 60 contiguous nucleotides of a sequence selected from SEQ ID NOs: 71-87 and does not comprise a sequence of 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism. In an aspect, the method comprises providing an effective amount of a composition comprising a nucleic acid that is essentially identical or essentially complementary to at least 70 contiguous nucleotides of a sequence selected from SEQ ID NOs: 71-87 and does not comprise a sequence of 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism. In an aspect, the method comprises providing an effective amount of a composition comprising a nucleic acid that is essentially identical or essentially complementary to at least 80 contiguous nucleotides of a sequence selected from SEQ ID NOs: 71-87 and does not comprise a sequence of 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism. In an aspect, the method comprises providing an effective amount of a composition comprising a nucleic acid that is essentially identical or essentially complementary to at least 90 contiguous nucleotides of a sequence selected from SEQ ID NOs: 71-87 and does not comprise a sequence of 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism. In an aspect, the method comprises providing an effective amount of a composition comprising a nucleic acid that is essentially identical or essentially complementary to at least 100 contiguous nucleotides of a sequence selected from SEQ ID NOs: 71-87 and does not comprise a sequence of 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism. In an aspect, the method comprises providing an effective amount of a composition comprising a nucleic acid that is essentially identical or essentially complementary to at least 110 contiguous nucleotides of a sequence selected from SEQ ID NOs: 71-87 and does not comprise a sequence of 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism. In an aspect, the method comprises providing an effective amount of a composition comprising a nucleic acid that is essentially identical or essentially complementary to at least 120 contiguous nucleotides of a sequence selected from SEQ ID NOs: 71-87 and does not comprise a sequence of 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism. In an aspect, the method comprises providing an effective amount of a composition comprising a nucleic acid that is essentially identical or essentially complementary to at least 130 contiguous nucleotides of a sequence selected from SEQ ID NOs: 71-87 and does not comprise a sequence of 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism. In an aspect, the method comprises providing an effective amount of a composition comprising a nucleic acid that is essentially identical or essentially complementary to at least 140 contiguous nucleotides of a sequence selected from SEQ ID NOs: 71-87 and does not comprise a sequence of 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism. In an aspect, the method comprises providing an effective amount of a composition comprising a nucleic acid that is essentially identical or essentially complementary to a sequence selected from SEQ ID NOs: 71-87. In an aspect, the method comprises providing an effective amount of a composition comprising a nucleic acid according to a sequence selected from SEQ ID NOs: 71-87. In an aspect, the method comprises providing an effective amount of a composition comprising a nucleic acid according to a sequence selected from SEQ ID NOs: 90-94.

The present disclosure provides for, and includes, methods for reducing the parasite load of a host organism. In an aspect, the parasite load refers to the number of parasites per individual host. In an aspect, the parasite load refers to the average number of parasites per 100 host organisms. In an aspect, the parasite load may refer to the number of parasites per colony of parasite hosts. In aspects according to the present disclosure the parasite is Varroa destructor and the host is the honey bee, Apis mellifera. In certain aspects, the parasite load refers to the number of Varroa destructor parasites per 100 honeybees in a colony. In some embodiments, the present disclosure provides for, and includes, methods and compositions for reducing the parasite load to less than 6 Varroa destructor parasites per 100 honeybees in a colony. In some embodiments, the present disclosure provides for, and includes, methods and compositions for reducing the parasite load to less than 5 Varroa destructor parasites per 100 honeybees in a colony. In some embodiments, the present disclosure provides for, and includes, methods and compositions for reducing the parasite load to less than 4 Varroa destructor parasites per 100 honeybees in a colony. In some embodiments, the present disclosure provides for, and includes, methods and compositions for reducing the parasite load to less than 2 Varroa destructor parasites per 100 honeybees in a colony.

In an aspect, the methods of reducing a parasite load comprises providing an effective amount of an anti-parasitic, anti-pest or insecticidal nucleic acid composition to a host organism. An effective amount of a composition of the present disclosure results in a decrease in the parasite load over a period of time. In an aspect, a decrease in parasite load may measured within one day of providing an effective amount of a nucleic acid composition. In an aspect, the parasite load may be measured after two days. In an aspect, the parasite load may be measured after 3 days. In other aspects, the parasite load may be measured after 5 days or after 1 week. In another aspect, the parasite load may be measured more than one time, for example every 3 days, every 5 days, every week or once a month. In certain aspects, according to the present disclosure, a decrease in the number of parasites may be measured and compared to an untreated control organism or colony. In aspects according to the present disclosure the parasite is Varroa destructor and the host is the honey bee, Apis mellifera.

In aspects according to the present disclosure, a reduction in parasite load after a period of time means a decrease in the number of parasites. In an aspect, the number of parasites may decrease by 10%, 20%, 30% or more between measurements. In another aspect, the number of parasites may decrease by 40% or more between measurements. In another aspect, the number of parasites may decrease by 50% or more between measurements. In another aspect, the number of parasites may decrease by 60% or more between measurements. In another aspect, the number of parasites may decrease by 70% or more between measurements. In another aspect, the number of parasites may decrease by 80% or more between measurements. In another aspect, the number of parasites may decrease by 90% or more between measurements.

In other aspects, the parasite load may be measured as the average number of parasites per host organism. In an aspect, a decreased parasitic load may comprise fewer than 20 parasites per 100 host organisms. In an aspect, a decreased parasitic load may comprise fewer than 15 parasites per 100 host organisms. In an aspect, a decreased parasitic load may comprise fewer than 10 parasites per 100 host organisms. In an aspect, a decreased parasitic load may comprise fewer than 5 parasites per 100 host organisms. In an aspect, a decreased parasitic load may comprise fewer than 4 parasites per 100 host organisms. In an aspect, a decreased parasitic load may comprise fewer than 3 parasites per 100 host organisms. In an aspect, a decreased parasitic load may comprise fewer than 2 parasites per 100 host organisms. In an aspect, a decreased parasitic load may comprise fewer than 1 parasite per 100 host organisms. In an aspect, a decreased parasitic load may comprise fewer than 20 parasites per 1000 host organisms. In an aspect, a decreased parasitic load may comprise fewer than 15 parasites per 1000 host organisms. In an aspect, a decreased parasitic load may comprise fewer than 10 parasites per 1000 host organisms. In an aspect, a decreased parasitic load may comprise fewer than 5 parasites per 1000 host organisms. In an aspect, a decreased parasitic load may comprise fewer than 4 parasites per 1000 host organisms. In an aspect, a decreased parasitic load may comprise fewer than 3 parasites per 1000 host organisms. In an aspect, a decreased parasitic load may comprise fewer than 2 parasites per 1000 host organisms. In an aspect, a decreased parasitic load may comprise fewer than 1 parasite per 1000 host organisms.

In aspects according to the present disclosure, a colony of host organisms has an initial parasite load, prior to being provided a source of an effective amount of a nucleic acid. In an aspect, an initial parasite load may comprise fewer than 20 parasites per 100 host organisms. In an aspect, an initial parasite load may comprise fewer than 15 parasites per 100 host organisms. In an aspect, an initial parasite load may comprise fewer than 10 parasites per 100 host organisms. In an aspect, an initial parasite load may comprise fewer than 5 parasites per 100 host organisms. In an aspect, an initial parasite load may comprise fewer than 4 parasites per 100 host organisms. In an aspect, an initial parasite load may comprise fewer than 3 parasites per 100 host organisms. In an aspect, an initial parasite load may comprise fewer than 2 parasites per 100 host organisms. In an aspect, an initial parasite load may comprise fewer than 1 parasite per 100 host organisms. In aspects according to the present disclosure, an effective amount may be provided periodically or continually. In an aspect, an effective amount of an anti-parasitic, anti-pest or insecticidal nucleic acid composition may be provided once, twice or three times a day. In other aspects, an effective amount of an anti-parasitic, anti-pest or insecticidal nucleic acid composition may be provided once a day. In another aspect, an effective amount of an anti-parasitic, anti-pest or insecticidal nucleic acid composition may be provided one or more times every other day. In an aspect, an effective amount of an anti-parasitic, anti-pest or insecticidal nucleic acid composition may be provided every two days, every three days, or once a week. In an aspect, an effective amount of an anti-parasitic, anti-pest or insecticidal nucleic acid composition may be provided every two weeks. In an aspect, an effective amount of an anti-parasitic, anti-pest or insecticidal nucleic acid composition may be provided every three weeks. In an aspect, an effective amount of an anti-parasitic, anti-pest or insecticidal nucleic acid composition may be provided once a month. In an aspect, an effective amount of an anti-parasitic, anti-pest or insecticidal nucleic acid composition may be provided every two months. In an aspect, an effective amount of a nucleic acid composition may be provided continuously to an organism in need, for example by providing a continuous source of food. In one aspect, an effective amount of a nucleic acid composition may be provided continuously as a bee-ingestible composition. In aspects according to the present disclosure the parasite is Varroa destructor and the host is the honey bee, Apis mellifera. In aspects according to the present disclosure, an anti-parasitic, anti-pest or insecticidal nucleic acid may be a dsRNA.

In aspects according to the present disclosure, the parasitic load may decrease over a period of time. In an aspect, the time period necessary for a parasitic load decrease may be 15 weeks. In another aspect, the time period for a parasitic load decrease may be 12 weeks. In an aspect, the parasitic load decrease occurs of a period of 10 weeks. In an aspect, the time period necessary for a parasitic load decrease may be 5 weeks. In another aspect, the time period for a parasitic load decrease may be 2 weeks. In an aspect, the parasitic load decrease occurs of a period of 1 weeks. In some aspects, the parasitic load may decrease after one day, two days or three days.

The present disclosure provides for methods of reducing the parasitation of a honey bee colony comprising providing a bee colony an effective amount of an anti-parasitic, anti-pest or insecticidal nucleic acid composition. An effective amount of a composition of the present disclosure results in a reduction of parasitation over a period of time. In an aspect, a reduction of parasitation may be measured within one day of providing an effective amount of an anti-parasitic, anti-pest or insecticidal nucleic acid composition. In an aspect, the reduction of parasitation may be measured after two days. In an aspect, the reduction of parasitation may be measured after 3 days. In other aspects, the reduction of parasitation may be measured after 5 days or after 1 week. In another aspect, the reduction of parasitation may be measured more than one time, for example every 3 days, every 5 days, every week or once a month. In certain aspects, according to the present disclosure, a reduction of parasitation may be measured and compared to an untreated control organism or colony.

In aspects according to the present disclosure, a reduction of parasitation after a period of time means a decrease in the total number of parasites. In an aspect, the number of parasites may decrease by 10%, 20%, 30% or more between measurements. In another aspect, the number of parasites may decrease by 40% or more between measurements. In another aspect, the number of parasites may decrease by 50% or more between measurements. In another aspect, the number of parasites may decrease by 60% or more between measurements. In another aspect, the number of parasites may decrease by 70% or more between measurements. In another aspect, the number of parasites may decrease by 80% or more between measurements. In another aspect, the number of parasites may decrease by 90% or more between measurements.

In other aspects, reduction of parasitation may be measured as the average number of parasites per host organism. In an aspect, a reduction of parasitation may comprise fewer than 20 parasites per 100 host organisms. In an aspect, a reduction of parasitation may comprise fewer than 15 parasites per 100 host organisms. In an aspect, a reduction of parasitation may comprise fewer than 10 parasites per 100 host organisms. In an aspect, a reduction of parasitation may comprise fewer than 5 parasites per 100 host organisms. In an aspect, a reduction of parasitation may comprise fewer than 4 parasites per 100 host organisms. In an aspect, a reduction of parasitation may comprise fewer than 3 parasites per 100 host organisms. In an aspect, a reduction of parasitation may comprise fewer than 2 parasites per 100 host organisms. In an aspect, a reduction of parasitation may comprise fewer than 1 parasite per 100 host organisms.

In aspects according to the present disclosure, an effective amount of an anti-parasitic, anti-pest or insecticidal nucleic acid resulting in a reduction of parasitation may be provided periodically or continually. In an aspect, an effective amount of a nucleic acid composition may be provided once, twice or three times a day. In other aspects, an effective amount of an anti-parasitic, anti-pest or insecticidal nucleic acid composition may be provided once a day. In another aspect, an effective amount of an anti-parasitic, anti-pest or insecticidal nucleic acid composition may be provided one or more times every other day. In an aspect, an effective amount of an anti-parasitic, anti-pest or insecticidal nucleic acid composition may be provided provide every two days, every three days, or once a week. In an aspect, an effective amount of an anti-parasitic, anti-pest or insecticidal nucleic acid composition may be provided continuously to an organism in need, for example by providing a continuous source of food. In one aspect, an effective amount of an anti-parasitic, anti-pest or insecticidal nucleic acid composition may be provided continuously as a bee-ingestible composition. In aspects according to the present disclosure the parasite is Varroa destructor and the host is the honey bee, Apis mellifera. In aspects according to the present disclosure, an anti-parasitic, anti-pest or insecticidal nucleic acid may be a dsRNA.

In aspects according to the present disclosure, the reduction of parasitation may decrease over a period of time. In an aspect, the time period necessary for a reduction of parasitation may be 15 weeks. In another aspect, the time period for a reduction of parasitation may be 12 weeks. In an aspect, the reduction of parasitation occurs of a period of 10 weeks. In an aspect, the time period necessary for a reduction of parasitation may be 5 weeks. In another aspect, the time period for a reduction of parasitation may be 2 weeks. In an aspect, the reduction of parasitation occurs of a period of 1 weeks. In some aspects, the reduction of parasitation may occur after one day, two days or three days.

In aspects according to the present disclosure, a reduction of parasitation is measured by the number of surviving parasites as compared to an initial measurement of the number of parasites in a colony of host organisms. In an aspect, the parasite may be a Varroa destructor mite and the host may be a honey bee, Apis mellifera. In an aspect, the number of surviving parasites may be 25% of the initial number of parasites. In an aspect, the number of surviving parasites may be 15% of the initial number of parasites. In an aspect, the number of surviving parasites may be 10% of the initial number of parasites. In an aspect, the number of surviving parasites may be 5% of the initial number of parasites. In an aspect the number of surviving parasites may be less than 5% or even undetectable after providing a host colony an effective amount of an anti-parasitic, anti-pest or insecticidal nucleic acid composition.

In an aspect, the present disclosure provides for methods and compositions for reducing the susceptibility of bees to Varroa mite infestation. In other aspects, the present disclosure provides for methods and compositions to prevent the infestation of colonies of bees. In another aspect, the present disclosure provides methods and compositions for reducing the parasitation of honeybees by the mite Varroa destructor.

According to the present disclosure, a host organism provided with a source of an anti-parasitic, anti-pest or insecticidal nucleic acid, can accumulate nucleic acid in the host body, usually the hemolymph. By harboring nucleic acid, such host organisms become resistant, or less susceptible to parasitation. In other aspects, a colony of host organisms, provided with a source of nucleic acid, can accumulate nucleic acid in the host body of multiple members of the colony, thereby providing resistance or decreased susceptibility to a parasite. nucleic acid found in host organisms provided with a source of nucleic acid, can be detected using methods known to those of ordinary skill in the art. In aspects according to the present disclosure, an anti-parasitic, anti-pest or insecticidal nucleic acid may be a dsRNA.

In an aspect of the present disclosure, methods and compositions for treating Varroa mite infestations in bees by down-regulating calmodulin and calmodulin related Varroa mite gene products, are provided. In an aspect, the compositions comprise an anti-parasitic, anti-pest or insecticidal nucleic acid corresponding to the Varroa destructor calmodulin sequence of SEQ ID NO: 1. In an aspect, the compositions comprise an anti-parasitic, anti-pest or insecticidal nucleic acid corresponding to the Varroa destructor calmodulin sequence of SEQ ID NO: 2. In an aspect, the compositions comprise an anti-parasitic, anti-pest or insecticidal nucleic acid corresponding to the Varroa destructor calmodulin sequence of SEQ ID NO: 69. In an aspect, the compositions comprise an anti-parasitic, anti-pest or insecticidal nucleic acid corresponding to the Varroa destructor calmodulin sequence of SEQ ID NO: 70. In some aspects, the compositions comprise an anti-parasitic, anti-pest or insecticidal nucleic acid corresponding to a Varroa destructor calmodulin sequence selected from SEQ ID NOs: 71-87. In another aspect, the compositions comprise an anti-parasitic, anti-pest or insecticidal nucleic acid corresponding to a sequence selected from SEQ ID NOs: 3, 4, and 88-93. In an aspect, the compositions comprise a small RNA corresponding to the Varroa destructor calmodulin sequence of SEQ ID NO: 1. In an aspect, the compositions comprise a small RNA corresponding to the Varroa destructor calmodulin sequence of SEQ ID NO: 2. In an aspect, the compositions comprise a small RNA corresponding to the Varroa destructor calmodulin sequence of SEQ ID NO: 69. In an aspect, the compositions comprise a small RNA corresponding to the Varroa destructor calmodulin sequence of SEQ ID NO: 70. In some aspects, the compositions comprise a small RNA corresponding to a Varroa destructor calmodulin sequence selected from SEQ ID NOs: 71-87. In another aspect, the compositions comprise a small RNA corresponding to a Varroa destructor calmodulin sequence selected from SEQ ID NOs: 3, 4, 88 and 89. In another aspect, the compositions comprise a small RNA corresponding to a sequence selected from SEQ ID NOs: 90-93. In an aspect, the compositions comprise a dsRNA corresponding to the Varroa destructor calmodulin sequence of SEQ ID NO: 1. In an aspect, the compositions comprise a dsRNA corresponding to the Varroa destructor calmodulin sequence of SEQ ID NO: 2. In an aspect, the compositions comprise a dsRNA corresponding to the Varroa destructor calmodulin sequence of SEQ ID NO: 69. In an aspect, the compositions comprise a dsRNA corresponding to the Varroa destructor calmodulin sequence of SEQ ID NO: 70. In some aspects, the compositions comprise a dsRNA corresponding to a Varroa destructor calmodulin sequence selected from SEQ ID NOs: 71-87. In another aspect, the compositions comprise a dsRNA corresponding to a Varroa destructor calmodulin sequence selected from SEQ ID NOs: 3, 4, 88 and 89. In another aspect, the compositions comprise a dsRNA comprising a sequence selected from SEQ ID NOs: 90-93. In an aspect, the compositions comprise an siRNA corresponding to the Varroa destructor calmodulin sequence of SEQ ID NO: 1. In an aspect, the compositions comprise a siRNA corresponding to the Varroa destructor calmodulin sequence of SEQ ID NO: 2. In an aspect, the compositions comprise a siRNA corresponding to the Varroa destructor calmodulin sequence of SEQ ID NO: 69. In an aspect, the compositions comprise a siRNA corresponding to the Varroa destructor calmodulin sequence of SEQ ID NO: 70. In some aspects, the compositions comprise a siRNA corresponding to a Varroa destructor calmodulin sequence selected from SEQ ID NOs: 71-87. In another aspect, the compositions comprise a siRNA corresponding to a Varroa destructor calmodulin sequence selected from SEQ ID NOs: 3, 4, 88 and 89. In another aspect, the compositions comprise a siRNA corresponding to a sequence selected from SEQ ID NOs: 90-93. In aspects according to the present disclosure the composition may comprise an anti-parasitic, anti-pest or insecticidal nucleic acid corresponding to a region of SEQ ID NO: 1 or 2. In other aspects according to the present disclosure the composition may comprise an anti-parasitic, anti-pest or insecticidal nucleic acid corresponding to a region of SEQ ID NO: 69 or 70. In yet other aspects according to the present disclosure the composition may comprise a nucleic acid corresponding to a region of a sequence selected from SEQ ID NOs: 3, 4, 88 and 89. In yet other aspects according to the present disclosure the composition may comprise a nucleic acid corresponding to a region of a sequence selected from SEQ ID NOs: 90-94.

Varroa mites parasitize pupae and adult bees and reproduce in the pupal brood cells. The mites use their mouths to puncture the exoskeleton and feed on the bee's hemolymph. The present inventors unexpectedly found that polynucleotide agents administered to the bees to treat Varroa mite infestations presented in the bee's hemolymph thereby becoming available to the mite.

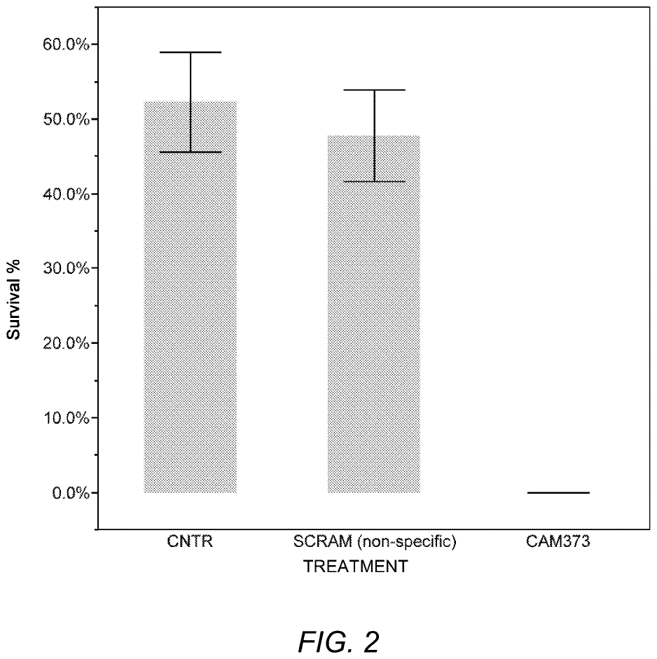

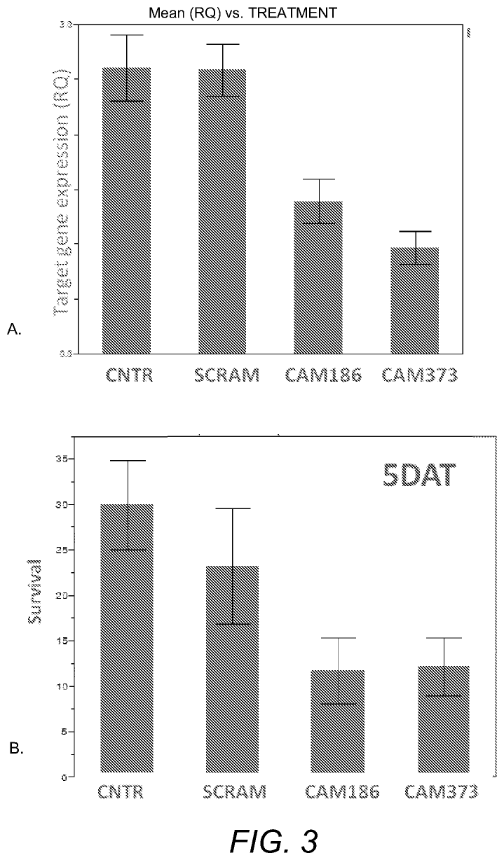

The present inventors have shown that calmodulin-targeting dsRNA fragments can successfully be transferred to Varroa mites (see, e.g., FIG. 2), that the dsRNA can serve to down-regulate expression of calmodulin genes in the Varroa mite (see, e.g., FIG. 3 Panel A) and further that targeting of calmodulin genes for down-regulation can result in a reduction in the number of Varroa mites (see, e.g., FIG. 3 Panel B).

Thus, according to one aspect of the present disclosure there is provided a method of preventing or treating a Varroa destructor mite infestation of a bee, the method comprising administering to the bee an effective amount of a nucleic acid agent comprising a nucleic acid sequence which downregulates expression of a calmodulin gene of a Varroa destructor mite, thereby preventing or treating a Varroa destructor mite infestation of a bee.

According to this aspect of the present disclosure the agents of the present disclosure are used to prevent the Varroa destructor mite from living as a parasite on the bee, or larvae thereof. The phrase “Varroa destructor mite” refers to the external parasitic mite that attacks honey bees Apis cerana and Apis mellifera. The mite may be at an adult stage, feeding off the bee, or at a larval stage, inside the honey bee brood cell.

As mentioned, the agents of the present disclosure are capable of selectively down-regulating expression of a gene product of a Varroa destructor mite. As used herein, the phrase “gene product” refers to an RNA molecule or a protein. According to one aspect, the Varroa destructor mite gene product is one which is essential for mite viability. Down-regulation of such a gene product would typically result in killing of the Varroa mite. According to another aspect, the Varroa destructor mite gene product is one which is essential for mite reproduction. Down-regulation of such a gene product would typically result in the prevention of reproduction of the Varroa mite and the eventual extermination of the mite population. According to yet another aspect, the Varroa destructor mite gene product is one which is required to generate pathogenic symptoms in the bee. In some aspects, the Varroa destructor gene product is a calmodulin gene. In certain aspects, the calmodulin gene may comprise a nucleic acid sequence according to SEQ ID NO: 1 or SEQ ID NO: 2. In certain aspects, the calmodulin gene may comprise a nucleic acid sequence according to SEQ ID NO: 69 or SEQ ID NO: 70.

Examples of gene products that may be down-regulated according to this aspect of the present disclosure include, but are not limited to a calmodulin gene.

In an aspect according to the present disclosure, agents capable of down-regulating expression of a gene product of a Varroa destructor mite or other parasite, may downregulate to a lesser extent expression of the gene product in other animals, such as the bee or other non-target organism. Accordingly, certain agents of the present disclosure are able to distinguish between the mite gene and the bee gene, down-regulating the former to a greater extent than the latter. In some aspects, certain agents of the present disclosure are able to distinguish between the target gene in the target organism and orthologs in non-target organisms, down-regulating the former to a greater extent than the latter. In other aspects, the target gene of the parasite is downregulated while the homologous host gene is not. In yet another aspect, the target gene of the parasite does not have a homologue in the host. According to another aspect the agents of the present disclosure do not down-regulate the bee gene whatsoever. For example, this may be effected by targeting a gene that is expressed differentially in the mite and not in the bee e.g. the mite sodium channel gene—FJ216963. Alternatively, the agents of the present disclosure may be targeted to mite-specific sequences of a gene that is expressed both in the mite and in the bee.

According to one aspect, the agents of the present disclosure target segments of Varroa genes that are at least 100 bases long and do not carry any sequence longer than 5 bases that is entirely homologous to any bee-genome sequence or human-genome sequence. According to one aspect, the agents of the present disclosure target segments of Varroa genes that are at least 100 bases long and do not carry any sequence longer than 6 bases that is entirely homologous to any bee-genome sequence or human-genome sequence. According to one aspect, the agents of the present disclosure target segments of Varroa genes that are at least 100 bases long and do not carry any sequence longer than 7 bases that is entirely homologous to any bee-genome sequence or human-genome sequence. According to one aspect, the agents of the present disclosure target segments of Varroa genes that are at least 100 bases long and do not carry any sequence longer than 8 bases that is entirely homologous to any bee-genome sequence or human-genome sequence. According to one aspect, the agents of the present disclosure target segments of Varroa genes that are at least 100 bases long and do not carry any sequence longer than 9 bases that is entirely homologous to any bee-genome sequence or human-genome sequence. According to one aspect, the agents of the present disclosure target segments of Varroa genes that are at least 100 bases long and do not carry any sequence longer than 10 bases that is entirely homologous to any bee-genome sequence or human-genome sequence. According to one aspect, the agents of the present disclosure target segments of Varroa genes that are at least 100 bases long and do not carry any sequence longer than 11 bases that is entirely homologous to any bee-genome sequence or human-genome sequence. According to one aspect, the agents of the present disclosure target segments of Varroa genes that are at least 100 bases long and do not carry any sequence longer than 12 bases that is entirely homologous to any bee-genome sequence or human-genome sequence. According to one aspect, the agents of the present disclosure target segments of Varroa genes that are at least 100 bases long and do not carry any sequence longer than 13 bases that is entirely homologous to any bee-genome sequence or human-genome sequence. According to one aspect, the agents of the present disclosure target segments of Varroa genes that are at least 100 bases long and do not carry any sequence longer than 14 bases that is entirely homologous to any bee-genome sequence or human-genome sequence. According to one aspect, the agents of the present disclosure target segments of Varroa genes that are at least 100 bases long and do not carry any sequence longer than 15 bases that is entirely homologous to any bee-genome sequence or human-genome sequence. According to one aspect, the agents of the present disclosure target segments of Varroa genes that are at least 100 bases long and do not carry any sequence longer than 16 bases that is entirely homologous to any bee-genome sequence or human-genome sequence. According to one aspect, the agents of the present disclosure target segments of Varroa genes that are at least 100 bases long and do not carry any sequence longer than 17 bases that is entirely homologous to any bee-genome sequence or human-genome sequence. According to one aspect, the agents of the present disclosure target segments of Varroa genes that are at least 100 bases long and do not carry any sequence longer than 18 bases that is entirely homologous to any bee-genome sequence or human-genome sequence. According to one aspect, the agents of the present disclosure target segments of Varroa genes that are at least 100 bases long and do not carry any sequence longer than 19 bases that is entirely homologous to any bee-genome sequence or human-genome sequence. According to one aspect, the agents of the present disclosure target segments of Varroa genes that are at least 100 bases long and do not carry any sequence longer than 20 bases that is entirely homologous to any bee-genome sequence or human-genome sequence. According to one aspect, the agents of the present disclosure target segments of Varroa genes that are at least 100 bases long and do not carry any sequence longer than 21 bases that is entirely homologous to any bee-genome sequence or human-genome sequence. According to one aspect, the agents of the present disclosure target segments of Varroa genes that are at least 100 bases long and do not carry any sequence longer than 22 bases that is entirely homologous to any bee-genome sequence or human-genome sequence. According to one aspect, the agents of the present disclosure target segments of Varroa genes that are at least 100 bases long and do not carry any sequence longer than 23 bases that is entirely homologous to any bee-genome sequence or human-genome sequence. According to one aspect, the agents of the present disclosure target segments of Varroa genes that are at least 100 bases long and do not carry any sequence longer than 24 bases that is entirely homologous to any bee-genome sequence or human-genome sequence. According to one aspect, the agents of the present disclosure target segments of Varroa genes that are at least 100 bases long and do not carry any sequence longer than 25 bases that is entirely homologous to any bee-genome sequence or human-genome sequence. According to one aspect, the agents of the present disclosure target segments of Varroa genes that are at least 100 bases long and do not carry any sequence longer than 26 bases that is entirely homologous to any bee-genome sequence or human-genome sequence. According to one aspect, the agents of the present disclosure target segments of Varroa genes that are at least 100 bases long and do not carry any sequence longer than 27 bases that is entirely homologous to any bee-genome sequence or human-genome sequence. According to one aspect, the agents of the present disclosure target segments of Varroa genes that are at least 100 bases long and do not carry any sequence longer than 28 bases that is entirely homologous to any bee-genome sequence or human-genome sequence. According to one aspect, the agents of the present disclosure target segments of Varroa genes that are at least 100 bases long and do not carry any sequence longer than 29 bases that is entirely homologous to any bee-genome sequence or human-genome sequence. According to one aspect, the agents of the present disclosure target segments of Varroa genes that are at least 100 bases long and do not carry any sequence longer than 30 bases that is entirely homologous to any bee-genome sequence or human-genome sequence. While it will be appreciated that more than one gene may be targeted in order to maximize the cytotoxic effect on the Varroa mites, compositions that comprise one, or a few, small RNA's would increase the probability of being a selective insecticide composition as cross reactivity with other insects may be reduced.

According to one aspect, a dsRNA composition can be prepared corresponding to the Varroa destructor Calmodulin-1 and Calmodulin-2 genes (e.g. using nucleic acid agents having the sequence as set forth in SEQ ID NOs: 1 to 4, and 69 to 89, their complements or nucleic acids directed to regions thereof).

It will be appreciated that as well as down-regulating a number of genes, the present disclosure further provides for, and includes, using a number of agents to down-regulate the same gene (e.g. a number of nucleic acids, or dsRNAs, each hybridizing to a different segment of the same gene). For example, in an aspect a combination of one or more nucleic acids corresponding to a sequence selected from the group consisting of SEQ ID NOs: 1 to 4, 6, 23, 26 to 35, and 69 to 89 may be used to increase the cytotoxic and anti-parasitic effects of the composition. Tools which are capable of identifying species-specific sequences may be used for this purpose, e.g. BLASTN and other such computer programs. U.S. Patent Publication NOs. 20090118214 and 20120108497 provide for the use of dsRNA for preventing and treating viral infections in honeybees. U.S. Patent Publication Nos. 20120258646 provides for the use of dsRNA to control Varroa destructor in honeybee. Each publication is hereby incorporated in their entireties.

The present disclosure provides for, and includes, compositions and methods for down-regulating the expression of a gene in a target organism. In an aspect the target organism may be a parasite. In certain aspects, the parasite may be Varroa destructor. As used herein, the term “down-regulating expression” refers to causing, directly or indirectly, reduction in the transcription of a desired gene, reduction in the amount, stability or translatability of transcription products (e.g. RNA) of the gene, and/or reduction in translation of the polypeptide(s) encoded by the desired gene. Down-regulating expression of a gene product of a Varroa destructor mite can be monitored, for example, by direct detection of gene transcripts (for example, by PCR), by detection of polypeptide(s) encoded by the gene or bee pathogen RNA (for example, by Western blot or immunoprecipitation), by detection of biological activity of polypeptides encode by the gene (for example, catalytic activity, ligand binding, and the like), or by monitoring changes in the Varroa destructor mite (for example, reduced proliferation of the mite, reduced virulence of the mite, reduced motility of the mite etc) and by testing bee infectivity/pathogenicity.

Downregulation of a pest or parasite gene product can be effected on the genomic and/or the transcript level using a variety of agents which interfere with transcription and/or translation (e.g., RNA silencing agents, Ribozyme, DNAzyme and antisense nucleic acid molecules). Downregulation of a Varroa destructor mite gene product can be effected on the genomic and/or the transcript level using a variety of agents which interfere with transcription and/or translation (e.g., RNA silencing agents, Ribozyme, DNAzyme and antisense nucleic acid molecules).

According to one aspect, the agent which down-regulates expression of a pest or parasite gene product is a small RNA, such as an RNA silencing agent. According to this aspect, the small RNA is greater than 15 base pairs in length. In another aspect, the small RNA is greater than 50 base pairs in length. In an aspect, the small RNA is greater than 50 base pairs in length but less than about 500 base pairs. In an aspect, the small RNA is greater than 100 base pairs in length but less than about 500 base pairs. In an aspect, the small RNA is greater than 200 base pairs in length but less than about 500 base pairs. In an aspect, the pest or parasite may be a Varroa destructor mite.

Another method of down-regulating a pest or parasite gene product is by introduction of small inhibitory RNAs (siRNAs). Another method of down-regulating a Varroa mite gene product is by introduction of small inhibitory RNAs (siRNAs).

In one aspect of the present disclosure, synthesis of RNA silencing agents suitable for use with the present disclosure can be effected as follows. First, the pest or parasite target mRNA is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex (Tuschl ChemBiochem. 2:239-245). It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (available on the internet at www.ambion.com/techlib/tn/91/912.html).

Second, potential target sites are compared to an appropriate genomic database of non-target organisms (e.g., human, bee, monarch butterfly, mouse, rat etc.) using any sequence alignment software, such as the BLAST software available from the NCBI server (available on the internet at www.ncbi.nlm.nih.gov/BLAST/). In some embodiments, Putative target sites which exhibit significant homology to other coding sequences are filtered out. In some embodiments, one or more nucleotides are changed or deleted compared to the target sequence to disrupt sequences having 5, 6, 7, 8, 9, 10, 11, 12, 13, 14, 15, 16, 17, 18, 19, 20, 21, 22, 23, 24, 25 or more contiguous nucleotides that are 100% identical or complementary to a gene sequence of a non-target organism.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene or sequence for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene or pest or parasite target sequence. An example of a scrambled nucleotide sequence is provided at SEQ ID NO. 5.

For example, a siRNA that may be used in this aspect of the present disclosure is one which targets a mite-specific calmodulin gene. Examples of siRNAs are provided in SEQ ID NOs: 3, 4, 88 and 89.

It will be appreciated that the RNA silencing agent of the present disclosure need not be limited to those molecules containing only RNA, but further encompasses chemically-modified nucleotides and non-nucleotides.

In some aspects, the RNA silencing agent provided herein can be functionally associated with a cell-penetrating peptide. As used herein, a “cell-penetrating peptide” is a peptide that comprises a short (about 12-residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of the present disclosure preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of the present disclosure preferably include, but are not limited to, penetratin, transportan, plsl, TAT (48-60), pVEC, MTS, and MAP.

Another agent capable of down-regulating a pest or parasite gene product is a DNAzyme molecule capable of specifically cleaving an mRNA transcript or DNA sequence of the bee pathogen polypeptide. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine: pyrimidine junctions (Santoro, S. W. & Joyce, G. F. Proc. Natl, Acad. Sci. USA 199; for a review of DNAzymes, see Khachigian, L M, Curr Opin Mol Ther 4:119-21 (2002)). In an aspect, the pest or parasite gene product may be a Varroa mite gene product. Downregulation of pest or parasite gene products can also be effected by using an antisense polynucleotide capable of specifically hybridizing with an mRNA transcript encoding the pest or parasite gene product. Design of antisense molecules which can be used to efficiently downregulate a pest or parasite gene product must be effected while considering two aspects important to the antisense approach. The first aspect is delivery of the oligonucleotide into the cytoplasm of the appropriate cells, while the second aspect is design of an oligonucleotide which specifically binds the designated mRNA or RNA target sequence within cells in a way which inhibits translation thereof. In an aspect, the pest or parasite gene product may be a Varroa mite gene product. In another aspect, the pest or parasite gene product may be calmodulin gene product.

A number of delivery strategies which can be used to efficiently deliver oligonucleotides into a wide variety of cell types (see, for example, Luft J Mol Med 76:75-6 (1998); Kronenwett et al. Blood 91:852-62 (1998); Rajur et al. Bioconjug Chem 8:935-40 (1997); Lavigne et al. Biochem Biophys Res Commun 237:566-71 (1997) and Aoki et al. (1997) Biochem Biophys Res Commun 231:540-5 (1997)).

In addition, algorithms for identifying those sequences with the highest predicted binding affinity for their target mRNA based on a thermodynamic cycle that accounts for the energetics of structural alterations in both the target mRNA and the oligonucleotide are also available (see, for example, Walton et al. Biotechnol Bioeng 65:1-9 (1999)). Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gpl) in cell culture as evaluated by a kinetic PCR technique proved effective in almost all cases, including tests against three different targets in two cell types with phosphodiester and phosphorothioate oligonucleotide chemistries. In addition, several approaches for designing and predicting efficiency of specific oligonucleotides using an in vitro system were also published (Matveeva et al., Nature Biotechnology 16:1374-1375 (1998)].

Another agent capable of down-regulating a pest or parasite gene product is a ribozyme molecule capable of specifically cleaving an mRNA transcript encoding the Varroa mite gene product. Ribozymes are being increasingly used for the sequence-specific inhibition of gene expression by the cleavage of mRNAs encoding proteins of interest (Welch et al., Curr Opin Biotechnol. 9:486-96 (1998)). The possibility of designing ribozymes to cleave any specific target RNA, including viral RNA, has rendered them valuable tools in both basic research and therapeutic applications. In an aspect, the pest or parasite gene product may be a Varroa mite gene product. In another aspect, the pest or parasite gene product may be calmodulin gene product.

An additional method of down-regulating the expression of a pest or parasite gene product in cells is via triplex forming oligonucleotides (TFOs). Recent studies have shown that TFOs can be designed which can recognize and bind to polypurine/polypyrimidine regions in double-stranded helical DNA in a sequence-specific manner. These recognition rules are outlined by Maher III, L. J., et al., Science (1989) 245:725-7; Moser, H. E., et al., Science, (1987) 238:645-6; Beal, P. A., et al., Science (1992) 251:1360-1363; Cooney, M., et al., Science (1988) 241:456-459; and Hogan, M. E., et al., EP Publication 375408. Modification of the oligonucleotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 112:487-94). In an aspect, the pest or parasite gene product may be a Varroa mite gene product. In another aspect, the pest or parasite gene product may be calmodulin gene product.

In general, the triplex-forming oligonucleotide has the sequence correspondence:

| | |
|---|---|
| oligo | 3′--A G G T |
| duplex | 5′--A G C T |
| duplex | 3′--T C G A |

However, it has been shown that the A-AT and G-GC triplets have the greatest triple helical stability (Reither and Jeltsch, BMC Biochem, 2002 September 12, Epub). The same authors have demonstrated that TFOs designed according to the A-AT and G-GC rule do not form non-specific triplexes, indicating that the triplex formation is indeed sequence specific.

Triplex-forming oligonucleotides preferably are at least 15, more preferably 25, still more preferably or more nucleotides in length, up to 50 or 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression.

Detailed description of the design, synthesis and administration of effective TFOs can be found in U.S. Patent Publication Nos. 2003/017068 and 2003/0096980 to Froehler et al., and 2002/0128218 and 2002/0123476 to Emanuele et al., and U.S. Pat. No. 5,721,138 to Lawn.

The polynucleotide down-regulating agents of the present disclosure may be generated according to any polynucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the polynucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

The polynucleotide agents of the present disclosure may comprise heterocylic nucleosides consisting of purines and the pyrimidines bases, bonded in a 3′ to 5′ 5phosphodiester linkage.

Preferably used polynucleotide agents are those modified in either backbone, internucleoside linkages or bases, as is broadly described hereinunder.

Specific examples of polynucleotide agents useful according to this aspect of the present disclosure include polynucleotide agents containing modified backbones or non-natural internucleoside linkages. Polynucleotide agents having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Modified polynucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,216,141; 5,235,033; 5,264,562; 5,264,564; 5,405,938; 5,470,967; 5,489,677; 5,541,307; 5,561,225; 5,596,086; 5,602,240; 5,602,240; 5,608,046; 5,610,289; 5,618,704; 5,623,070; 5,663,312; 5,214,134; 5,466,677; 5,610,289; 5,633,360; 5,677,437; and 5,677,439.

Other polynucleotide agents which can be used according to the present disclosure, are those modified in both sugar and the internucleoside linkage, i.e., the backbone, of the nucleotide units are replaced with novel groups. The base units are maintained for complementation with the appropriate polynucleotide target. An example for such an polynucleotide mimetic, includes peptide nucleic acid (PNA). A PNA polynucleotide refers to a polynucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present disclosure are disclosed in U.S. Pat. No. 6,303,374.

Polynucleotide agents of the present disclosure may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 613, and those disclosed by Sanghvi, Y. S., Chapter 15, Antisense Research and Applications, pages 289-2, Crooke, S. T. and Lebleu, B., ed., CRC Press, 1993. Such bases are particularly useful for increasing the binding affinity of the oligomeric compounds of the disclosure. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi Y S et al. (1993) Antisense Research and Applications, CRC Press, Boca Raton 276-278) and are presently preferred base substitutions, even more particularly when combined with 2′-O-methoxyethyl sugar modifications.

Following synthesis, the polynucleotide agents of the present disclosure may optionally be purified. For example, polynucleotides can be purified from a mixture by extraction with a solvent or resin, precipitation, electrophoresis, chromatography, or a combination thereof. Alternatively, polynucleotides may be used with no, or a minimum of, purification to avoid losses due to sample processing. The polynucleotides may be dried for storage or dissolved in an aqueous solution. The solution may contain buffers or salts to promote annealing, and/or stabilization of the duplex strands.

It will be appreciated that a polynucleotide agent of the present disclosure may be provided per se, or as a nucleic acid construct comprising a nucleic acid sequence encoding the polynucleotide agent. Typically, the nucleic acid construct comprises a promoter sequence which is functional in the host cell, as detailed herein below.

The polynucleotide sequences of the present disclosure, under the control of an operably linked promoter sequence, may further be flanked by additional sequences that advantageously affect its transcription and/or the stability of a resulting transcript. Such sequences are generally located upstream of the promoter and/or downstream of the 3′ end of the expression construct.

The term “operably linked”, as used in reference to a regulatory sequence and a structural nucleotide sequence, means that the regulatory sequence causes regulated expression of the linked structural nucleotide sequence. “Regulatory sequences” or “control elements” refer to nucleotide sequences located upstream, within, or downstream of a structural nucleotide sequence, and which influence the timing and level or amount of transcription, RNA processing or stability, or translation of the associated structural nucleotide sequence. Regulatory sequences may include promoters, translation leader sequences, introns, enhancers, stem-loop structures, repressor binding sequences, termination sequences, pausing sequences, polyadenylation recognition sequences, and the like.

It will be appreciated that the nucleic acid agents can be delivered to the pest or parasite in a great variety of ways. According to one aspect, the nucleic acid agents are delivered directly to the pest or parasite (e.g. by spraying a mite infested hive). The nucleic acid agents, or constructs encoding same may enter the mites bodies by diffusion. In this aspect, the promoter of the nucleic acid construct is typically operational in mite cells. In an aspect, the pest or parasite may be Varroa destructor.

It will be appreciated that since many parasites use their mouths to puncture the host arthropod exoskeleton and feed on the arthropod's hemolymph, the present disclosure contemplates delivering the polynucleotide agents of the present disclosure to the arthropod, whereby they become presented in the arthropod hemolymph thereby becoming available to the pest or parasite. Thus, according to another aspect, the nucleic acid agents are delivered indirectly to the pest or parasite (for example to a mite via a host bee). In this aspect, the promoter of the nucleic acid construct is typically operational in host cells. In certain aspects, the pest or parasite may be Varroa destructor and the host arthropod may be a bee.

According to one aspect, the nucleic acid agents are delivered to the infested hosts by spraying. The nucleic acid agents, or constructs encoding same may enter the host's bodies by diffusion.

In certain aspects, the pest or parasite may be Varroa destructor and the host arthropod may be a bee.

According to another aspect, the nucleic acid agents are delivered to the host via its food. The present inventors consider that following ingestion of the nucleic acid agents of the present disclosure, the agents can be presented, for example in a host arthropod in the host's hemolymph, whereby it becomes available to the parasite, for example a Varroa mite.

Thus the polynucleotides of the present disclosure may be synthesized in vitro or in vivo, for example in a bacterial or yeast cell, and added to the food. For example double stranded RNA may be synthesized by adding two opposing promoters (e.g. T7 promoters) to the ends of the gene segments, wherein the promoter is placed immediately 5′ to the gene and the promoter is placed immediately 3′ to the gene segment in the opposite orientation. The dsRNA may then be prepared by transcribing in vitro with the T7 RNA polymerase.

Examples of sequences for synthesizing nucleic acids, including dsRNA, according to aspects of the present disclosure are provided in SEQ ID NOs: 1 to 4, 6, 23, 26 to 35, and 69 to 93.

It will be appreciated that some pests or parasites cause wound sites in the exoskeleton of a host arthropod. Such wound sites harbor bacterial infections. For example, a host bee wound site may harbor a bacteria such as Melissococcus pluton, which causes European foulbrood. In addition, to their parasitic effects, parasites are known to act as vectors for a number of other pathogens and parasites. For example, Varroa mites are suspected of acting as vectors for a number of honey bee pathogens, including deformed wing virus (DWV), Kashmir bee virus (KBV), acute bee paralysis virus (ABPV) and black queen cell virus (BQCV), and may weaken the immune systems of their hosts, leaving them vulnerable to infections.

Thus, by killing the pest or parasite (or preventing reproduction thereof), the anti-parasitic, anti-pest or insecticidal agents of the present disclosure may be used to prevent and/or treat bacterial infections of host organisms. For example, Melissococcus pluton and viral infections in host bees caused by the above named viruses. Since Varroa mite infestation and viral infections are thought to be responsible for colony collapse disorder (CCD), the present agents may also be used to prevent or reduce the susceptibility of a bee colony to CCD.

It will be appreciated that in addition to feeding of anti-parasitic, anti-pest or insecticidal nucleic acid agents for reduction of the bee pathogen infection and infestation, enforcement of proper sanitation (for example, refraining from reuse of infested hives) can augment the effectiveness of treatment and prevention of infections.

Also included and provided for by the present disclosure are transgenic bacteria and yeast cells that express a selective insecticide. In one aspect, a nucleic acid encoding a small RNA, dsRNA, miRNA or a small or miRNA-resistant target nucleic acid molecule used herein is operably linked to a promoter and optionally a terminator. In some embodiments, the transgenic bacteria and yeast cells are killed, for example, by applying heat or pressure. In some embodiments, the transgenic bacteria and yeast cells are lysed prior to providing the selective insecticide to the target organism. In some embodiments, the transgenic bacteria and yeast cells are not lysed.

In one aspect, an exogenous nucleic acid molecule used herein is or encodes a small RNA, or in a particular aspect a siRNA, which can modulate the expression of a gene in a target organism. In an aspect, an exogenous nucleic acid encodes a small RNA having at least 80%, 85%, 88%, 90%, 92%, 95%, 96%, 97%, 98%, or 99% sequence identity to a sequence selected from the group consisting of SEQ ID NOs: 1-4 and 6-94. In a further aspect, an exogenous nucleic acid molecule used herein is or encodes a dsRNA molecule. In another aspect, an exogenous nucleic acid molecule used herein is or encodes an artificial miRNA. In a further aspect, an exogenous nucleic acid molecule used herein is or encodes an siRNA. In one aspect, an exogenous nucleic acid molecule used herein is or encodes a precursor of a small RNA. In another aspect, an exogenous nucleic acid molecule used herein is or encodes a precursor of a miRNA or siRNA. In one aspect, an exogenous nucleic acid molecule used herein is a naturally-occurring molecule. In another aspect, an exogenous nucleic acid molecule used herein is a synthetic molecule.

In one aspect, an exogenous nucleic acid molecule used herein is or encodes a stem-loop precursor of a small RNA or in a particular aspect a miRNA, comprising a sequence having at least 80%, 85%, 88%, 90%, 92%, 95%, 96%, 97%, 98%, or 99% sequence identity to a sequence selected from the group consisting of SEQ ID NOs: 1-4 and 6-94. A stem-loop precursor used herein comprises a sequence having at least 60%, 65%, 70%, 75%, 80%, 85%, 88%, 90%, 92%, 95%, 96%, 97%, 98%, or 99% sequence identity to a sequence selected from the group consisting of SEQ ID NOs: 1-4 and 6-94.

In one aspect, an exogenous nucleic acid molecule used herein is naked RNA or expressed from a nucleic acid expression construct, where it is operably linked to a regulatory sequence.

In one aspect, a recombinant DNA construct or a transgene disclosed herein further comprises a transcription terminator.

It is expected that during the life of a patent maturing from this application many relevant methods for down-regulating expression of gene products can be developed and the scope of the term “down-regulating expression of a gene product of a Varroa destructor mite” is intended to include all such new technologies a priori.

It is appreciated that certain features of the disclosure, which are, for clarity, described in the context of separate aspects, may also be provided in combination in a single aspect. Conversely, various features of the disclosure, which are, for brevity, described in the context of a single aspect, may also be provided separately or in any suitable subcombination or as suitable in any other described aspect of the disclosure. Certain features described in the context of various aspects are not to be considered essential features of those aspects, unless the aspect is inoperative without those elements. Various aspects and aspects of the present disclosure as delineated hereinabove and as claimed in the claims section below find experimental support in the following examples.

### EXAMPLES

### Example 1. Varroa Mite Calmodulin Gene Sequences

The Calmodulin (CAM) genes provided in Table 1 (SEQ ID NO: 1 and 2), or their corresponding transcripts, were used as targets of polynucleotide compositions comprising a polynucleotide that is at least 18 contiguous nucleotides identical or complementary to those genes or transcripts. The gene sequences provided in Table 1, protein sequences encoded by those genes, or sequences contained within those genes were used to obtain orthologous Calmodulin (CAM) genes from other arthropod pest and parasitic species not listed in Table 1. Such orthologous genes and their transcripts can then serve as targets of polynucleotides provided herein or as a source of anti-parasitic, anti-pest or insecticidal polynucleotides that are specifically designed to target the orthologous genes or transcripts.

TABLE 1
Target Calmodulin (CAM) genes of Varroa destructor

| Gene | SEQ | |
|---|---|---|
| name | ID | Open reading frame DNA sequence |
| CAM- | 1 | ATGGCTGATCAGCTAACTGAGGAACAGATCGCCGAGTTCA |
| 1 | | AAGAGGCGTTTAGCCTGTTTGACAAGGACGGAGATGGCAC |
| | | GATCACGACAAAGGAGCTCGGTACGGTAATGCGATCTCTC |
| | | GGCCAGAACCCCACTGAGGCTGAACTGCAGGACATGATCA |
| | | ACGAGGTCGACGCCGACGGCTCCGGAACGATAGATTTCCC |
| | | TGAGTTCCTCACAATGATGGCAAGAAAGATGAAGGACACC |
| | | GACTCGGAGGAGGAGATCCGAGAGGCGTTCCGCGTATTCG |
| | | ACAAGGATGGCAACGGTTTCATTTCGGCGGCCGAGCTCAG |
| | | GCACGTTATGACCAACCTTGGCGAGAAGCTTACGGACGAG |
| | | GAGGTAGATGAGATGATTCGGGAGGCAGATATTGACGGTG |
| | | ATGGTCAGGTCAACTACGAGGAGTTCGTCACCATGATGAC |
| | | GTCCAAGTAAATGGCGGATCAGCTGACCGAGGAGCAAATC |
| | | GCCGAATTCAAGGAGGCTTTCAGCCTGTTCGATAAAG |
| CAM- | 2 | ACGGTGATGGCACAATTACGACCAAGGAACTAGGGACCGT |
| 2 | | CATGCGGTCCCTCGGCCAGAACCCTACTGAGGCTGAGCTT |
| | | CAAGACATGATCAACGAGGTCGACGCTGACGGTAACGGCA |
| | | CTATTGACTTTCCAGAGTTTCTCACGATGATGGCGCGTAA |
| | | AATGAAGGACACCGACTCCGAGGAGGAGATCCGGGAAGCT |
| | | TTTAGGGTTTTTGATAAAGACGGAAATGGCTTCATTTCGG |
| | | CTGCAGAGCTGAGGCACGTAATGACCAACCTTGGCGAAAA |
| | | GCTCACGGACGAGGAAGTGGACGAGATGATCCGCGAGGCG |
| | | GATATCGACGGCGACGGACAGGTCAACTACGAGGAGTTCG |
| | | TCACGATGATGACATCAAAATGA |

For each Calmodulin DNA gene sequence provided in SEQ ID NO: 1 and 2, single stranded or double stranded DNA or RNA fragments in sense or antisense orientation or both are fed in vitro to Varroa mites grown on a petri plate or applied topically to bee hives to effect the expression of the CAM target genes and obtain a reduction in Varroa destructor mite population.

### Example 2. Suppression of Calmodulin (CAM) Genes of Varroa destructor

Polynucleotides for the suppression of expression of Calmodulin (CAM) genes in Varroa destructor mite corresponding to SEQ ID NOs: 3 and 4 (Table 2) are provided and were used to used to suppress expression of Calmodulin (CAM) genes in Varroa destructor mite. The SEQ ID NOs: 3 and 4 describe a 373 bp dsRNA polynucleotide sequence and a 186 bp dsRNA polynucleotide sequence, respectively, selected from CAM-1 (SEQ ID NO: 1). SEQ ID NO: 3, corresponding to dsRNA polynucleotide CAM_L/CAM373 covers most of the open reading frame of the Calmodulin CAM-1 (SEQ ID NO: 1) gene. SEQ ID NO 4, corresponding to dsRNA polynucleotide CAM_S/CAM186 is a partial fragment of CAM_L/CAM373 (SEQ ID NO: 3) and is also derived from CAM-1 (SEQ ID NO: 1). SEQ ID NO: 5 in Table 2 is a control dsRNA sequence polynucleotide sequence with no more than 19 bp sequence identity to any known Varroa destructor gene.

TABLE 2
dsRNAs targeting Varroa destructor Calmodulin
(CAM) genes

| dsRNA | SEQ | |
|---|---|---|
| name | ID | Nucleic acid sequence |
| CAM_ | 3 | ACAGAUCGCCGAGUUCAAAGAGGCGUUUAGCCUGUU |
| L/CAM | | UGACAAGGACGGAGAUGGCACGAUCACGACAAAGGA |
| 373 | | GCUCGGUACGGUAAUGCGAUCUCUCGGCCAGAACCC |
| | | CACUGAGGCUGAACUGCAGGACAUGAUCAACGAGGU |
| | | CGACGCCGACGGCUCCGGAACGAUAGAUUUCCCUGA |
| | | GUUCCUCACAAUGAUGGCAAGAAAGAUGAAGGACAC |
| | | CGACUCGGAGGAGGAGAUCCGAGAGGCGUUCCGCGU |
| | | AUUCGACAAGGAUGGCAACGGUUUCAUUUCGGCGGC |
| | | CGAGCUCAGGCACGUUAUGACCAACCUUGGCGAGAA |
| | | GCUUACGGACGAGGAGGUAGAUGAGAUGAUUCGGGA |
| | | GGCAGAUAUUGAC |
| CAM_ | 4 | ACAAUGAUGGCAAGAAAGAUGAAGGACACCGACUCG |
| S/CAM | | GAGGAGGAGAUCCGAGAGGCGUUCCGCGUAUUCGAC |
| 186 | | AAGGAUGGCAACGGUUUCAUUUCGGCGGCCGAGCUC |
| | | AGGCACGUUAUGACCAACCUUGGCGAGAAGCUUACG |
| | | GACGAGGAGGUAGAUGAGAUGAUUCGGGAGGCAGAU |
| | | AUUGAC |
| SCRAM | 5 | AUACUUACUGGUGCUAAUUUUUAUCGAGGAUGCCCA |
| | | ACUCCCCCCACUUUAAAACUGCGAUCAUACUAACGA |
| | | ACUCCCGAAGGAGUGAAAGGUGUCUAUGUUGAGCUU |
| | | AAUAACCUACCUUGCGAGCAAAGAAGGACUAGUUGA |
| | | CCCUGGGCACCCUAUAUUGUUAUGUUGUUUCGAACU |
| | | GAGUUGGCACCCAUGCUGCACAUGCAACAAACAUGU |
| | | CGGCCUUCGUGUCUAUCCUAGAAAAGUACCUGUGAA |
| | | CUUGGCUGUCUACAUCAUCAUC |

### Example 3. Varroa destructor Bioassay at 3 Day Post-Treatment with Specific dsRNAs

Adult female mites were collected from honeybee colonies and placed in a petri dish plate on top of an artificial diet solution containing a mixture of 1% tryptone, 0.5% yeast extract, 1% NaCl and 15 mg/mL agar. In this example the diet was supplemented with 50 μg kanamycin per 1 mL of diet solution. The diet/agar solution was further supplemented with 200-500 μg/mL of dsRNA and the resulting solution was poured on a petri dish. The dsRNA in this example consisted of either SEQ ID NO: 3 (CAM_L/CAM373) or SEQ ID NO: 5 (SCRAM). Fifteen mites were applied to each plate and the experiment was conducted in triplicate. The diet plates with the mites were incubated at 29° C. with 50-60% relative humidity. At specific time intervals the plates were inspected and dead mites were counted and removed. For mortality studies the mites were counted three days after being placed on the diet (FIG. 2). FIG. 2 shows that all mites were dead at three day after treatment compared to untreated plates or plates where the mites were fed on a diet supplemented with the non-specific (SCRAM) dsRNA polynucleotide.

### Example 4. Varroa destructor Bioassay at 5 Day Post-Treatment with dsRNAs Targeting Calmodulin

Adult female mites were collected from honeybee colonies and placed in a petri dish plate on top of an artificial diet solution. The artificial diet contained a mixture of 1% tryptone, 0.5% yeast extract, 1% NaCl and 15 mg/mL agar. In this example the diet was further supplemented with Antimycotic Solution (100×, Sigma Aldrich) at 8× final concentration, 500 μg/mL kanamycin and 220 U/mL nystatin. The diet/agar solution was further supplemented with 200-500 μg/mL of dsRNA and the resulting solution was poured on a petri dish. The dsRNA in this example consisted of either SEQ ID NO: 3 (CAM_L/CAM373), or SEQ ID NO: 4 (CAM_S/CAM186), or SEQ ID NO: 5 (SCRAM). Fifteen mites were applied to each plate and the experiment was conducted in triplicate. The diet plates with the mites were incubated at 29° C. with 50-60% relative humidity. At specific time intervals the plates were inspected and dead mites were counted and removed. For mortality studies the mites were counted at five days after being placed on the diet (FIG. 3). For molecular analysis, live mites were removed from the plates, snap frozen in liquid nitrogen and TAQMAN™ analysis was performed to assess the levels of Calmodulin (CAM) RNA. FIG. 3, Panel A. the RNA levels for Calmodulin (CAM) genes in mites exposed to SEQ ID NO: 3 (CAM_L/CAM373) or SEQ ID NO: 4 (CAM_S/CAM186) was highly reduced compared to the non-specific (SCRAM) treatment or no treatment (CNTR). FIG. 3, Panel B, a statistically significant mortality in mites that were exposed to dsRNA against Calmodulin (CAM) was observed at 5 days after treatment.

### Example 5. Method for Delivering of dsRNA Polynucleotides Targeting Varroa Genes Using a Spray-Dried or Semi-Solid Formulation

dsRNA used to suppress expression of Varroa target Calmodulin (CAM) genes was prepared in a formulation containing 1 part dsRNA and ~14 parts trehalose in a phosphate buffer (a solution of 1.15 mM KH₂PO₄ (monobasic) and 8 mM Na₂HPO₄ (dibasic), pH 8.0) as illustrated in Table 3. Using a Büchi B-290 mini spray dryer, the liquid formulation was atomized into droplets and heated with gas to produce a flowable powder.

TABLE 3
Formulation Preparation

| | | | | | | | | | Ratio of AI |
|---|---|---|---|---|---|---|---|---|---|
| | Stock | Final | | | | | Active | Active | (dsRNA) |
| | buffer (X % w/v | buffer (X % w/v | Total | Stock | dsRNA | | Ingredient | Ingredient | to Buffer |
| | trehalose + | trehalose + | vol | buffer | stock | | (AI) conc | (AI) conc | (trehalose + |
| dsRNA | phosphate buffer) | phosphate buffer) | (mL) | (mL) | (mL) | Ratio | (mg/mL) | (% solids) | phosphate buffer) |
| CAM_L/CAM373 | 40 | 10 | 1100 | 275.00 | 825.00 | ¼ | 7.20 | 0.720 | 13.9 |
| CAM_S/CAM186 | 40 | 10 | 1285 | 321.21 | 963.75 | ¼ | 6.75 | 0.675 | 14.8 |

The resulting particles were formulated with powdered sugar and applied evenly to hives by spreading the powdered sugar evenly on top of the frames. In other aspects, a semi-solid preparation of the spray-dried material is prepared with water and the sugar-water (“bee-candy”) formulation is fed to the bee hives by allowing the bees to feed on it.

### Example 6. In Vivo Reduction of Varroa Mite in Bee Hives after Treatment with dsRNA Targeting Calmodulin (CAM) Genes

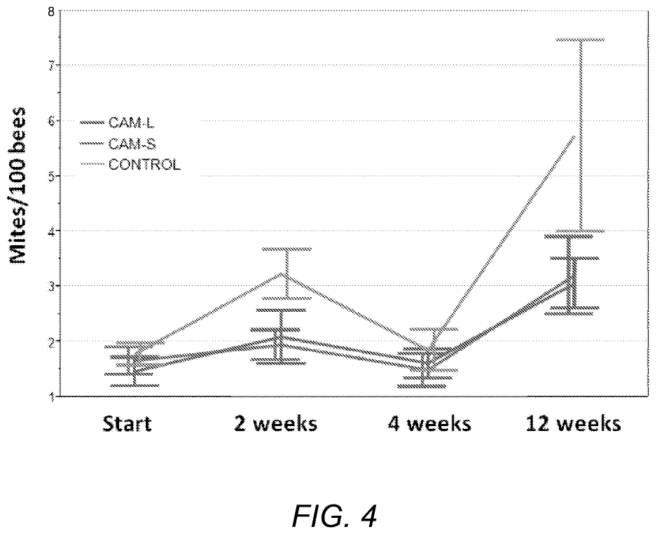

Varroa mites infesting adult honey bees in the hives were collected and counted using standard mite counting methodology. Hives were treated with spray dried dsRNA according to Example 7 comprising SEQ ID NO: 3 (CAM-L), SEQ ID NO: 4 (CAM-S), or no treatment (CONTROL). The mite load of each hive was assessed at the beginning of the experiment and at 2 weeks, 4 weeks and 12 weeks after treatment. FIG. 4 shows the mite load of the treated hives compared to the hives that did not receive the treatment. The number of mites counted was normalized to 100 adult bees and is representative of the Varroa mite load.

### Example 7. Detection of Transitive Small RNAs in Varroa Following Treatment with dsRNA Targeting Calmodulin (CAM) Genes

Varroa mites were collected from hives treated with SEQ ID NO: 3 dsRNA polynucleotides and collected from the hive at 7 day after treatment. Varroa RNA was extracted and small RNA sequencing analysis performed using the SOLID platform. The majority of small RNA molecules were detected outside the dsRNA sequence region and specifically toward the 3′ portion of the dsRNA region of SEQ ID NO: 3. Additionally, the majority of the transitive reads were in the antisense orientation relative to the Calmodulin (CAM) gene transcript sequence. Further, small RNAs specific for CAM-2 (SEQ ID NO: 2) were detected in this experiment despite the hives being treated with dsRNA for SEQ ID NO: 3, which is predicted to be specific for CAM-1 (SEQ ID NO: 1). This observation supports the hypothesis that suppression of RNA expression and transitive small RNA generation in Varroa works even when only a small fragment between the two genes shares complete identity at the DNA level (in this case 23 nucleotides).

### Example 8. Calmodulin (CAM) Gene Homologs from Arthropod Pest and Parasite Species and Corresponding dsRNA Polynucleotides

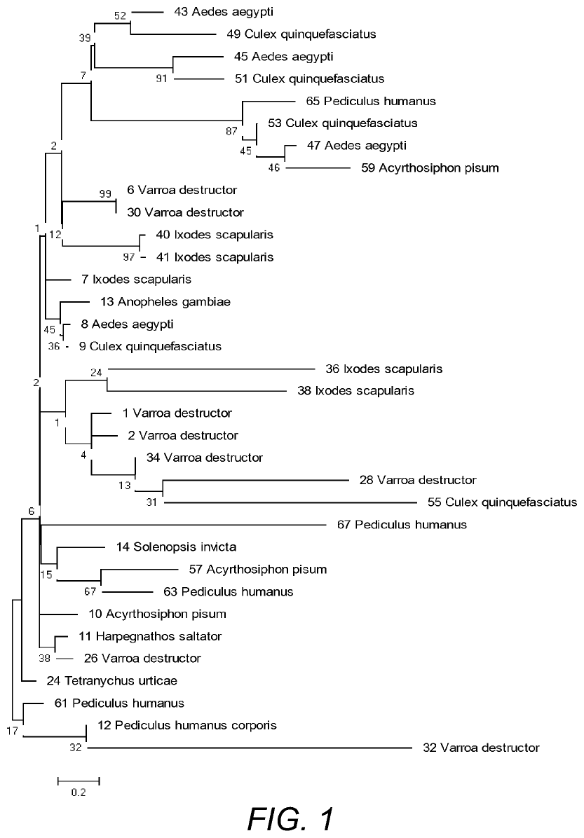

Using standard bioinformatics technique and the sequences SEQ ID NOs: 1 and 2 for Varroa destructor a set of 31 conserved Calmodulin (CAM) gene sequences were identified in arthropod pest species that infest either other arthropods or mammals and that will be targeted for gene regulation. These sequences were identified and presented as a phylogenetic tree in FIG. 1. The DNA sequences in FIG. 1 were further analyzed by identifying the conserved 373 bp domain within each sequence that corresponds to SEQ ID NO: 3 (CAM_L/CAM373). Table 4 lists the SEQ ID NOs of the newly identified Calmodulin (CAM) gene sequences as well as the corresponding 373 bp dsRNA polynucleotide trigger sequences. The 373 bp polynucleotide dsRNA sequences will be tested either alone or in combination in direct feeding assays against their respective arthropod species.

TABLE 4
Calmodulin (CAM) gene sequences identified from arthropod pests
or parasites and their corresponding 373 bp RNA polynucleotides.

| SEQ ID NO | Gene Name | Organism/Species | Type |
|---|---|---|---|
| 6 | CAM-3 | Varroa destructor | cDNA |
| 7 | CAM-1 | Ixodes scapularis | cDNA |
| 8 | CAM-1 | Aedes aegypti | cDNA |
| 9 | CAM-1 | Culex quinquefasciatus | cDNA |
| 10 | CAM-1 | Acyrthosiphon pisum | cDNA |
| 11 | CAM-1 | Harpegnathos saltator | cDNA |
| 12 | CAM-1 | Pediculus humanus corporis | cDNA |
| 13 | CAM-1 | Anopheles gambiae | cDNA |
| 14 | CAM-1 | Solenopsis invicta | cDNA |
| 15 | CAM-1 | Ixodes scapularis | RNA |
| 16 | CAM-1 | Aedes aegypti | RNA |
| 17 | CAM-1 | Culex quinquefasciatus | RNA |
| 18 | CAM-1 | Acyrthosiphon pisum | RNA |
| 19 | CAM-1 | Harpegnathos saltator | RNA |
| 20 | CAM-1 | Pediculus humanus corporis | RNA |
| 21 | CAM-1 | Anopheles gambiae | RNA |
| 22 | CAM-1 | Solenopsis invicta | RNA |
| 23 | CAM-3 | Varroa destructor | RNA |
| 24 | CAM-1 | Tetranychus urticae | cDNA |
| 25 | CAM-1 | Tetranychus urticae | RNA |
| 26 | CAM-4 | Varroa destructor | cDNA |
| 27 | CAM-4 | Varroa destructor | RNA |
| 28 | CAM-5 | Varroa destructor | cDNA |
| 29 | CAM-5 | Varroa destructor | RNA |
| 30 | CAM-7 | Varroa destructor | cDNA |
| 31 | CAM-7 | Varroa destructor | RNA |
| 32 | CAM-8 | Varroa destructor | cDNA |
| 33 | CAM-8 | Varroa destructor | RNA |
| 34 | CAM-9 | Varroa destructor | cDNA |
| 35 | CAM-9 | Varroa destructor | RNA |
| 36 | CAM | Ixodes scapularis | cDNA |
| 37 | CAM | Ixodes scapularis | RNA |
| 38 | CAM | Ixodes scapularis | cDNA |
| 39 | CAM | Ixodes scapularis | RNA |
| 40 | CAM | Ixodes scapularis | cDNA |
| 41 | CAM | Ixodes scapularis | cDNA |
| 42 | CAM | Ixodes scapularis | RNA |
| 43 | CAM | Aedes aegypti | cDNA |
| 44 | CAM | Aedes aegypti | RNA |
| 45 | CAM | Aedes aegypti | cDNA |
| 46 | CAM | Aedes aegypti | RNA |
| 47 | CAM | Aedes aegypti | cDNA |
| 48 | CAM | Aedes aegypti | RNA |
| 49 | CAM | Culex quinquefasciatus | cDNA |
| 50 | CAM | Culex quinquefasciatus | RNA |
| 51 | CAM | Culex quinquefasciatus | cDNA |
| 52 | CAM | Culex quinquefasciatus | RNA |
| 53 | CAM | Culex quinquefasciatus | cDNA |
| 54 | CAM | Culex quinquefasciatus | RNA |
| 55 | CAM | Culex quinquefasciatus | cDNA |
| 56 | CAM | Culex quinquefasciatus | RNA |
| 57 | CAM | Acyrthosiphon pisum | cDNA |
| 58 | CAM | Acyrthosiphon pisum | RNA |
| 59 | CAM | Acyrthosiphon pisum | cDNA |
| 60 | CAM | Acyrthosiphon pisum | RNA |
| 61 | CAM | Pediculus humanus | cDNA |
| 62 | CAM | Pediculus humanus | RNA |
| 63 | CAM | Pediculus humanus | cDNA |
| 64 | CAM | Pediculus humanus | RNA |
| 65 | CAM | Pediculus humanus | cDNA |
| 66 | CAM | Pediculus humanus | RNA |
| 67 | CAM | Pediculus humanus | cDNA |
| 68 | CAM | Pediculus humanus | RNA |

### Example 9. Varroa Calmodulin (CAM) Gene Transcripts and dsRNA Trigger Sequences

The Calmodulin (CAM) sequences provided in Table 5 (SEQ ID NOs: 69 and 70), or their corresponding transcripts, were used as targets of polynucleotide compositions comprising a polynucleotide that is at least 18 contiguous nucleotides identical or complementary to those genes or transcripts. The 5′ and 3′UTR sequences for the Varroa Calmodulin sequences were identified by RNA sequencing.

TABLE 5
Target transcripts for Calmodulin
(CAM) genes of Varroa destructor

| Gene name and Species | SEQ ID NO | Type |
|---|---|---|
| CAM-1; Varroa destructor | 69 | RNA |
| CAM-2; Varroa destructor | 70 | RNA |

SEQ ID NOs: 69 and 70 were tiled in 150 bp fragments. Table 6 illustrates the top strand (5′-3′) for the 150 bp fragments that tile across SEQ ID NOs: 69 and 70.

TABLE 6
Tiled polynucleotide sequences for CAM-1 and CAM-2 genes

| | | Position within |
|---|---|---|
| Gene name | SEQ ID NO | transcript sequence |
| CAM-1 | 71 | 1-150 |
| CAM-1 | 72 | 151-300 |
| CAM-1 | 73 | 301-450 |
| CAM-1 | 74 | 451-600 |
| CAM-1 | 75 | 601-750 |
| CAM-1 | 76 | 751-900 |
| CAM-1 | 77 | 901-1050 |
| CAM-1 | 78 | 1051-1200 |
| CAM-1 | 79 | 1201-1350 |
| CAM-1 | 80 | 1351-1500 |
| CAM-2 | 81 | 1-150 |
| CAM-2 | 82 | 151-300 |
| CAM-2 | 83 | 301-450 |
| CAM-2 | 84 | 451-600 |
| CAM-2 | 85 | 601-750 |
| CAM-2 | 86 | 751-900 |
| CAM-2 | 87 | 901-1050 |

One or more dsRNA comprising a sequence selected from SEQ ID NOs: 71-87 is provided in vitro to Varroa mites grown on a petri plate or applied topically to bee hives to effect the expression of the CAM target genes and obtain a reduction in Varroa destructor mite population.

### Example 10. In Vitro Bioassay of Calmodulin (CAM) Targeting Triggers in Varroa Mite

Polynucleotide trigger sequences targeting Calmodulin (CAM)-1 and 2 were generated based on conserved sequence overlap between CAM-1 and CAM-2 sequences. These are presented as SEQ ID NOs: 88 and 89 (targeting CAM-1 and CAM-2, respectively).

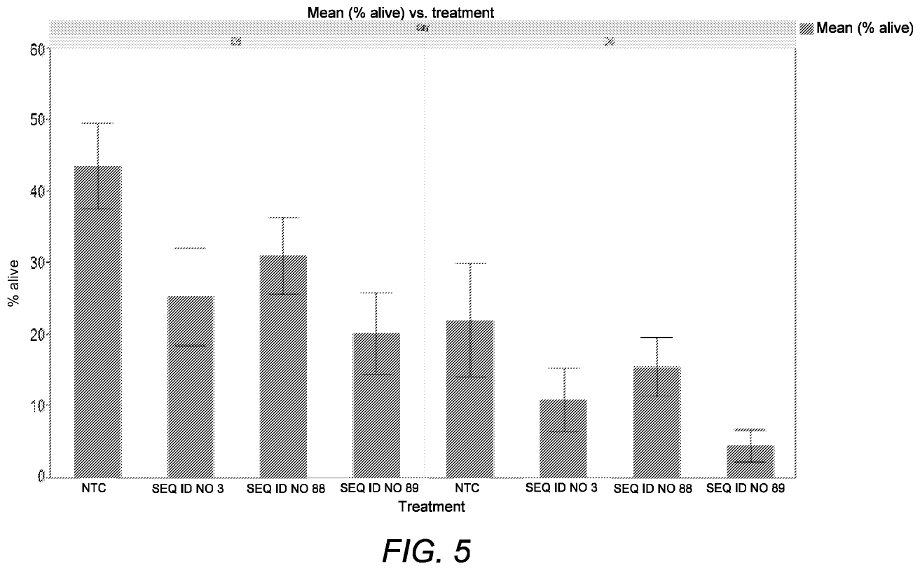

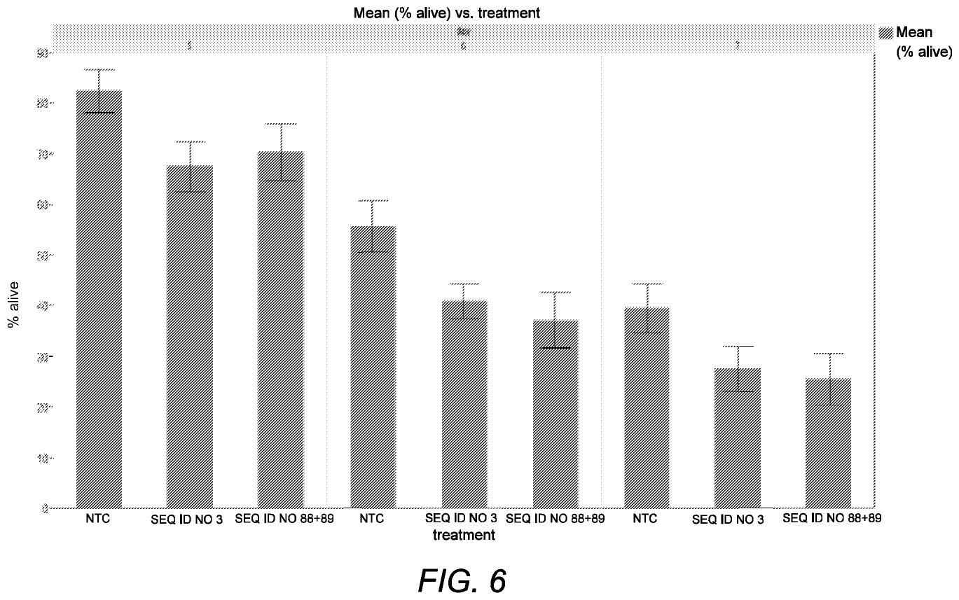

Polynucleotide sequences selected from SEQ ID NOs: 88 and 89 were tested in an in vitro bioassay for their ability to suppress viability of adult Varroa mites. Adult female mites were collected from honeybee colonies and placed in a petri dish plate on top of an artificial diet solution. The artificial diet contained a mixture of 1% tryptone, 0.5% yeast extract, 1% NaCl and 15 mg/mL agar. In this example, the diet was further supplemented with Antimycotic Solution (100×, Sigma Aldrich) at 8× final concentration, 500 μg/mL kanamycin and 220 U/mL nystatin. The diet/agar solution was further supplemented with 200-500 μg/mL of dsRNA and the resulting solution was poured on a petri dish. The dsRNA in this example consisted of either SEQ ID NO: 3 (CAM373), SEQ ID NO: 88 (CAM-1), or SEQ ID NO: 89 (CAM-2) or non-treated control (NTC). Fifteen mites were applied to each plate and the experiment was conducted in triplicate. The diet plates with the mites were incubated at 29° C. with 50-60% relative humidity. At specific time intervals the plates were inspected and dead mites were counted and removed. For mortality studies the mites were counted at five and six days after being placed on the diet (FIG. 5.). Additionally, the dsRNA for SEQ ID NO: 88 (CAM-1) and SEQ ID NO: 89 (CAM-2) were mixed in equimolar amount and fed as described above to the mites. FIG. 6 shows the result of this application.

For molecular analysis, live mites are removed from the plates, snap frozen in liquid nitrogen and TAQMAN™ analysis is performed to assess the levels of Calmodulin (CAM) RNA.

### Example 11. In Vivo Field Reduction of Varroa Mite Infestation in Field Treated Bee Hives after Treatment with dsRNA Targeting Calmodulin (CAM) Gene

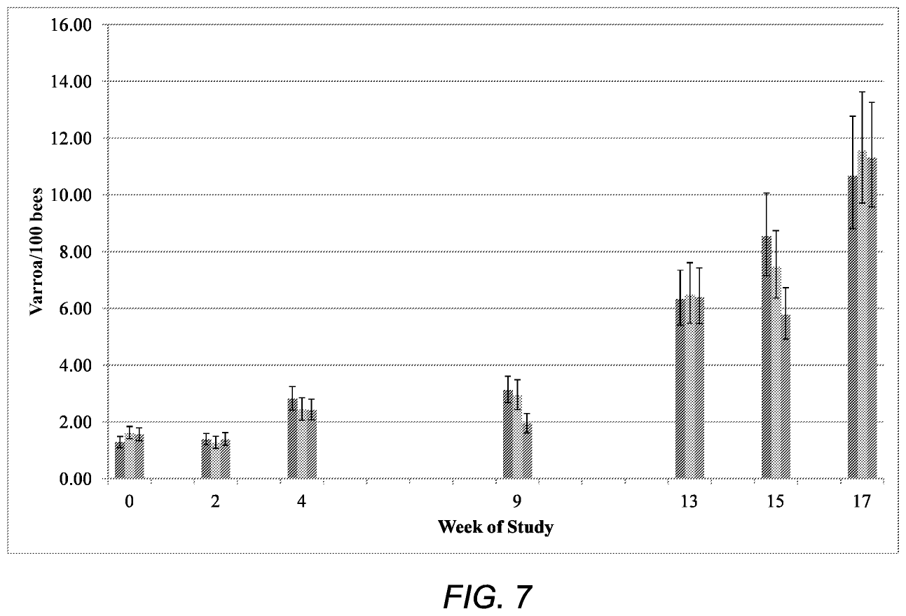

dsRNA used to suppress expression of Varroa targeted Calmodulin (CAM) genes was prepared by mixing dsRNA stock in Phosphate Buffer with 66% sugar syrup. The liquid formulation was supplied as a syrup to the bees, allowed to feed on it until fully consumed (approximately 2-3 days). Each field testing group consisted of 33 hives. The groups consisted of non-treated hives, non-specific trigger treated (SEQ ID NO: 5) and specific trigger treated (SEQ ID NO: 3). Bees were treated in two rounds, each round consisted of two feedings two weeks apart: at the start of the delivery (week 0) and two weeks later (week 2), then again on week 13 and 15. Assessment of bee survival was done at 4, 9, 13, 15 and 17 weeks (FIG. 7). Significant suppression of Varroa population was observed following treatment with the specific trigger (SEQ ID NO:3) at week 9.

### Example 12. In Vitro Bioassay of Calmodulin (CAM) Targeting Triggers in Varroa Mite

Polynucleotide trigger sequences targeting Calmodulin (CAM)-1 and 2 were generated based on conserved sequence overlap between CAM-1 and CAM-2 sequences. These are presented in Table 7 as SEQ ID NOs: 3. Additionally, the polynucleotide sequences were modified to avoid containing a consecutive sequence string of 19 bases that are identical or complementary to a gene sequence of a non-target organism. These modifications resulted in polynucleotide SEQ ID NOs: 90-92.

TABLE 7
Triggers targeting CAM-1 and CAM-2

| Gene Target | SEQ ID NO | Native or modified |
|---|---|---|
| CAM-1 | 3 | native |
| CAM-1 | 90 | 4 bp deletion |
| CAM-1 | 91 | 4 bp deletion |
| CAM-2 | 92 | 1 bp deletion |

Polynucleotide sequences were tested in an in vitro bioassay for their ability to suppress viability of adult Varroa mites. Adult female mites were collected from honeybee colonies and placed in a petri dish plate on top of an artificial diet solution. The artificial diet contained a mixture of 1% tryptone, 0.5% yeast extract, 1% NaCl and 15 mg/mL agar. In this example the diet was further supplemented with Antimycotic Solution (100×, Sigma Aldrich) at 8× final concentration, 500 μg/mL kanamycin and 220 U/mL nystatin. The diet/agar solution was further supplemented with 200-500 μg/mL of dsRNA (either SEQ ID NO: 3 (CAM373), SEQ ID NO: 90 (CAM-1), or SEQ ID NO: 92 (CAM-2)) and the resulting solution was poured on a petri dish. Plates containing the artificial diet without dsRNA were also prepared (non-treated control (NTC)). Fifteen mites were applied to each plate and the experiment was conducted in triplicate. The diet plates with the mites were incubated at 29° C. with 50-60% relative humidity. At specific time intervals the plates were inspected and dead mites were counted and removed. The mites were counted at five and six days after being placed on the diet to determine mortality.

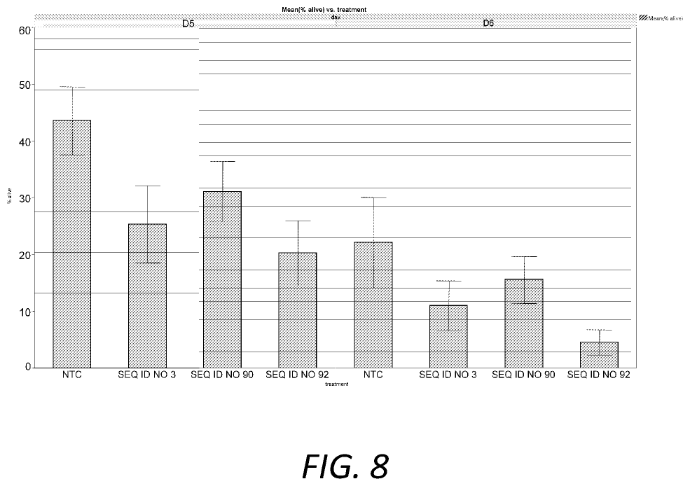

As shown in FIG. 8, mites treated with dsRNA (either SEQ ID NO: 3 (CAM373), SEQ ID NO: 90 (CAM-1), or SEQ ID NO: 92 (CAM-2) exhibited increased mortality 5 and 6 days after treatment compared to mites that did not receive RNA.

A further in vitro bioassay was performed as described above in which mites were treated with: an equimolar mixture of dsRNA comprising SEQ ID NO: 90 (CAM-1) and SEQ ID NO: 92

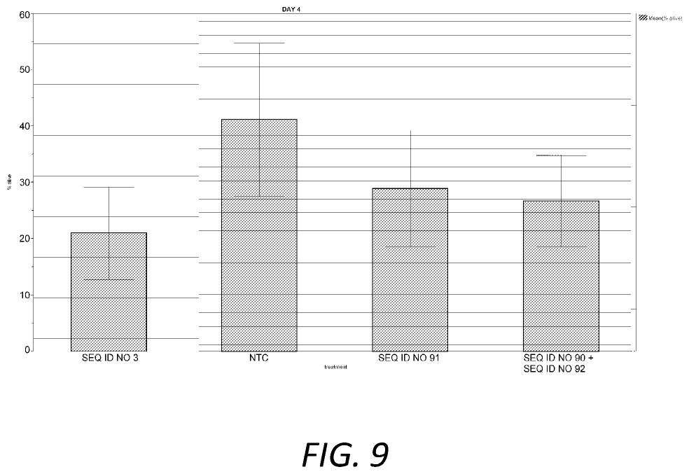

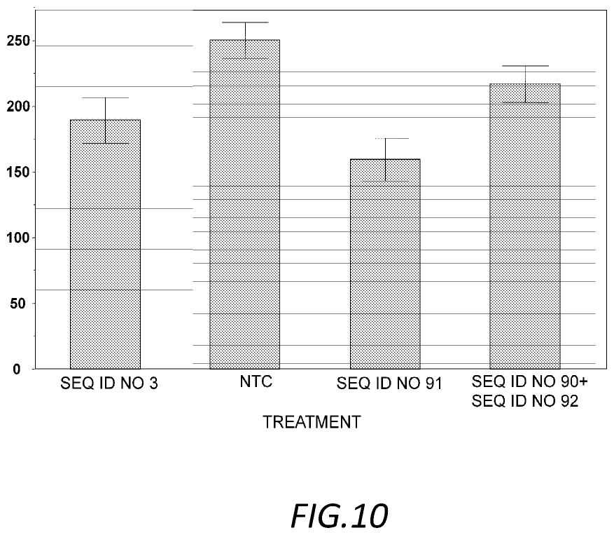

(CAM-2); dsRNA comprising SEQ ID NO: 3 (CAM373) or dsRNA comprising SEQ ID NO: 91 (CAM-1). As shown in FIG. 9, mites treated with dsRNA comprising SEQ ID NO: 3, SEQ ID NO: 91 or a mixture of dsRNAs comprising SEQ ID NO: 90 and SEQ ID NO: 92 exhibit reduced viability at Day 4 (D4). Molecular analysis was performed on live mites. Mites were removed from the plates, snap frozen in liquid nitrogen and QuantiGene™ analysis was performed to assess the levels of Calmodulin (CAM) RNA. Results are shown in FIG. 10.

### Example 13. In Vivo Field Reduction of Varroa Mite Infestation in Field Treated Bee Hives after Treatment with dsRNA Targeting Calmodulin (CAM) Gene

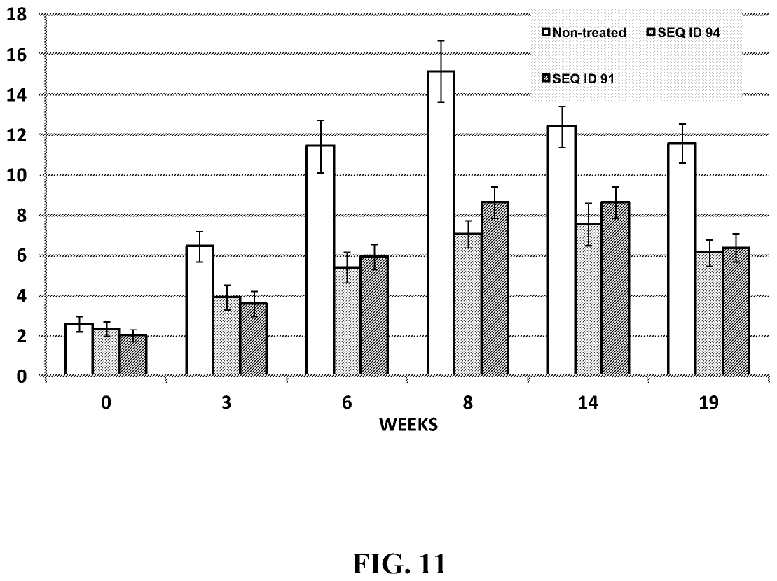

dsRNA used to suppress expression of Varroa targeted Calmodulin (CAM) genes was prepared by mixing dsRNA stock in Phosphate Buffer with 66% sugar syrup. The liquid formulation was supplied as a syrup to the bees, allowed to feed on it until fully consumes (approximately 2-3 days). Each field testing group consisted of 40 hives. The groups consisted of non-treated and specific trigger treated hives (SEQ ID NO: 91 or SEQ ID NO: 94). SEQ ID NO 94 is identical to SEQ ID NO 3 with the exception of the addition of three bases at the 5′-end of the trigger. Bees were treated in two rounds, each round consisted of two feedings two weeks apart: at the start of the delivery (week 0) and two weeks later (week 2), then again on week 13 and 15. The dosage consisted of approximately 200 mg trigger per hive. Assessment of bee survival was done at 0, 3, 6, 8, 14 and 19 weeks (FIG. 11). Significant suppression of Varroa population was observed following treatment with the specific triggers (SEQ ID NO:91 and 94) already at week 3.

1. A method of selectively treating an arthropod species for parasites, comprising delivering an effective amount of a nucleic acid molecule, to said arthropod species, wherein said nucleic acid molecule is a dsRNA comprising a sequence selected from SEQ ID NOs: 90-92, and wherein the nucleic acid molecule does not comprise a sequence of 21 or more contiguous nucleotides that are 100% identical or complementary to a gene sequence of a Apis mellifera and Homo Sapiens.

2. The method of claim 1, wherein said treatment decreases the parasitic load of said arthropod species, reduces the death of said arthropod species, or prevents parasitation of said arthropod species.

3. The method of claim 2, wherein said arthropod species is selected from the group consisting of Apis mellifera, Apis cerana, Trigona minima, Halictidae, and Bombus spp.

4. The method of claim 1, wherein said parasites are in the class Acari.

5. The method of claim 4, wherein said parasites are Varroa destructor.

6. The method of claim 1, wherein said delivering comprises a method selected from the group consisting of delivery through a feeder, spraying on hive frames, and contact using an intra-hive device impregnated with said composition.

7. The method of claim 1, wherein said dsRNA comprises the sequence of SEQ ID NO: 90.

8. The method of claim 1, wherein said dsRNA comprises the sequence of SEQ ID NO: 91.

9. The method of claim 1, wherein said dsRNA comprises the sequence of SEQ ID NO: 92.


SEQUENCE LISTING (94 sequences)

SEQ ID NO: 1 - DNA, 450 bp, Varroa destructor
  source 1..450: mol_type = other DNA
atggctgatcagctaactgaggaacagatcgccgagttcaaagaggcgtttagcctgtttgacaaggacggagatggcacgatcacgacaaaggagctcggtacggtaatgcgatctctcggccagaaccccactgaggctgaactgcaggacatgatcaacgaggtcgacgccgacggctccggaacgatagatttccctgagttcctcacaatgatggcaagaaagatgaaggacaccgactcggaggaggagatccgagaggcgttccgcgtattcgacaaggatggcaacggtttcatttcggcggccgagctcaggcacgttatgaccaaccttggcgagaagcttacggacgaggaggtagatgagatgattcgggaggcagatattgacggtgatggtcaggtcaactacgaggagttcgtcaccatgatgacgtccaagtaa

SEQ ID NO: 2 - DNA, 450 bp, Varroa destructor
  source 1..450: mol_type = other DNA
atggcggatcagctgaccgaggagcaaatcgccgaattcaaggaggctttcagcctgttcgataaagacggtgatggcacaattacgaccaaggaactagggaccgtcatgcggtccctcggccagaaccctactgaggctgagcttcaagacatgatcaacgaggtcgacgctgacggtaacggcactattgactttccagagtttctcacgatgatggcgcgtaaaatgaaggacaccgactccgaggaggagatccgggaagcttttagggtttttgataaagacggaaatggcttcatttcggctgcagagctgaggcacgtaatgaccaaccttggcgaaaagctcacggacgaggaagtggacgagatgatccgcgaggcggatatcgacggcgacggacaggtcaactacgaggagttcgtcacgatgatgacatcaaaatga

SEQ ID NO: 3 - RNA, 373 nt, Varroa destructor
  source 1..373: mol_type = other RNA
acagatcgccgagttcaaagaggcgtttagcctgtttgacaaggacggagatggcacgatcacgacaaaggagctcggtacggtaatgcgatctctcggccagaaccccactgaggctgaactgcaggacatgatcaacgaggtcgacgccgacggctccggaacgatagatttccctgagttcctcacaatgatggcaagaaagatgaaggacaccgactcggaggaggagatccgagaggcgttccgcgtattcgacaaggatggcaacggtttcatttcggcggccgagctcaggcacgttatgaccaaccttggcgagaagcttacggacgaggaggtagatgagatgattcgggaggcagatattgac

SEQ ID NO: 4 - RNA, 186 nt, Varroa destructor
  source 1..186: mol_type = other RNA
acaatgatggcaagaaagatgaaggacaccgactcggaggaggagatccgagaggcgttccgcgtattcgacaaggatggcaacggtttcatttcggcggccgagctcaggcacgttatgaccaaccttggcgagaagcttacggacgaggaggtagatgagatgattcgggaggcagatattgac

SEQ ID NO: 5 - RNA, 274 nt, unidentified
  source 1..274: mol_type = other RNA
atacttactggtgctaatttttatcgaggatgcccaactccccccactttaaaactgcgatcatactaacgaactcccgaaggagtgaaaggtgtctatgttgagcttaataacctaccttgcgagcaaagaaggactagttgaccctgggcaccctatattgttatgttgtttcgaactgagttggcacccatgctgcacatgcaacaaacatgtcggccttcgtgtctatcctagaaaagtacctgtgaacttggctgtctacatcatcatc

SEQ ID NO: 6 - DNA, 432 bp, Varroa destructor
  source 1..432: mol_type = other DNA
ctgccggaggaacaggtggctgaatttaaagaggcctttcttttgtttgacaaggacgccgatggaatgattacggccgccgaactaggcgtcgtcatgcgatcgcttggccagcgacctacggagcaagagctcaagaaaatggttaccatggttgaccaggacggcaatggtacaatcgagttcaacgagtttttgatgatgatgtctcgcaagatgaaggaggcagactcggaggaagaactccgggaggcgttccgtgtgttcgatcgagacggtgacggattcatctcgcgggacgagctcagtgtcgtcatgaacaacctcggcgagaaattaagtgacgatgatgttgaggatatgattcgagaggccgatctggacggcgatggcaagattaactaccaagagtttgtgctcattatcacctcc

SEQ ID NO: 7 - DNA, 480 bp, Ixodes scapularis
  source 1..480: mol_type = other DNA
atggctgatcagcttacagaagaacagattgcagttcaaggaggcgttcttcgctgttcgacaaggacggaggatggcaccatcacgaccaaggagctgggcacggtcatgcgctcgctcggccagaacccgacggaggcggagctgcaggacatgatcaacgaggtggacgcagacggcaacggaacgatcgacttccccgagttccttacgatgatggcgcgcaagatgaaggacacggactctgaggaggagatccgggaggcgttccgggtgttcgacaaggacggcaacggcttcatctctgcggcggagctgcgccacgtcatgaccaacctgggcgagaagctgacggacgaggaggtggacgagatgatccgggaggcggacatcgacggggacgggcaggtcaactacgaaggtgggcacgctttccctcccttggttatcctcctgctatgctttctgcagttgctgtga

SEQ ID NO: 8 - DNA, 450 bp, Aedes aegypti
  source 1..450: mol_type = other DNA
atggccgatcaacttacagaagagcagattgccgaattcaaagaagcgttttcgctgttcgacaaagacggtgacggcacaatcacaaccaaggaactgggaaccgtgatgcgatcgttaggccagaaccccacagaagcagaactgcaagatatgataaacgaagtcgacgcggacggcaacggcacgatcgatttccccgaattcctgaccatgatggctcgcaaaatgaaggacaccgatagcgaagaggaaatccgggaggcgttccgagtcttcgacaaggacggcaacggcttcatctcggcagctgagctgcgtcatgtcatgaccaatctcggcgagaagctaacggacgaggaggtggatgagatgatccgcgaagccgacatagatggcgatggccaagttaattatgaagaattcgtaacaatgatgacatcgaagtga

SEQ ID NO: 9 - DNA, 450 bp, Culex quinquefasciatus
  source 1..450: mol_type = other DNA
atggccgatcaacttacagaggaacagatcgccgagttcaaagaagcgttctcgctgttcgacaaagacggtgacggcacgatcacgaccaaggagctgggcaccgtgatgcgatcgttaggccagaaccccacagaagcagagctgcaagacatgataaacgaggtcgatgcggacggcaacggcacgatcgacttccccgagtttctcaccatgatggctcgcaaaatgaaggacaccgatagcgaagaggaaatccgggaggcgttccgagtcttcgacaaggacggcaacggcttcatctcggcggccgagctgcgccacgtcatgaccaatctcggcgagaagctcacggacgaggaggtggatgagatgatccgcgaggccgacattgacggcgatggccaagttaattatgaagaattcgtaacaatgatgacatcgaagtga

SEQ ID NO: 10 - DNA, 450 bp, Acyrthosiphon pisum
  source 1..450: mol_type = other DNA
atggctgatcaactaacagaagaacagattgccgaattcaaagaggcgttttcgctattcgacaaggacggagatggtaccatcaccaccaaagaacttggaaccgtcatgaggtctttaggccaaaatccgactgaagctgaactccaagatatgattaacgaggtcgatgctgatggcaacggcacgatagatttcccagagttcttgactatgatggcccgcaaaatgaaggataccgatagtgaggaagaaatcagagaggctttccgtgtatttgataaggatggaaacggctttattagtgcagctgagctgcgtcatgtgatgactaaccttggagaaaagctcaccgatgaagaggttgatgaaatgatcagggaagctgacattgatggtgatggtcaagtcaactatgaagagttcgtgaccatgatgacttctaagtga

SEQ ID NO: 11 - DNA, 441 bp, Harpegnathos saltator
  source 1..441: mol_type = other DNA
caactcacagaggaacagatcgcagagtttaaggaagccttctcgctattcgacaaagatggcgatggcactataacgactaaagaattgggtacagtcatgcgatccctgggtcaaaatcccacagaggccgagttacaagacatgattaatgaagtagacgcagatggtaacggtacaatcgactttccggagttcttgaccatgatggcacgcaaaatgaaggatacggacagcgaggaggagatcagggaggccttcagagtgttcgataaggatggaaatggtttcatatccgcagcggaactcagacatgttatgacaaatctgggcgagaaactgaccgatgaggaagtagatgaaatgatacgggaggcagatatcgacggcgatggccaagtgaattatgaagaatttgtgacgatgatgacatcaaagtga

SEQ ID NO: 12 - DNA, 459 bp, Pediculus humanus corporis
  source 1..459: mol_type = other DNA
atggtttctttttttttgcgagctgatcagttgaccgaagaacaaattgccgaattcaaggaagcattttccttattcgacaaagatggcgatggtaccataacaactaaggaattgggtacggttatgagatcactcggtcagaatcccacagaagcagaattacaagatatgattaatgaagtggatgcagatggtaatggtaccatcgattttcccgagttcctcaccatgatggctagaaaaatgaaggatacagacagcgaagaagaaattagagaagcattcagagttttcgataaggatggtaatggttttatatcggcagccgagctaaggcacgtcatgacgaacctgggtgaaaaattaacagacgaagaagtggatgaaatgattcgagaggctgatatcgacggagatggacaagtgaattacgaagaatttgtggaaaacttgtga

SEQ ID NO: 13 - DNA, 459 bp, Anopheles gambiae
  source 1..459: mol_type = other DNA
atggccgatcaacttacggaagaacagatcgctgaattcaaagaagcgttctcgctgttcgacaaggacggcgatggcacaatcaccaccaaggaattaggcaccgtgatgcgatcgctaggccagaaccccacagaagctgaattgcaagacatgatcaacgaagtcgacgcggacggtaacggcacgatcgatttccccgagtttctaacaatgatggctcgtaaaatgaaggacacggacagtgaagaggaaatccgggaggcattccgagtcttcgacaaggacggcaacggttttatctctgcagctgagctgcgccacgtcatgactaatctgggcgagaagctaacagacgaggaggtcgacgagatgatccgtgaagccgatatagatggcgatggccaggttaattatgaaggtaagagttgcctttcgcgcccacaccaagcattgttt

SEQ ID NO: 14 - DNA, 501 bp, Solenopsis invicta
  source 1..501: mol_type = other DNA
atgatcggcacgataacgacgagaaattccattatttcagaattcaaagaggcatttatgcttttcgacaaggacgaagatggcacgattacgatggcggaattaggggttgtcatgcggtctctcggtcaaagaccgtcggagacggaactgcgcgatatggtgaatgaggtagatcaagatggaaatggtaccatcgagtttaacgaatttctgcagatgatgtcgaagaagatgaaaagcgccgacggagaggacgaacttcgcgaggcgttccgagtgttcgataagaacaacgatggcttaatatcttcgaaagagttgcgacacgtaatgacgaatcttggtgaaaagctctctgaggaggaggtcgatgatatgattaaggaggcggatctagatggcgacggaatggtcaactacgaaggtaacattttgttttgcctagatgtttattctataatagatttagaatttattctaagcgatatagatgaattg

SEQ ID NO: 15 - RNA, 364 nt, Ixodes scapularis
  source 1..364: mol_type = other RNA
agttcaaggaggcgttcttcgctgttcgacaaggacggaggatggcaccatcacgaccaaggagctgggcacggtcatgcgctcgctcggccagaacccgacggaggcggagctgcaggacatgatcaacgaggtggacgcagacggcaacggaacgatcgacttccccgagttccttacgatgatggcgcgcaagatgaaggacacggactctgaggaggagatccgggaggcgttccgggtgttcgacaaggacggcaacggcttcatctctgcggcggagctgcgccacgtcatgaccaacctgggcgagaagctgacggacgaggaggtggacgagatgatccgggaggcggacatcgac

SEQ ID NO: 16 - RNA, 372 nt, Aedes aegypti
  source 1..372: mol_type = other RNA
gcagattgccgaattcaaagaagcgttttcgctgttcgacaaagacggtgacggcacaatcacaaccaaggaactgggaaccgtgatgcgatcgttaggccagaaccccacagaagcagaactgcaagatatgataaacgaagtcgacgcggacggcaacggcacgatcgatttccccgaattcctgaccatgatggctcgcaaaatgaaggacaccgatagcgaagaggaaatccgggaggcgttccgagtcttcgacaaggacggcaacggcttcatctcggcagctgagctgcgtcatgtcatgaccaatctcggcgagaagctaacggacgaggaggtggatgagatgatccgcgaagccgacataga

SEQ ID NO: 17 - RNA, 373 nt, Culex quinquefasciatus
  source 1..373: mol_type = other RNA
acagatcgccgagttcaaagaagcgttctcgctgttcgacaaagacggtgacggcacgatcacgaccaaggagctgggcaccgtgatgcgatcgttaggccagaaccccacagaagcagagctgcaagacatgataaacgaggtcgatgcggacggcaacggcacgatcgacttccccgagtttctcaccatgatggctcgcaaaatgaaggacaccgatagcgaagaggaaatccgggaggcgttccgagtcttcgacaaggacggcaacggcttcatctcggcggccgagctgcgccacgtcatgaccaatctcggcgagaagctcacggacgaggaggtggatgagatgatccgcgaggccgacattgac

SEQ ID NO: 18 - RNA, 372 nt, Acyrthosiphon pisum
  source 1..372: mol_type = other RNA
acagattgccgaattcaaagaggcgttttcgctattcgacaaggacggagatggtaccatcaccaccaaagaacttggaaccgtcatgaggtctttaggccaaaatccgactgaagctgaactccaagatatgattaacgaggtcgatgctgatggcaacggcacgatagatttcccagagttcttgactatgatggcccgcaaaatgaaggataccgatagtgaggaagaaatcagagaggctttccgtgtatttgataaggatggaaacggctttattagtgcagctgagctgcgtcatgtgatgactaaccttggagaaaagctcaccgatgaagaggttgatgaaatgatcagggaagctgacattga

SEQ ID NO: 19 - RNA, 373 nt, Harpegnathos saltator
  source 1..373: mol_type = other RNA
acagatcgcagagtttaaggaagccttctcgctattcgacaaagatggcgatggcactataacgactaaagaattgggtacagtcatgcgatccctgggtcaaaatcccacagaggccgagttacaagacatgattaatgaagtagacgcagatggtaacggtacaatcgactttccggagttcttgaccatgatggcacgcaaaatgaaggatacggacagcgaggaggagatcagggaggccttcagagtgttcgataaggatggaaatggtttcatatccgcagcggaactcagacatgttatgacaaatctgggcgagaaactgaccgatgaggaagtagatgaaatgatacgggaggcagatatcgac

SEQ ID NO: 20 - RNA, 373 nt, Pediculus humanus corporis
  source 1..373: mol_type = other RNA
acaaattgccgaattcaaggaagcattttccttattcgacaaagatggcgatggtaccataacaactaaggaattgggtacggttatgagatcactcggtcagaatcccacagaagcagaattacaagatatgattaatgaagtggatgcagatggtaatggtaccatcgattttcccgagttcctcaccatgatggctagaaaaatgaaggatacagacagcgaagaagaaattagagaagcattcagagttttcgataaggatggtaatggttttatatcggcagccgagctaaggcacgtcatgacgaacctgggtgaaaaattaacagacgaagaagtggatgaaatgattcgagaggctgatatcgac

SEQ ID NO: 21 - RNA, 372 nt, Anopheles gambiae
  source 1..372: mol_type = other RNA
acagatcgctgaattcaaagaagcgttctcgctgttcgacaaggacggcgatggcacaatcaccaccaaggaattaggcaccgtgatgcgatcgctaggccagaaccccacagaagctgaattgcaagacatgatcaacgaagtcgacgcggacggtaacggcacgatcgatttccccgagtttctaacaatgatggctcgtaaaatgaaggacacggacagtgaagaggaaatccgggaggcattccgagtcttcgacaaggacggcaacggttttatctctgcagctgagctgcgccacgtcatgactaatctgggcgagaagctaacagacgaggaggtcgacgagatgatccgtgaagccgatataga

SEQ ID NO: 22 - RNA, 364 nt, Solenopsis invicta
  source 1..364: mol_type = other RNA
cagaattcaaagaggcatttatgcttttcgacaaggacgaagatggcacgattacgatggcggaattaggggttgtcatgcggtctctcggtcaaagaccgtcggagacggaactgcgcgatatggtgaatgaggtagatcaagatggaaatggtaccatcgagtttaacgaatttctgcagatgatgtcgaagaagatgaaaagcgccgacggagaggacgaacttcgcgaggcgttccgagtgttcgataagaacaacgatggcttaatatcttcgaaagagttgcgacacgtaatgacgaatcttggtgaaaagctctctgaggaggaggtcgatgatatgattaaggaggcggatctaga

SEQ ID NO: 23 - RNA, 373 nt, Varroa destructor
  source 1..373: mol_type = other RNA
acaggtggctgaatttaaagaggcctttcttttgtttgacaaggacgccgatggaatgattacggccgccgaactaggcgtcgtcatgcgatcgcttggccagcgacctacggagcaagagctcaagaaaatggttaccatggttgaccaggacggcaatggtacaatcgagttcaacgagtttttgatgatgatgtctcgcaagatgaaggaggcagactcggaggaagaactccgggaggcgttccgtgtgttcgatcgagacggtgacggattcatctcgcgggacgagctcagtgtcgtcatgaacaacctcggcgagaaattaagtgacgatgatgttgaggatatgattcgagaggccgatctggac

SEQ ID NO: 24 - DNA, 627 bp, Tetranychus urticae
  source 1..627: mol_type = other DNA
attcatcagatttaaaattattcaccttgtacctgaattaaaacaatagtattatataagatggctgaccagctaactgaagagcaaattgctgaatttaaggaggccttttcattgtttgataaagatggtgatggtacaattaccaccaaggaattgggaactgttatgagaagtctaggtcaaaatccaacagaagcggaattacaagatatgatcaatgaagttgatgccgatggtaatggtactattgattttcctgaattcttgactatgatggctagaaaaatgaaggatacagactcagaggaggaaatccgtgaagctttccgtgtttttgataaagatggtaatggttttatttctgctgctgaattgagacatgtaatgaccaatttgggtgaaaaattgaccgacgaagaagtagatgaaatgattcgtgaagccgatattgacggtgatggtcaagttaattatgaagaatttgtaacgatgatgacatccaaatgaataaaagcacaaggttaattgttcattttaattagatccaatggatcccatcagtgacagggataaaaattaatcaataaaagatgaaaagcaaaaaatacatggaagctatcatca

SEQ ID NO: 25 - RNA, 372 nt, Tetranychus urticae
  source 1..372: mol_type = other RNA
caaattgctgaatttaaggaggccttttcattgtttgataaagatggtgatggtacaattaccaccaaggaattgggaactgttatgagaagtctaggtcaaaatccaacagaagcggaattacaagatatgatcaatgaagttgatgccgatggtaatggtactattgattttcctgaattcttgactatgatggctagaaaaatgaaggatacagactcagaggaggaaatccgtgaagctttccgtgtttttgataaagatggtaatggttttatttctgctgctgaattgagacatgtaatgaccaatttgggtgaaaaattgaccgacgaagaagtagatgaaatgattcgtgaagccgatattgac

SEQ ID NO: 26 - DNA, 1114 bp, Varroa destructor
  unsure 1..1114: note = unsure at all n locations
  misc_feature 1100..1110: note = n is a, c, g, or t
  source 1..1114: mol_type = other DNA
ggctcgtacaccgagagagaacggtggactccattgctgtcctgagtgcgtttctcgtcggctgtgtcgtattgcgtgtctgtccgtgctttcgtgcacataagttttctttccacattggagatattgtgaatagtatcctttgtgtttagtgcctgaacaatatttgaaatcgtcgaacgtatcaagacataaaaacgagccaaaatggctgatcaacttacggaagaacaaatcgcagaatttaaggaagcattttcactatttgataaagatggagatggtaccatcacaactaaagagttgggtacagttatgcgatcactaggtcaaaatcccacagaagctgagcttcaggatatgattaatgaagttgatgcagatggtaatggcacaatcgattttccggaattcttaactatgatggctcgtaaaatgaaagatactgatagtgaggaagaaattagggaggccttcagagtatttgataaggatggaaatggtttcatatccgcagcagaactcagacatgttatgacaaatcttggcgagaaactcactgatgaagaagttgatgaaatgattcgggaggctgacattgatggtgatggccaagttaattatgaagaattcgtcacaatgatgacatcaaagtgaatgcaacctgtgtaatggaaaaacttgcaacttggagtggtgttgacgtattaagataaatcaagaaacaaagaaaaatataatgttaacaaaaaacgaatcgaccagaaagtgaaaaaaatcttgtatctggacgcaaagatgtctataaaacgcgaaaaattaacgtccaacacgcgttaatcatcattaacgataagtaatacagggcaattgtttaattaaaagagttataccactaaaaatcattatctctaaatacacaaaacttaattacacaacatgaacataaaaatacatattactcgcgcacatacatgaatacaaaaaaatatacagcacacagaaataccatctacataaaagataatttatttccgtattaaaaagtatataattaaaaaatgttagagatatatatatataatatatatatatatatnnnnnnnnnnngtaa

SEQ ID NO: 27 - RNA, 372 nt, Varroa destructor
  source 1..372: mol_type = other RNA
acaaatcgcagaatttaaggaagcattttcactatttgataaagatggagatggtaccatcacaactaaagagttgggtacagttatgcgatcactaggtcaaaatcccacagaagctgagcttcaggatatgattaatgaagttgatgcagatggtaatggcacaatcgattttccggaattcttaactatgatggctcgtaaaatgaaagatactgatagtgaggaagaaattagggaggccttcagagtatttgataaggatggaaatggtttcatatccgcagcagaactcagacatgttatgacaaatcttggcgagaaactcactgatgaagaagttgatgaaatgattcgggaggctgacattga

SEQ ID NO: 28 - DNA, 822 bp, Varroa destructor
  source 1..822: mol_type = other DNA
ttttttttttttttcgaagaaatcgtatcatcgaacatcggatcgaatttcgatccacgacgctcgaattacatgcaacgaagtgaatgtcagaggggggggggcaggggaaaagtatgcgacgaacgtgtacgtccgtcgtccgctggctcgtcgttaattcgttctatctaacacagacacgagaataagtaagatcctagtagcccggggacagcacctcctcctcctcgtcctcctcgtcgtcgtcgatccccggctctccgagcgcgtggacgaattcgtagaaatcgatacggccgtctccgtccacgtccacttccttgatcatatcctcgatctcttcttccgacaagtcctcgccgagacattgcagcactgccctcaaatcggatgcggtgatgtatcctcgattgtgtttatcgaatacccggaacgcatccctcagctcctgctcttcctgatcctgatccgtgggggcggtttcattcgcacctatgttgctcacgatttccacgaactcttcgaagctgacatttccatccccgtcgatgtcgatctcctgcaacatggtgcgcagctcctcggccctcgcgaattgccccaacgagcgcatcaccctcccgagctcctccttcgtgatgctcccgtccccgtccttgtcgaacagtctgaacgcttccctgaattctttcatttgagatttggatatattgctcggtatcttggtggacgactcagaggcggattttttcggcgacgcaggtaaggagaagaggacgttggtcgacaatttgccggcctccgtggacgaggaggccg

SEQ ID NO: 29 - RNA, 300 nt, Varroa destructor
  source 1..300: mol_type = other RNA
aacgtgtacgtccgtcgtccgctggctcgtcgttaattcgttctatctaacacagacacgagaataagtaagatcctagtagcccggggacagcacctcctcctcctcgtcctcctcgtcgtcgtcgatccccggctctccgagcgcgtggacgaattcgtagaaatcgatacggccgtctccgtccacgtccacttccttgatcatatcctcgatctcttcttccgacaagtcctcgccgagacattgcagcactgccctcaaatcggatgcggtgatgtatcctcgattgtgtttatc

SEQ ID NO: 30 - DNA, 2577 bp, Varroa destructor
  source 1..2577: mol_type = other DNA
ctgctgctgctgctgctgctgctgctgccgtgttaagaactagacaaaggcaacaaccggaagacatctgttaggccagttgggtgaagggaaataaactcgcaacaaagagctactgttgcaaaagctatctgttctgagtcctagactagactgggatcgactaacaactctgcaggccgtggaccactatcactgaacaacgccgcaggaccgagcgattgagaagttggccgcggctgggacgctctggctcacttgatctgattactacttacaccaggtcaatttaaccaagccgttgaaccactcctacatggaaacacacatctacactctcatccaattgtgtacacgcgaagcaaacgacaacggtattagcagcgacactaacaaaaacgaatctgccagaaaccgagtaacgctgatttctgcagcggcgttcctacgaacttaccacagacccggtcggcacaatagtttgaaggttagcatcggcccgtgaactatagtgaaaggaactgagcgaaatactatagttgttgaacagcaggcttcaaagtaaagaagtgaatattcttatggtgacaacaaattttgtctgtagtggtcaagaccctactcaatgaagtgagaagcgaattcatatgtgctcagttatagcagttgtttgtcgtaatgccgccaatatacgacgtctgtgtgtctcgatactggcgtatctgactgattccgatcggtgtcattgatctgcaaagccaattgttttctttacgtcccggatagagcaaacgatcatggcaacggaaacatttggcctgccggaggaacaggtggctgaatttaaagaggcctttcttttgtttgacaaggacgccgatggaatgattacggccgccgaactaggcgtcgtcatgcgatcgcttggccagcgacctacggagcaagagctcaagaaaatggttaccatggttgaccaggacggcaatggtacaatcgagttcaacgagtttttgatgatgatgtctcgcaagatgaaggaggcagactcggaggaagaactccgggaggcgttccgtgtgttcgatcgagacggtgacggattcatctcgcgggacgagctcagtgtcgtcatgaacaacctcggcgagaaattaagtgacgatgatgttgaggatatgattcgagaggccgatctggacggcgatggcaagattaactaccaagagtttgtgctcattatcacctccgccaagtaggccttggagttgctcgcgcacacacatgctattcctcttgtcctacacgacaaacactatacacgttacaatacggaaaaatgaaataaaagcacagtcacacttattcattcattcacagaggatcactggcgtgtcctcattaatatggctgacaaatactattgatttgtctcgactgagctatcaggcacacgcatattgtctgttccgatacgtgcaataataggtaaagtggtgttagcttgagcaccttacggtttacatttattccgtattaacggtactatgtctcaatagtcatgtcgcagcattagccagctaacattaaatttagttgatttttattttatttttttacacaacaagaattatcttactatttggcaagctgactgcgagtagtaaaattaccctagtaaaaaaaaaaggctttaaaaacgatttaacaaaggtgcgtccatttaaaaaagtatgacacaagctaatgtgttcgatcgcggtcggttgcattcggccagtttgtgagacgcgaaatttaccagcagccgtcaacaacgatggaataattatatattaaatataaataataaaatatctgttacacattctatgtgaataaagttatacatatatatatacatacaacagcaatgatatatatgatgatacgtatactatatagaagtatccactgatataggataatgggaaattctggaaaggagaaaacatattcttctttcataattaatgaattatcatcgagcacctcaagaatttgcttagacatattacaaaaaaaaaattaattcactatatataaataatcgcaattaatcaaaaaaaaaatacggtcatttatggcataccctaggttaataaacttcataaaggtacctatatatatatcttttttactgtctctttaatcgtcattatacatagtgacgattaaagagagagagtaaaaaagaggtcaaaataaaagacttgaagtcgattgaggttaatttttattctaggttatatgtgttttagcggatttatagtgtgctcgttgtaaaagttgacgtaaggagttgaaacgcggagagcagtataacagtaaaatggagaggaagtttagaagttgggaaataaatgatttgctactaattggaaattacaaaaatgaccatttaaagaacttatcattagaattttaatatctgccacactgaaccgttttgtcattatttcgaccatagcgattttcacacagttgtctcacgtttgaggccttatcgtttaaaggctaacggaaatttctctaaag

SEQ ID NO: 31 - RNA, 373 nt, Varroa destructor; identical to SEQ ID NO: 23
  source 1..373: mol_type = other RNA

SEQ ID NO: 32 - DNA, 693 bp, Varroa destructor
  source 1..693: mol_type = other DNA
cgcctctatacagtgaaaaagtcggataaggttatgttactcagtaacttaaccttatcttggataacgttatttgacacgtaaacgaaaagtaataacaaagttttgtattatggctcgttattttcgagaggaagatatagatgaattcagggaatgtttttatctatttgcaaggaatggtcaaatacgtactttggatgagcttacaatcattatgagatcattaggattaagtccaactattgcagaattaaataaatatttgaaagataaaggtggaaaaatgtcttttgccgatttcttggaagttatgcatctacaaactagagctgaagatttaccaaaagaagtgatagatgcttttcaagctgcagataaatttaggactggcactataccagctagacagttagcgcatatgttactccactggggtgaacaattaagtaacaaagaagtggagcaaattttcagagaggcaaatgtgtctccaaatggacaagtaaagtacgaagattttgttaaaatagcttgtgcacctgtacctgattactattaaaataaatatttttcatattttttaaagatatttatatactttttacacaatacacacgtatttaattaataaaaggataaaaatgatcataaaagaaaaagaatttattcttccagcaacttatcttcgac

SEQ ID NO: 33 - RNA, 315 nt, Varroa destructor
  source 1..315: mol_type = other RNA
atgcttttcaagctgcagataaatttaggactggcactataccagctagacagttagcgcatatgttactccactggggtgaacaattaagtaacaaagaagtggagcaaattttcagagaggcaaatgtgtctccaaatggacaagtaaagtacgaagattttgttaaaatagcttgtgcacctgtacctgattactattaaaataaatatttttcatattttttaaagatatttatatactttttacacaatacacacgtatttaattaataaaaggataaaaatgatcataaaagaaaaagaatttattctt

SEQ ID NO: 34 - DNA, 612 bp, Varroa destructor
  source 1..612: mol_type = other DNA
cgtttttttaatttcaaagactcctgattttcttttcttttcctttcctacaaaccaaacacaagctagaaagaggttcgatttttgcaggaagaagaaggaacaatggctgatcagctcaccgatgaccagatctctgagtttaaggaagccttcagcctcttcgataaggatggagatggttgtatcaccaccaaggagcttggaactgtgatgaggtctcttggccagaaccccactgaggcagagctccaggacatgatcaacgaggtggatgctgatggcaatggaacaattgactttcctgagttcttaaacctcatggccaggaagatgaaggatactgattctgaggaggagctcaaggaagctttccgcgtgtttgacaaggaccagaatggcttcatttctgcggctgagctccgccatgttatgacgaatcttggtgagaagctcacagacgaggaagttgatgagatgatccgtgaggctgatgtagatggtgacggccagattaactacgaggagtttgtcaaagtcatgatggccaagtgaggatcattaaccaaaccttaaaatttcgaaagcataaacatttaaaaaaaaaaaaaa

SEQ ID NO: 35 - RNA, 371 nt, Varroa destructor
  source 1..371: mol_type = other RNA
cagatctctgagtttaaggaagccttcagcctcttcgataaggatggagatggttgtatcaccaccaaggagcttggaactgtgatgaggtctcttggccagaaccccactgaggcagagctccaggacatgatcaacgaggtggatgctgatggcaatggaacaattgactttcctgagttcttaaacctcatggccaggaagatgaaggatactgattctgaggaggagctcaaggaagctttccgcgtgtttgacaaggaccagaatggcttcatttctgcggctgagctccgccatgttatgacgaatcttggtgagaagctcacagacgaggaagttgatgagatgatccgtgaggctgatgtaga

SEQ ID NO: 36 - DNA, 659 bp, Ixodes scapularis
  source 1..659: mol_type = other DNA
acaccgcgctcgacgagaactcgcgcaaggagcgcgtgctaacgccgatagctctcgtacaaaagaaaactaaagacttgacacgctcaataccgatctttcacattttcagcaaaatttgagcattccgaatatggacctgactccggaagagatcgcggacatcaagggagcgtttctgctgtttgaccgcaacggcgacggaaccatctccacgactgagctagagatggtcctccgcgccatgggcgaacggcccagtccttcccagctggcccgtatagtgcggcaaattgacagcgaccgcaatggaagcatcgacttccaagagtttctctttttcatggccggcaggatttcccacaaaggcctctccaaaagcgcagtcctcaaggccttccaactcttcgaccgcgatggcaatggatacatcaccagggaggaactcgtccacattttcacgcacgttgggcagagcatgagccaagaagacgccgaaaagataatccgcgaagtggatgtggacaaggacggaaagatccattacactgaattggtcaacaaggtgctgcccaccaagaagcaaaaagaagaaaccaaaacctagaaggtcgtcgcttggcacggtctttattattaaacaagtgctttatcgcttg

SEQ ID NO: 37 - RNA, 374 nt, Ixodes scapularis
  source 1..374: mol_type = other RNA
agatcgcggacatcaagggagcgtttctgctgtttgaccgcaacggcgacggaaccatctccacgactgagctagagatggtcctccgcgccatgggcgaacggcccagtccttcccagctggcccgtatagtgcggcaaattgacagcgaccgcaatggaagcatcgacttccaagagtttctctttttcatggccggcaggatttcccacaaaggcctctccaaaagcgcagtcctcaaggccttccaactcttcgaccgcgatggcaatggatacatcaccagggaggaactcgtccacattttcacgcacgttgggcagagcatgagccaagaagacgccgaaaagataatccgcgaagtggatgtggac

SEQ ID NO: 38 - DNA, 549 bp, Ixodes scapularis
  source 1..549: mol_type = other DNA
gattcgtccacttattttgtccctattcttcgtccgcagtcgtcctcgaggaaaagtgcgtcaggtgcgggctacaagcggaaactgagcggaaagccagaaccgagcgaggagcagaagaatgacatgaaggaagcgttcagtctcttcgatcccagtggcacgggcttcatggagtctaaagatatgaagtttgcaatgagagcactgggttttgaaccaaaaaaggaggaagtgaaaaaactgatagcagagattgacaagcaggggactggaaaaattcccttggaggagttcatgagcgtcatgtccacgaggctggctgagaaagacataaatgaggagattatgaaggcgtttcagctgtttgatgaggatggcactgggaagatttcttttaagaacctcaagaatgtggccaaggaactgtcggagaacctcacagatgaggagcttcaggaaatgatcaatgaagctgacagggatggagatggcgaagtgaaccaagaggagttccttaggataatgaagaagacctgcctctactga

SEQ ID NO: 39 - RNA, 373 nt, Ixodes scapularis
  source 1..373: mol_type = other RNA
tgaaggaagcgttcagtctcttcgatcccagtggcacgggcttcatggagtctaaagatatgaagtttgcaatgagagcactgggttttgaaccaaaaaaggaggaagtgaaaaaactgatagcagagattgacaagcaggggactggaaaaattcccttggaggagttcatgagcgtcatgtccacgaggctggctgagaaagacataaatgaggagattatgaaggcgtttcagctgtttgatgaggatggcactgggaagatttcttttaagaacctcaagaatgtggccaaggaactgtcggagaacctcacagatgaggagcttcaggaaatgatcaatgaagctgacagggatggagatggcgaagt

SEQ ID NO: 40 - DNA, 234 bp, Ixodes scapularis
  source 1..234: mol_type = other DNA
atggccaagaacgtccgcgccctggacaccgaggaggagattttggaggccttcaaagtcttcgaccgcaacggcgacggcttcgtgagcacagccgagctccgtcacgtgatgaccacgttaggcgagaagttgacgcacgaagaagtggacgagatgatccgcgaggccgaccgcgacggcgacggacagatcaactacgacgagttcgtggccatgatgacttccaagtga

SEQ ID NO: 41 - DNA, 213 bp, Ixodes scapularis
  source 1..213: mol_type = other DNA
cctggaaccgaggaggagattctggaggccttcaaagtcttcgaccgcaacggcgacggcttcgtgagcacggccgagctccgtcacgtgatgaccacgctaggcgagaagttgacgcacgaagaagtggacgagatgatccgcgaggccgaccgtgacggcgacggacagatcaactacgacgagttcgtggccatgatgacctccaagtga

SEQ ID NO: 42 - RNA, 200 nt, Ixodes scapularis
  source 1..200: mol_type = other RNA
gaggaggagattctggaggccttcaaagtcttcgaccgcaacggcgacggcttcgtgagcacggccgagctccgtcacgtgatgaccacgctaggcgagaagttgacgcacgaagaagtggacgagatgatccgcgaggccgaccgtgacggcgacggacagatcaactacgacgagttcgtggccatgatgacctccaa

SEQ ID NO: 43 - DNA, 450 bp, Aedes aegypti
  source 1..450: mol_type = other DNA
atgtccgcccatagtctaacagaggaacaagggcgccagttccgtcagatgttcgagatgttcgacaaaaatggcgacggttcgatcagcacatcggaactgggatcggtcattcgggccttgggtatgaatccctccattgcggaaatcgagcaaatgatccacgaggtcgatttggacggaagtgggtcgattgagttgaacgaatttctcatactgatggcacgtaagtcacgggagggttccacacaggaagagctacgggatgcgttcaaaatttttgacaaggatggagatggatttctcacggttgacgagttgtcggctgttatgaagaactttggcgagagattgaccgatgacgaactagcagatctgctggaggaagccgacatcgatggagacggaaagatcaactatgaagaatttgtcatcatgttgagcaagtga

SEQ ID NO: 44 - RNA, 300 nt, Aedes aegypti
  source 1..300: mol_type = other RNA
acagaggaacaagggcgccagttccgtcagatgttcgagatgttcgacaaaaatggcgacggttcgatcagcacatcggaactgggatcggtcattcgggccttgggtatgaatccctccattgcggaaatcgagcaaatgatccacgaggtcgatttggacggaagtgggtcgattgagttgaacgaatttctcatactgatggcacgtaagtcacgggagggttccacacaggaagagctacgggatgcgttcaaaatttttgacaaggatggagatggatttctcacggttgacgag

SEQ ID NO: 45 - DNA, 465 bp, Aedes aegypti
  source 1..465: mol_type = other DNA
atgtccgcccaaaccccgccagacaagctttcccaggatcaaatcgaagaactgcgggaagctttctccctgttcgacaccaacggcgacggaaccataacctgttcagaacttggcacagtccttcgatcccttggcaaaaatgtatccgacgcggaagtggaagaactgctcaaagaagtcaacgtcgaccacgaaggaatgatccactttccggacttcgtggcaatgatgtccatccgattgcgggacttcaatagcgaggaggaactcaaggaagccttccggatcttcgaccgcaacggagatgggctgatttcggcggacgaattgcgagcggctctccaatctttcggggaacagctggccgaggaggaaatcgaagaactgctccgggaggcggatgtcaactgcgacggacaaatagactacgaggagtttgttaaaatgatcacgctgaaataa

SEQ ID NO: 46 - RNA, 300 nt, Aedes aegypti
  source 1..300: mol_type = other RNA
caaatcgaagaactgcgggaagctttctccctgttcgacaccaacggcgacggaaccataacctgttcagaacttggcacagtccttcgatcccttggcaaaaatgtatccgacgcggaagtggaagaactgctcaaagaagtcaacgtcgaccacgaaggaatgatccactttccggacttcgtggcaatgatgtccatccgattgcgggacttcaatagcgaggaggaactcaaggaagccttccggatcttcgaccgcaacggagatgggctgatttcggcggacgaattgcgagcg

SEQ ID NO: 47 - DNA, 1150 bp, Aedes aegypti
  source 1..1150: mol_type = other DNA
tcgtcaaggatatccggcaactgattatgacccggcaaaccaacgatcccaacggccagcaacaggaacagaacgaaccagaatccagtcagaacacccagagcgaccaaagcaacaaccagcccacgcagcgattgggaggaaccacctcggacttcagtgtcagctccgcggccactaatcgaagtatgccccgccaccaggaggacaatcccaatcaaccggccagcgactgttccagcctcgagggaaatgtattcgtcgaaggcggatccggcaccggagcgcacccgaaaacacgccgctcgcaaacttccgattcgatcacctccagcaacttcaactacagtctcaaccggaggttcatatcgaagaaccagatgaaggagtttcgagaagcgttccggctgttcgacaaggataatgacggctcaatcaccaaggaagaactgggaactgtcatgaggtcgttgggacaatttgctcgcgtggaagaattacaagagatgttactggagattgatgttgatggcgatggaaacgtaagtttcgaagagtttgtcgacatcatgtccaacatgacggataccgtggcggaaacatcggccgaccaggaggaacgtgagctacgtgatgccttccgtgtcttcgacaagcacaatcgaggttacattacggcatcagatctacgggcggttcttcaatgtctgggcgaagatttggatgaagaagaaattgaagacatgatcaaagaagtggacgtggatggagacggacggatcgatttctacgaattcgtacatgctcttggagaaccggaagattcccaagaaaacgacgacgaagacgaggcagtgtccccccattcgctgtcctgtgacgtgcatgtctaagaaccgccaggagaaaaatagctaacgccaacgaatcgcattcctaacaaaatgtcgaacaatctagagacattgaccagattttttttaaatatttaacacacaaaaaaacttcgcttaacgccattgtacttctccatacgcttgataacagattccagaacacctaatgaatttatcaatctatacataataactattcatctctaatcacgaaaaaagtttaaataaacatatcaaattgagcaaccaataag

SEQ ID NO: 48 - RNA, 300 nt, Aedes aegypti
  source 1..300: mol_type = other RNA
gagtttcgagaagcgttccggctgttcgacaaggataatgacggctcaatcaccaaggaagaactgggaactgtcatgaggtcgttgggacaatttgctcgcgtggaagaattacaagagatgttactggagattgatgttgatggcgatggaaacgtaagtttcgaagagtttgtcgacatcatgtccaacatgacggataccgtggcggaaacatcggccgaccaggaggaacgtgagctacgtgatgccttccgtgtcttcgacaagcacaatcgaggttacattacggcatcagat

SEQ ID NO: 49 - DNA, 450 bp, Culex quinquefasciatus
  source 1..450: mol_type = other DNA
atgtcggcccactcgctcaccgacgaacagcagcgccagtaccggcaaatgttcgaaacgttcgacaaggatggcaacggttccatcacgacgacggaactgggaacgctagtgcgagcgctaggtcttaatccttcgatcgccgagatcgagcagatgatccacgaggtcgacctggacggaagcgggacgatcgagctgaacgagttttacgtgctgatggcccggaagcatcgggaagcctcgtcggaggacgagctgaggcaggctttcaaggtgtttgacaagaacgaggatgggttcttgacggtggaggaactgtcgatggtgatgaagaactttggtgagcggttgagcgatgaagagttggcggatttgttggaggaggcggatgttgacaaggacggtcggattaattacgaggaatttgtgaccatgttgaccaagtag

SEQ ID NO: 50 - RNA, 300 nt, Culex quinquefasciatus
  source 1..300: mol_type = other RNA
acagcagcgccagtaccggcaaatgttcgaaacgttcgacaaggatggcaacggttccatcacgacgacggaactgggaacgctagtgcgagcgctaggtcttaatccttcgatcgccgagatcgagcagatgatccacgaggtcgacctggacggaagcgggacgatcgagctgaacgagttttacgtgctgatggcccggaagcatcgggaagcctcgtcggaggacgagctgaggcaggctttcaaggtgtttgacaagaacgaggatgggttcttgacggtggaggaactgtcgat

SEQ ID NO: 51 - DNA, 477 bp, Culex quinquefasciatus
  source 1..477: mol_type = other DNA
atgtcatcgtcttcccgccaccccaaacccaccgcggaccccctgaccaaggagcaaatcgaagaactgcgcgaagcgttcaccctgttcgacaccaacggcgacggaacgatttccggctcggaactgtccaccgtgctgcgggccctcggcaagaacgtctcggacgccgaagtcgaggaactgctgaaggaggtccgcaccgacgacgagggccgcatccggttcggggactttgtggccatgatgacggtccggttgaaggactttaacaacgaggaccagctgcaggaggcgtttcggatcttcgatcgggacgggaatgggcggatttcggcggaagagctacgggtcgcgttgaggtcgtttggggagcagttgaccgaagaggagctggaggagttgctgcgcgaggcggacgtcaacagtgacggccagattgactacggggagtttgtgcggatgataacgcagtga

SEQ ID NO: 52 - RNA, 300 nt, Culex quinquefasciatus
  source 1..300: mol_type = other RNA
gcgaagcgttcaccctgttcgacaccaacggcgacggaacgatttccggctcggaactgtccaccgtgctgcgggccctcggcaagaacgtctcggacgccgaagtcgaggaactgctgaaggaggtccgcaccgacgacgagggccgcatccggttcggggactttgtggccatgatgacggtccggttgaaggactttaacaacgaggaccagctgcaggaggcgtttcggatcttcgatcgggacgggaatgggcggatttcggcggaagagctacgggtcgcgttgaggtcgtttg

SEQ ID NO: 53 - DNA, 927 bp, Culex quinquefasciatus
  source 1..927: mol_type = other DNA
atgaccgattttctccagcttccccaatgcaatactcgacaaaccaacgacccaaacagcgccgagcagcaacaacagagcgaacccgaatcgaaccagagcagcacgcaccagagcagcagccacagctaccagcagccgtcgtcaacgcagcaccggttggccggatctccgtcgagcagtgccaacgcgagtcccgtgatcggccggaacatgccccgccaccagcaccaggacaccgcgcccagtgacgatgggacctcgagcagtctggacgggagtgtctttgccgccgacggaactccgaccgctccgggggcgttggccaggacgcgccgctcgcagacctcggaatcgatcacctccagcaacttcaactacagtttgaaccggaggttcatctccaagaaccagatgaaagagttccgggaggcgttccggctgtttgacaaggacaacgacgggtcgatcacgaaggaggagctgggcacggtgatgcgatcactggggcagtttgcccgtgtcgaggaactgcaggagatgctgctggagattgacgtcgatggtgatggcaacgtcagcttcgaggagtttgtcgacatcatgtccaacatgaccgacacggtggcggaggcatccgccgaccaggaggagcgcgaactccgggatgcgttccgcgtgtttgacaagcacaaccggggctacatcacggcgtctgatctgcgggcggttctgcagtgtctgggagaagatttggacgaggaagaaatcgaagacatgatcaaggaggtggacgtcgacggcgatggacggatcgacttttacgagtttgtgcacgccctcggagagccggaagattcacaggagaacgacgacgaggaggaccccctgtcacctccgtcactgtcgtgtgacgtaaacgcctaa

SEQ ID NO: 54 - RNA, 300 nt, Culex quinquefasciatus
  source 1..300: mol_type = other RNA
gagttccgggaggcgttccggctgtttgacaaggacaacgacgggtcgatcacgaaggaggagctgggcacggtgatgcgatcactggggcagtttgcccgtgtcgaggaactgcaggagatgctgctggagattgacgtcgatggtgatggcaacgtcagcttcgaggagtttgtcgacatcatgtccaacatgaccgacacggtggcggaggcatccgccgaccaggaggagcgcgaactccgggatgcgttccgcgtgtttgacaagcacaaccggggctacatcacggcgtctgat

SEQ ID NO: 55 - DNA, 333 bp, Culex quinquefasciatus
  source 1..333: mol_type = other DNA
atggtcggtgtccaagctggccatttgatatctcaatcacggcggggcggctccgcgtgcgaaaacggggaaattttgattgatgacggcggcggcggggagcggcgggttttaaacctgttctacaaggggaataaaaatgccgatcaacttacagaggaacagatcgccgagttcaaagaagcgttctcgctgttcgacaaagacggtgacggcacgatcacgaccaaggagctgggcaccgtgatgcgatcgttaggccagaaccccacagaagcagagctgcaagacatgataaacgaggtcgatgcggacggactgcatccgctttaa

SEQ ID NO: 56 - RNA, 300 nt, Culex quinquefasciatus
  source 1..300: mol_type = other RNA
gtcggtgtccaagctggccatttgatatctcaatcacggcggggcggctccgcgtgcgaaaacggggaaattttgattgatgacggcggcggcggggagcggcgggttttaaacctgttctacaaggggaataaaaatgccgatcaacttacagaggaacagatcgccgagttcaaagaagcgttctcgctgttcgacaaagacggtgacggcacgatcacgaccaaggagctgggcaccgtgatgcgatcgttaggccagaaccccacagaagcagagctgcaagacatgataaacgag

SEQ ID NO: 57 - DNA, 934 bp, Acyrthosiphon pisum
  source 1..934: mol_type = other DNA
gctgtgcacgttttgtttacccgaaaaatgtgattcaaactttcagttttttaaatcttaactgcgttgtttaagaaaaaaaaaaccctaaatttattattgtttattaataataaatcaatcgatcgaataatcgtttgcgggtatacctaagtacgaagtaaaatatgagtgcacaaaatagtgataatgaccgttataaaaaggaatattcgagaataaggaaactgacgagtagatatgacatacggactcaatcaaatgaatttggtttgtcggaagatcaagttgcggaattcaaagaagcatttatgttgttcgataaagaccatgatggacggattactgaggcagaactaggagtggtcatgagatctttgggtcaaaggcctactgaaactgatttgcgaggtatggttaaagaagtggataaagatggcaatggtagtattgagtttgatgaattcctgctaatgatggctagaaaactaaaagcagcagatggcgaggaagaaatgcaccaagcttttaaagtatttgacaaaaatggcgatggattcataacatttgatgaactcaaacgtgttatgtgcagtatcggagaaaggctcactgatgaagaaattgaggacatgataaaagaagcagatttaaatggtgataaaaaaattgattataaagaatttattacaataataagttctaagaaataaaacgaattacggacttggatgtaccctcatatggcattcgttcctcatctgcatctgtgtcattggctgctaagactttacttaataataaaccttgatcttctctactagaataaaatagctctggatcctaaagtaaaatatacagtataattttaaaatagtcgtatacaaatttttttttaaatattgagtgtacctcatccattccagcaagtttc

SEQ ID NO: 58 - RNA, 372 nt, Acyrthosiphon pisum
  source 1..372: mol_type = other RNA
tcaagttgcggaattcaaagaagcatttatgttgttcgataaagaccatgatggacggattactgaggcagaactaggagtggtcatgagatctttgggtcaaaggcctactgaaactgatttgcgaggtatggttaaagaagtggataaagatggcaatggtagtattgagtttgatgaattcctgctaatgatggctagaaaactaaaagcagcagatggcgaggaagaaatgcaccaagcttttaaagtatttgacaaaaatggcgatggattcataacatttgatgaactcaaacgtgttatgtgcagtatcggagaaaggctcactgatgaagaaattgaggacatgataaaagaagcagatttaaa

SEQ ID NO: 59 - DNA, 2509 bp, Acyrthosiphon pisum
  source 1..2509: mol_type = other DNA
gatttttcgtttaaataacccccccgggtttcggataaactcggtgtgcgtgtggacgccgccgccgccgcgtgagcgttatttcgctcgctgtgattacgaacgctcgtgcagtcgtcgtcatgcagcatctccgagacccggcgaaagaccatcagagtacgtgaacactgcacatcacactccatcgcgatatacttatctgtaggacgacaccctttaccgaggacacaatgatatacacgttataatactatcgtacatcgtatatattgtattcttatcatctaatattatattatcacaatcgttatattgtattatagaattattgtatatcgttttaatttgacacgatacgacatttggtgttgagaatcgcgcagagagagaaagagagagagaaaaagagagaatagatattttgatgtaaataattattacaatataatattgtatttgaaaatagaaacagataattggtcgttgaggcagttctcacctttttaagatatacatattatattatatataaacagtattttgttttactcctttgagtgaacatttcgtttaactataccacaatttattaatattatataatatctatcatggacggtaaaatatcatcggtattcgaaaaatacttttcaccttcaccgctggtgaaccaaataaggaattttgtcaatcggcaaatagacgagcaaacgccacaaaacgtaagcacacaaccaaccgctgtagcgaaacttcacagtaataatgtagtgaacggcaccactccgaaacccatcacaagcaacgaaaaaccaacggaagtccaagtacatccacaacccactcagcctgccgatagaaacttagtgcacgttactaaagcacagatgaaagaatttcaagaagcatttaggttatttgataaagacggtgacggcagcatcactaaagaggaattgggtcgagttatgcggagtcttggacagtttgctagagaagaagaattggagacaatgttacaagaagtcgacatcgatggcgatggagcgttcagttttcaagagtttgtagaaattgtgtacaatatgggtggtacagcagaaaagacggcggaccaagaggaaaaagagctccgagacgcatttagggtatttgacaaacataaccgaggatatataagtgcgtcggacttgagagctgtccttcaatgtttgggtgaagatttgtcagaagaagaaatcgaggatatgatcaaggaagtagacgtggacggagatggaagaatcgatttttacgaatttgtgaatgcccttggagaaccaggagatgattatgatgaaaacgacgaagatgaagaagatatttatccccaattggacattcaaacataacttataaacaattacacgttttagtatgatgcgataacaagttcagttaaaattgaattatcaaaaatgataacaatattttttgtaaacttaggttaaaatgttaaaaacttacccattttgtaatttctatcaggaataaacacaccaatatctgttttttttttgccaactaatgatctctaataagtaaaacatattttactttaatatcaatgtactactgtagtattgttaagtaatttatgtcattttcaatgtttaatgtataaattaatcatttaatggtacaatcagtaattcaccttaagacccaattatttattaaaaacatattatatcattaatttacttaatgtatacattttagtcaaaaaacattcagttatatgttataagtcgttataactatttatttaagaaactataaaaaattattatttattataatagtttacgtatctatttacttccatggaaggtatttataaatagaaaatgttaaggtaacttaattaaaacaataattatgaacaagtatttttactttaaaaaaattacataattatattgtcagtattctttcaaatacaattaattcaaatttgtgaaattgtccattcctcctttatgctttgtgaactaaatttaaatacattaagaaccagagtacaattaaaggaaatagtcagctgcgtgtttttttgttggcattcctcggttttcttgtggtgctgcctcaaatattgtaaaatctctttgaagtgtttcactgagttccaatattgccgcaacgttaccacatctattatttacaattagtattaacaattaaataacagcattaataaataaatagtataatgtaatttttagagtagtatttaccgataacaatagttgggtgctgaccaaacagtaagcacggtttcatcaaaatgccatttatatccttccatgactagttgatgagctcgacaaataatatctatatcatttgtagtattaaactgggagacaacatcagaaccaaataggtatccagcaccccgaggactcacaccccatccctgagtatctaaaataattgtgtatgttaaaaatctatttttttt

SEQ ID NO: 60 - RNA, 294 nt, Acyrthosiphon pisum
  source 1..294: mol_type = other RNA
acagatgaaagaatttcaagaagcatttaggttatttgataaagacggtgacggcagcatcactaaagaggaattgggtcgagttatgcggagtcttggacagtttgctagagaagaagaattggagacaatgttacaagaagtcgacatcgatggcgatggagcgttcagttttcaagagtttgtagaaattgtgtacaatatgggtggtacagcagaaaagacggcggaccaagaggaaaaagagctccgagacgcatttagggtatttgacaaacataaccgaggatatat

SEQ ID NO: 61 - DNA, 477 bp, Pediculus humanus
  source 1..477: mol_type = other DNA
atggccgacgaaacgacgacggaattaccggaggaaaatctttcggttgaaaaaatcgcagaattccgcgaagctttcaaccttttcgacaaagatggcgacggtaacataacgaccaaagaattgggtacttgcatgaggtctctcgggcagaatccgacggaagcggaaatcgcggagctgatttgcgaagtagacgtagagggaacaggtttaatcgatttcacatcgttcgttttgataatggctaaaaagataaaagacgtcgacaacgaggaagaactcagagaagcttttagaatattcgataaggaaggtaacggattcataaccgcatccgagctcaggcacataatgatgaacttgggtgaaaaattaacggaagaagaatgcgacgaaatgattagggaagcggatgtcatgggtgacggaaatatcaattacgaagaattcgtcaccatgatgatgtcaaagtga

SEQ ID NO: 62 - RNA, 379 nt, Pediculus humanus
  source 1..379: mol_type = other RNA
aaaaatcgcagaattccgcgaagctttcaaccttttcgacaaagatggcgacggtaacataacgaccaaagaattgggtacttgcatgaggtctctcgggcagaatccgacggaagcggaaatcgcggagctgatttgcgaagtagacgtagagggaacaggtttaatcgatttcacatcgttcgttttgataatggctaaaaagataaaagacgtcgacaacgaggaagaactcagagaagcttttagaatattcgataaggaaggtaacggattcataaccgcatccgagctcaggcacataatgatgaacttgggtgaaaaattaacggaagaagaatgcgacgaaatgattagggaagcggatgtcatgggtgac

SEQ ID NO: 63 - DNA, 537 bp, Pediculus humanus
  source 1..537: mol_type = other DNA
atggatgctaggaatgaagtttacaacaccgaatataatcgtttaagaaaattgacgtgtagaacggaaattaaattatcttgctctgaatatggtcttacggaggaacaagtcgctgaatttaaagaagcttttatgctttttgacaaagatgaagatggacaaataacaatggccgaattaggagtcgttatgagatctttgggacaacgtccgacagaaacggaattaagagacatggttaaagaggttgatcaagatggaaatggtacaatcgaattcaatgaatttttacaaatgatggcaaaaaaaatgaaaggagctgatggtgaagaagaacttcgagaagcattcagggtgtttgataaaaataacgatggactcatttcatccattgaacttcgacatgtcatgacaaatttaggtgagaaactttcagacgaagaagttgatgatatgataaaagaagcagatttagatggagatggtatggttaactacaatgaatttgtaacgatattaacatcaaaaaattaa

SEQ ID NO: 64 - RNA, 417 nt, Pediculus humanus
  source 1..417: mol_type = other RNA
acaagtcgctgaatttaaagaagcttttatgctttttgacaaagatgaagatggacaaataacaatggccgaattaggagtcgttatgagatctttgggacaacgtccgacagaaacggaattaagagacatggttaaagaggttgatcaagatggaaatggtacaatcgaattcaatgaatttttacaaatgatggcaaaaaaaatgaaaggagctgatggtgaagaagaacttcgagaagcattcagggtgtttgataaaaataacgatggactcatttcatccattgaacttcgacatgtcatgacaaatttaggtgagaaactttcagacgaagaagttgatgatatgataaaagaagcagatttagatggagatggtatggttaactacaatgaatttgtaacgatattaac

SEQ ID NO: 65 - DNA, 648 bp, Pediculus humanus
  source 1..648: mol_type = other DNA
atgactacgaaacataatatatctggaagaatacgggacgaaccagggggacaaagggaaaaaaatggaaatgtaaccggtcgaacaataacatatccaacaacagggaacaaaagaaatattgatacaatgacgaaaaataacatatcaaaatcgcaaatgaaggaatttcgagaagcttttcgactttttgacaaagatggtgatggtagtataactcaagaagaacttggaagagttatgagatctttaggacaatttgccagagaagaagaactacaagaaatgcttaaggaagttgatatagatggagatggaaattttagctttgaagaatttgttgaaatcgtatcaaatatgggaggtgcagcaactgaaaaaacagctgatgaagaagagaaagaacttagagatgcttttagagtatttgataaacataatcgaggttttataagtgcttctgatcttcgagctgttttgcaatgtctgggtgaagaattatcagaagaagaaaaaatgataagagaagttgatgtggatggagatggtagaattgattttttcgaatttgttcgagctttgggtacacactacaggcaaaatttctttttcaggtttctatccatttatattattagcacagtttga

SEQ ID NO: 66 - RNA, 449 nt, Pediculus humanus
  source 1..449: mol_type = other RNA
atcgcaaatgaaggaatttcgagaagcttttcgactttttgacaaagatggtgatggtagtataactcaagaagaacttggaagagttatgagatctttaggacaatttgccagagaagaagaactacaagaaatgcttaaggaagttgatatagatggagatggaaattttagctttgaagaatttgttgaaatcgtatcaaatatgggaggtgcagcaactgaaaaaacagctgatgaagaagagaaagaacttagagatgcttttagagtatttgataaacataatcgaggttttataagtgcttctgatcttcgagctgttttgcaatgtctgggtgaagaattatcagaagaagaaaaaatgataagagaagttgatgtggatggagatggtagaattgattttttcgaatttgttcgagctttgggtacacactacaggcaaa

SEQ ID NO: 67 - DNA, 648 bp, Pediculus humanus
  source 1..648: mol_type = other DNA
atgatgaaaaacaaaactgacagcagtctcggagctgagcattcagatttgaaacattcgacgagtgaaaccgaagaactccaatcctcggaactcgaagtgataaaagatgaagagcctcaaatagatctgagacagtttctgacgaaagaacaagtgcaagaattcaaaagaattttccaggcttacgatgtcaacaacgaagataaaatcccggtcaaagccatcggaatcattttgagaaacatgggattgaatccgtccaaggcgattctcaagagaatgacaaaggaaatcgatccggataaaaacggttacgtggatttcgaaatgtttttacatcccatggcacgaatgatacacgaagtcccggaaaatcacgaggacataatcgcagcattcaaagttttcgacgaagacgacgaaggtttcgtatccgttaaagctttgaccgaatacctcacgaacctgggcgaagatttggaagatttcgaaattgataatttgattaaaatggcggatcccaaaggcacgggccgagtctactacgaaggattcgtcgagaaaattttcggaatcgtaagaaacgggaaaaaaaagaaaaatctcaaagggaaaaagggaaaaaaacgaaaaaaaaatgaatga

SEQ ID NO: 68 - RNA, 451 nt, Pediculus humanus
  source 1..451: mol_type = other RNA
tcaaatagatctgagacagtttctgacgaaagaacaagtgcaagaattcaaaagaattttccaggcttacgatgtcaacaacgaagataaaatcccggtcaaagccatcggaatcattttgagaaacatgggattgaatccgtccaaggcgattctcaagagaatgacaaaggaaatcgatccggataaaaacggttacgtggatttcgaaatgtttttacatcccatggcacgaatgatacacgaagtcccggaaaatcacgaggacataatcgcagcattcaaagttttcgacgaagacgacgaaggtttcgtatccgttaaagctttgaccgaatacctcacgaacctgggcgaagatttggaagatttcgaaattgataatttgattaaaatggcggatcccaaaggcacgggccgagtctactacgaaggattcgtcgagaaaatt

SEQ ID NO: 69 - RNA, 1546 nt, Varroa destructor
  source 1..1546: mol_type = other RNA
gtttagcccattttcgcgctgtgtctgtcggcgactgcggaagtactgagctagctgagtcgtggtgcttagaaggcagtggcagtgagtggcatcagcggtaataagtgagggacaacagccggagagtcgtgtcggttggtaggtcggttcgttgccgattaatgccccacatgtgagttacgtgcggtgcttgttccgctgtgcattggacgttacatacagagagtcaggtgtagtttattttagacgaaaaactaccagcactacctgatacagcgacccaacgtagagagggaaagagaaagagctgttgtttcgctaggttagttctgaacaattggattgattcgcaaatgtacgcttgtacggctaacggttgaacggactgtgcaaactgcggatagtgcgtgaactagcagacaacatatccaataactaactacacggttattattgatacaaacacctagttcagacagacaatttcggtttgctttgttggcactataaggagtaagtgatattgttttttgtttatcagtcggcctagtattgtcggtgcacttacggataacaggagaggcagtacaaaggcaactcgattcactatcgttcacgggcgctaatacagcgaaatattaataggcaaagcaagcaagccagccggccaagcccaaaccccgggcgaaacgcagataccaacagcgctgaagtgcgtggcaaacgacaacgggacagtagggcataagctagacagcatatagcttttctaaccatggctgatcagctaactgaggaacagatcgccgagttcaaagaggcgtttagcctgtttgacaaggacggagatggcacgatcacgacaaaggagctcggtacggtaatgcgatctctcggccagaaccccactgaggctgaactgcaggacatgatcaacgaggtcgacgccgacggctccggaacgatagatttccctgagttcctcacaatgatggcaagaaagatgaaggacaccgactcggaggaggagatccgagaggcgttccgcgtattcgacaaggatggcaacggtttcatttcggcggccgagctcaggcacgttatgaccaaccttggcgagaagcttacggacgaggaggtagatgagatgattcgggaggcagatattgacggtgatggtcaggtcaactacgaggagttcgtcaccatgatgacgtccaagtaaatatatgataatgttggctgtctcgtgtaatgtcgtgagaaagaaagcgcgcgcgaaagaaagagagaaaggaatagaaaactataaatagcttgttgttaaagcaacgcaacaacaagctgtaagcaacaaacaatatttacgaagtatacgataatgaaagtcgacagggaagcaagcacggatatatatgaaaactaagcgaaatgacgtcgtcatcatcaccagcagcagcagcagcagcagcagcagcagcagcagcaccaccaccaccaccatcacgaccaccaccaccaccaccaccatcacgaccaccac

SEQ ID NO: 70 - RNA, 1136 nt, Varroa destructor
  source 1..1136: mol_type = mRNA
gttcggctcggggactagctgatcggtcggtgtgtattgttggctattggcaaagaccgttgttgagtgggctcgctgcactgagcgtttaaatctggtgaaatcgttggcaatggcggatcagctgaccgaggagcaaatcgccgaattcaaggaggctttcagcctgttcgataaagacggtgatggcacaattacgaccaaggaactagggaccgtcatgcggtccctcggccagaaccctactgaggctgagcttcaagacatgatcaacgaggtcgacgctgacggtaacggcactattgactttccagagtttctcacgatgatggcgcgtaaaatgaaggacaccgactccgaggaggagatccgggaagcttttagggtttttgataaagacggaaatggcttcatttcggctgcagagctgaggcacgtaatgaccaaccttggcgaaaagctcacggacgaggaagtggacgagatgatccgcgaggcggatatcgacggcgacggacaggtcaactacgaggagttcgtcacgatgatgacatcaaaatgaagggctcactattgcgcgggaaaagcagcccaacaaagaaacctagagtgcgaaagcgagaacgttaaacacgatgatatgctaatgataatacatacgactagaggacagaaagacagacagacagactgagcagacgaacgggcaagttgaagaaaagccgagttgaactggctaaccgttgggtactcattcatattcgatagttacagacaacaacatgaaaaacgacagcaacaatccgcaacaaacacacggagattgcacacaatgagggtaaactgaacatggtgcagcggaagttggatggcagcggtacacagtgctgctactgctgctgctgcaaatgctaacactcaataatgataataataattataattatagaaatatgattatgttgtccaaaagagaaacaaacaacacaaacaaagctattaaaaatctgaataaaagctaagaagaaatcaagtagcagtcgacatggggagtggcaaacgataagagtccacagaaaactgaagcggccgaaagaaaacacgaggcaaaaggcaatgtattcattaagacgagg

SEQ ID NO: 71 - RNA, 150 nt, synthetic construct
  misc_feature 1..150: note = Synthetic Sequence
  source 1..150: mol_type = other RNA
gtttagcccattttcgcgctgtgtctgtcggcgactgcggaagtactgagctagctgagtcgtggtgcttagaaggcagtggcagtgagtggcatcagcggtaataagtgagggacaacagccggagagtcgtgtcggttggtaggtcgg

SEQ ID NO: 72 - RNA, 150 nt, synthetic construct
  misc_feature 1..150: note = Synthetic Sequence
  source 1..150: mol_type = other RNA
ttcgttgccgattaatgccccacatgtgagttacgtgcggtgcttgttccgctgtgcattggacgttacatacagagagtcaggtgtagtttattttagacgaaaaactaccagcactacctgatacagcgacccaacgtagagagggaa

SEQ ID NO: 73 - RNA, 150 nt, synthetic construct
  misc_feature 1..150: note = synthetic sequence
  source 1..150: mol_type = other RNA
agagaaagagctgttgtttcgctaggttagttctgaacaattggattgattcgcaaatgtacgcttgtacggctaacggttgaacggactgtgcaaactgcggatagtgcgtgaactagcagacaacatatccaataactaactacacgg

SEQ ID NO: 74 - RNA, 150 nt, synthetic construct
  misc_feature 1..150: note = synthetic sequence
  source 1..150: mol_type = other RNA
ttattattgatacaaacacctagttcagacagacaatttcggtttgctttgttggcactataaggagtaagtgatattgttttttgtttatcagtcggcctagtattgtcggtgcacttacggataacaggagaggcagtacaaaggcaa

SEQ ID NO: 75 - RNA, 150 nt, synthetic construct
  misc_feature 1..150: note = synthetic sequence
  source 1..150: mol_type = other RNA
ctcgattcactatcgttcacgggcgctaatacagcgaaatattaataggcaaagcaagcaagccagccggccaagcccaaaccccgggcgaaacgcagataccaacagcgctgaagtgcgtggcaaacgacaacgggacagtagggcata

SEQ ID NO: 76 - RNA, 150 nt, synthetic construct
  misc_feature 1..150: note = synthetic sequence
  source 1..150: mol_type = other RNA
agctagacagcatatagcttttctaaccatggctgatcagctaactgaggaacagatcgccgagttcaaagaggcgtttagcctgtttgacaaggacggagatggcacgatcacgacaaaggagctcggtacggtaatgcgatctctcgg

SEQ ID NO: 77 - RNA, 150 nt, synthetic construct
  misc_feature 1..150: note = synthetic sequence
  source 1..150: mol_type = other RNA
ccagaaccccactgaggctgaactgcaggacatgatcaacgaggtcgacgccgacggctccggaacgatagatttccctgagttcctcacaatgatggcaagaaagatgaaggacaccgactcggaggaggagatccgagaggcgttccg

SEQ ID NO: 78 - RNA, 150 nt, synthetic construct
  misc_feature 1..150: note = synthetic sequence
  source 1..150: mol_type = other RNA
cgtattcgacaaggatggcaacggtttcatttcggcggccgagctcaggcacgttatgaccaaccttggcgagaagcttacggacgaggaggtagatgagatgattcgggaggcagatattgacggtgatggtcaggtcaactacgagga

SEQ ID NO: 79 - RNA, 150 nt, synthetic construct
  misc_feature 1..150: note = synthetic sequence
  source 1..150: mol_type = other RNA
gttcgtcaccatgatgacgtccaagtaaatatatgataatgttggctgtctcgtgtaatgtcgtgagaaagaaagcgcgcgcgaaagaaagagagaaaggaatagaaaactataaatagcttgttgttaaagcaacgcaacaacaagctg

SEQ ID NO: 80 - RNA, 150 nt, synthetic construct
  misc_feature 1..150: note = synthetic sequence
  source 1..150: mol_type = other RNA
taagcaacaaacaatatttacgaagtatacgataatgaaagtcgacagggaagcaagcacggatatatatgaaaactaagcgaaatgacgtcgtcatcatcaccagcagcagcagcagcagcagcagcagcagcagcagcaccaccacca

SEQ ID NO: 81 - RNA, 150 nt, synthetic construct
  misc_feature 1..150: note = synthetic sequence
  source 1..150: mol_type = other RNA
gttcggctcggggactagctgatcggtcggtgtgtattgttggctattggcaaagaccgttgttgagtgggctcgctgcactgagcgtttaaatctggtgaaatcgttggcaatggcggatcagctgaccgaggagcaaatcgccgaatt

SEQ ID NO: 82 - RNA, 150 nt, synthetic construct
  misc_feature 1..150: note = synthetic sequence
  source 1..150: mol_type = other RNA
caaggaggctttcagcctgttcgataaagacggtgatggcacaattacgaccaaggaactagggaccgtcatgcggtccctcggccagaaccctactgaggctgagcttcaagacatgatcaacgaggtcgacgctgacggtaacggcac

SEQ ID NO: 83 - RNA, 150 nt, synthetic construct
  misc_feature 1..150: note = synthetic sequence
  source 1..150: mol_type = other RNA
tattgactttccagagtttctcacgatgatggcgcgtaaaatgaaggacaccgactccgaggaggagatccgggaagcttttagggtttttgataaagacggaaatggcttcatttcggctgcagagctgaggcacgtaatgaccaacct

SEQ ID NO: 84 - RNA, 150 nt, synthetic construct
  misc_feature 1..150: note = synthetic sequence
  source 1..150: mol_type = other RNA
tggcgaaaagctcacggacgaggaagtggacgagatgatccgcgaggcggatatcgacggcgacggacaggtcaactacgaggagttcgtcacgatgatgacatcaaaatgaagggctcactattgcgcgggaaaagcagcccaacaaag

SEQ ID NO: 85 - RNA, 150 nt, synthetic construct
  misc_feature 1..150: note = synthetic sequence
  source 1..150: mol_type = other RNA
aaacctagagtgcgaaagcgagaacgttaaacacgatgatatgctaatgataatacatacgactagaggacagaaagacagacagacagactgagcagacgaacgggcaagttgaagaaaagccgagttgaactggctaaccgttgggta

SEQ ID NO: 86 - RNA, 150 nt, synthetic construct
  misc_feature 1..150: note = synthetic sequence
  source 1..150: mol_type = other RNA
ctcattcatattcgatagttacagacaacaacatgaaaaacgacagcaacaatccgcaacaaacacacggagattgcacacaatgagggtaaactgaacatggtgcagcggaagttggatggcagcggtacacagtgctgctactgctgc

SEQ ID NO: 87 - RNA, 150 nt, synthetic construct
  misc_feature 1..150: note = synthetic sequence
  source 1..150: mol_type = other RNA
tgctgcaaatgctaacactcaataatgataataataattataattatagaaatatgattatgttgtccaaaagagaaacaaacaacacaaacaaagctattaaaaatctgaataaaagctaagaagaaatcaagtagcagtcgacatggg

SEQ ID NO: 88 - RNA, 156 nt, synthetic construct
  misc_feature 1..156: note = synthetic sequence
  source 1..156: mol_type = other RNA
ggcaacggtttcatttcggcggccgagctcaggcacgttatgaccaaccttggcgagaagcttacggacgaggaggtagatgagatgattcgggaggcagatattgacggtgatggtcaggtcaactacgaggagttcgtcaccatgatgacgtcc

SEQ ID NO: 89 - RNA, 257 nt, synthetic construct
  misc_feature 1..257: note = synthetic sequence
  source 1..257: mol_type = other RNA
ggcggatcagctgaccgaggagcaaatcgccgaattcaaggaggctttcagcctgttcgataaagacggtgatggcacaattacgaccaaggaactagggaccgtcatgcggtccctcggccagaaccctactgaggctgagcttcaagacatgatcaacgaggtcgacgctgacggtaacggcactattgactttccagagtttctcacgatgatggcgcgtaaaatgaaggacaccgactccgaggaggagatcc

SEQ ID NO: 90 - RNA, 420 nt, synthetic construct
  misc_feature 1..420: note = synthetic sequence
  source 1..420: mol_type = other RNA
ggaacagatcgccgagttcaaagaggcgtttagcctgtttgacaaggacggagatggcacgatcacgacaaaggagctcggtacggtaatgcgatctctcggccagaacccactgaggctgaactgcaggacatgatcaacgaggtcgacgccgacggctccggaacgatagatttccctgagttcctcacaatgatgcaagaaagatgaaggacaccgactcggaggaggagatcgagaggcgttccgcgtattcgacaaggatgcaacggtttcatttcggcggccgagctcaggcacgttatgaccaaccttggcgagaagcttacggacgaggaggtagatgagatgattcgggaggcagatattgacggtgatggtcaggtcaactacgaggagttcgtcaccatgatgacgtcc

SEQ ID NO: 91 - RNA, 372 nt, synthetic construct
  misc_feature 1..372: note = synthetic sequence
  source 1..372: mol_type = other RNA
ggaacagatcgccgagttcaaagaggcgtttagcctgtttgacaaggacggagatggcacgatcacgacaaaggagctcggtacggtaatgcgatctctcggccagaacccactgaggctgaactgcaggacatgatcaacgaggtcgacgccgacggctccggaacgatagatttccctgagttcctcacaatgatgcaagaaagatgaaggacaccgactcggaggaggagatcgagaggcgttccgcgtattcgacaaggatgcaacggtttcatttcggcggccgagctcaggcacgttatgaccaaccttggcgagaagcttacggacgaggaggtagatgagatgattcgggaggcagatattgac

SEQ ID NO: 92 - RNA, 477 nt, synthetic construct
  misc_feature 1..477: note = synthetic sequence
  source 1..477: mol_type = other RNA
ggcggatcagctgaccgaggagcaaatcgccgaattcaaggaggctttcagcctgttcgataaagacggtgatggcacaattacgaccaaggaactagggaccgtcatgcggtccctcggccagaaccctactgaggctgagcttcaagacatgatcaacgaggtcgacgctgacggtaacggcactattgactttccagagtttctcacgatgatggcgcgtaaaatgaaggacaccgactccgaggagggatccgggaagcttttagggtttttgataaagacggaaatggcttcatttcggctgcagagctgaggcacgtaatgaccaaccttggcgaaaagctcacggacgaggaagtggacgagatgatccgcgaggcggatatcgacggcgacggacaggtcaactacgaggagttcgtcacgatgatgacatcaaaatgaagggctcactattgcgcgggaaaagcagcc

SEQ ID NO: 93 - DNA, 373 bp, synthetic construct
  misc_feature 1..373: note = synthetic sequence
  source 1..373: mol_type = other DNA
ggagatcgccgagttcaaagaggcgtttagcctgtttgacaaggacggagatggcacgatcacgacaaaggagctcggtacggtaatgcgatctctcggccagaaccccactgaggctgaactgcaggacatgatcaacgaggtcgacgccgacggctccggaacgatagatttccctgagttcctcacaatgatggcaagaaagatgaaggacaccgactcggaggaggagatccgagaggcgttccgcgtattcgacaaggatggcaacggtttcatttcggcggccgagctcaggcacgttatgaccaaccttggcgagaagcttacggacgaggaggtagatgagatgattcgggaggcagatattgac

SEQ ID NO: 94 - RNA, 376 nt, synthetic construct
  misc_feature 1..376: note = synthetic sequence
  source 1..376: mol_type = other RNA
ggaacagatcgccgagttcaaagaggcgtttagcctgtttgacaaggacggagatggcacgatcacgacaaaggagctcggtacggtaatgcgatctctcggccagaaccccactgaggctgaactgcaggacatgatcaacgaggtcgacgccgacggctccggaacgatagatttccctgagttcctcacaatgatggcaagaaagatgaaggacaccgactcggaggaggagatccgagaggcgttccgcgtattcgacaaggatggcaacggtttcatttcggcggccgagctcaggcacgttatgaccaaccttggcgagaagcttacggacgaggaggtagatgagatgattcgggaggcagatattgac